SEQUENCE LISTING (12 sequences)

SEQ ID NO: 1 - DNA, 17719 bp, synthetic construct
  misc_feature 1..17719: note = synthesized DNA of PHP48070
  source 1..17719: mol_type = other DNA
cgcgccggaattaattaggtaatttcacgcgccggatccttaattaagtctagagtcgactgtttaattctagtggccggcccagctgatgatcccggtgaagttcctattccgaagttcctattctccagaaagtataggaacttcactagagcttgcggccgctcgagttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgtcgaggcctcacgtgttaacaagcttgcatgccggtttaaacagtcgaggtcgacggtatcgataagcttgttaacagaagttcctattccgaagttcctattctctagaaagtataggaacttccaccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagtgggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgactagctagtcagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcccccaagcttatcgataccgtcggcgcggggtacgttagctgattaagtcagcatgcgcggccggcgtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaggtacccatcacttaagtggcgcgccgtcgacggatccgtacgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcgcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttgagggaaaagggttgaggaacttatgcatcttatatctctccacctccaggattttaagccctagttactcaacccttttccctcagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataagcggcgcaagggcgaattctgcagatatccatcacacttgggccgcttctagctagctagggtttgggtagtgagtgtaataaagttgcaaagtttttggttaggttacgttttgaccttattattatagttcaaagggaaacattaattaaaggggattatgaaggtggatgatgcctgattggattgaggatcttactgggtgaattgagctgcttagctatggatcccacagttctacccatcaataagtgcttttgtggtagtcttgtggcttccatatctggggagcttcatttgcctttatagtattaaccttcttccaatccagcatcatccaccacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgatttttggattgttggatttttataaacaaatctggggcccaagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacgtacgtctttccacaatacataactattaattaatcttaaataaataaaggataaaatatttttttttcttcataaagttaaaatatgttattttttgtttagatgtatattcgaataaatctaaatatatgataatgattttttatattgattaaacatataatcaatattaaatatgatatttttttatataggttgtacacataattttataaggataaaaaatatgataaaaataaattttaaatatttttatatttacgagaaaaaaaaatattttagccataaataaatgaccagcatattttacaaccttagtaattcataaattcctatatgtatatttgaaattaaaaacagataatcgttaagggaaggaatcctacgtcatctcttgccatttgtttttcatgcaaacagaaagggacgaaaaaccacctcaccatgaatcactcttcacaccatttttactagcaaacaagtctcaacaactgaagccagctctctttccgtttctttttacaacactttctttgaaatagtagtattttttttcacatgatttattaacgtgccaaaagatgcttattgaatagagtgcacatttgtaatgtactactaattagaacatgaaaaagcattgttctaacacgataatcctgtgaaggcgttaactccaaagatccaatttcactatataaattgtgacgaaagcaaaatgaattcacatagctgagagagaaaggaaaggttaactaagaagcaatacttcagcggccgcttctagctagctagggtttgggtagtgagtgtaataaagttgcaaagtttttggttaggttacgttttgaccttattattatagttcaaagggaaacattaattaaaggggattatgaagtgggctctcttgattcttggatgaggatcttactgggtgaattgagctgcttagctatggatcccacagttctacccatcaataagtgcttttgtggtagtcttgtggcttccatatctggggagcttcatttgcctttatagtattaaccttctccaagaacaaagagagcccacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggccgcatgagccgtaaaggttcaatacaacgagtgcttgttttcttagggacaagcattgtacttatgtatgattctgtgtaaccatgagtcttccacgttgtactaatgtgaagggcaaaaataaaacacagaacaagttcgtttttctcaaataatgtgaaggtagaaaatggaaccatgcctcctctcttgcatgtgatttaaaatattagcagatggtacgtcgagtcgacctgcaggtcgactcgacgtacgtcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccgctagtcgactaagtcatcaactattccaagctacgtatttgggagtttgtggagtacagcaagatgatatacctagacggtgatatccaagtttttgacaacattgaccacttgtttgacttgcctgataactacttctatgcggtgatggactgtttctgtgagccaacttggggccacactaaacaatatcagatcggttactgccagcagtgcccccataaggttcagtggcccactcactttgggcccaaacctcctctctatttcaatgctggcatgtttgtgtatgagcccaatttggctacttaccgtgacctccttcaaacagtccaagtcacccagcccacttcctttgctgaacaggattttttgaacatgtacttcaaggacaaatataggccaattcctaatgtctacaatcttgtgctggccatgctgtggcgtcaccctgagaacgttgagcttgacaaagttaaagtggttcactactgtgctgctgggtctaagccttggaggtacactgggaagtgactcgaggtcatcaattactccaagctacgtatttgggagttcgtggagtacaagaagacgatatacctagacggtgacatccaagtatttggaaacatagaccacttgtttgatctgcctgataattatttctatgcggtgatggattgtttctgcgagaagacttggagccacacccctcagttccagattgggtactgccaacagtgccctgataaggttcaatggccctctcactttggttccaaacctcctctatatttcaatgctggcatgtttgtttatgagcctaatctcgacacctaccgtgatcttctccaaactgtccaactcaccaagcccacttcttttgctgagcaggactttctcaacatgtacttcaaggacaagtacaagccaataccgaacatgtacaaccttgtgctggccatgttgtggcgtcaccctgaaaatgttgaacttgataaagttcaagtggttcattactgtgctgctgggtctaagccttggaggttcactgggaagtaactgcaggtcatcaactactccaagctccgtatatgggagtttgtggagtacagcaagatgatatacttggacggagacattgaggtatatgagaacatagaccacctatttgacctacctgatggtaacttttacgctgtgatggattgtttctgcgagaagacatggagtcacacccctcagtacaaggtgggttactgccagcaatgcccggagaaggtgcggtggcccaccgaattgggtcagcccccttctctttacttcaacgctggcatgttcgtgttcgaacccaacatcgccacctatcatgacctattgaaaacggtgcaagtcaccactcccacctcgttcgctgaacaagatttcttgaacatgtacttcaaggacatttacaagccaatccctttaaattacaatcttgtcctcgccatgctgtggcgccacccggaaaacgttaaattagaccaagtcaaggttgttcactattgcgcagcggggtccaagccatggagatatacggggaagtagcctaggcgtacgcaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtcctaggctacttccccgtatatctccatggcttggaccccgctgcgcaatagtgaacaaccttgacttggtctaatttaacgttttccgggtggcgccacagcatggcgaggacaagattgtaatttaaagggattggcttgtaaatgtccttgaagtacatgttcaagaaatcttgttcagcgaacgaggtgggagtggtgacttgcaccgttttcaataggtcatgataggtggcgatgttgggttcgaacacgaacatgccagcgttgaagtaaagagaagggggctgacccaattcggtgggccaccgcaccttctccgggcattgctggcagtaacccaccttgtactgaggggtgtgactccatgtcttctcgcagaaacaatccatcacagcgtaaaagttaccatcaggtaggtcaaataggtggtctatgttctcatatacctcaatgtctccgtccaagtatatcatcttgctgtactccacaaactcccatatacggagcttggagtagttgatgacctgcagttacttcccagtgaacctccaaggcttagacccagcagcacagtaatgaaccacttgaactttatcaagttcaacattttcagggtgacgccacaacatggccagcacaaggttgtacatgttcggtattggcttgtacttgtccttgaagtacatgttgagaaagtcctgctcagcaaaagaagtgggcttggtgagttggacagtttggagaagatcacggtaggtgtcgagattaggctcataaacaaacatgccagcattgaaatatagaggaggtttggaaccaaagtgagagggccattgaaccttatcagggcactgttggcagtacccaatctggaactgaggggtgtggctccaagtcttctcgcagaaacaatccatcaccgcatagaaataattatcaggcagatcaaacaagtggtctatgtttccaaatacttggatgtcaccgtctaggtatatcgtcttcttgtactccacgaactcccaaatacgtagcttggagtaattgatgacctcgagtcacttcccagtgtacctccaaggcttagacccagcagcacagtagtgaaccactttaactttgtcaagctcaacgttctcagggtgacgccacagcatggccagcacaagattgtagacattaggaattggcctatatttgtccttgaagtacatgttcaaaaaatcctgttcagcaaaggaagtgggctgggtgacttggactgtttgaaggaggtcacggtaagtagccaaattgggctcatacacaaacatgccagcattgaaatagagaggaggtttgggcccaaagtgagtgggccactgaaccttatgggggcactgctggcagtaaccgatctgatattgtttagtgtggccccaagttggctcacagaaacagtccatcaccgcatagaagtagttatcaggcaagtcaaacaagtggtcaatgttgtcaaaaacttggatatcaccgtctaggtatatcatcttgctgtactccacaaactcccaaatacgtagcttggaatagttgatgacttagtcgactagcggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccggtctaggccaaggccgcgaagttaaaagcaatgttgtcacttgtacgtactaacacatgatgtgatagtttatgctagctagctataacataagctgtctctgagtgtgttgtatattaataaagatcatcactggtgaatggtgatcgtgtacgtaccctacttagtaggcaatggaagcacttagagtgtgctttgtgcatggccttgcctctgttttgagacttttgtaatgttttcgagtttaaatctttgcctttgcgtacgtgggcggatcccctgcaggagatccaagcttgg

SEQ ID NO: 2 - DNA, 17679 bp, synthetic construct
  misc_feature 1..17679: note = synthesized DNA of PHP50573
  source 1..17679: mol_type = other DNA
ggagatccaagcttggcgcgccggaattaattaggtaatttcacgcgccggatccttaattaagtctagagtcgactgtttaattctagtggccggcccagctgatgatcccggtgaagttcctattccgaagttcctattctccagaaagtataggaacttcactagagcttgcggccgctcgagttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgtcgaggcctcacgtgttaacaagcttgcatgccggtttaaacagtcgaggtcgacggtatcgataagcttgttaacagaagttcctattccgaagttcctattctctagaaagtataggaacttccaccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagtgggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgactagctagtcagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcccccaagcttatcgataccgtcggcgcggggtacgttagctgattaagtcagcatgcgcggccggcgtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaggtacccatcacttaagtggcgcgccgtcgacggatccgtacgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcatgactatcgactcacaatactacaagtcgcgagacaaaaacgacacggcacccaaaatcgcgggaatccgatatgccccgctatcgacaccattactcaaccgatgtgagaccttctctctggtctggcacattttcagcattcccactttcctcacaattttcatgctatgctgcgcaattccactgctctggccatttgtgattgcgtatgtagtgtacgctgttaaagacgactccccgtccaacggaggagtggtcaagcgatactcgcctatttcaagaaacttcttcatctggaagctctttggccgctacttccccataactctgcacaagacggtggatctggagcccacgcacacatactaccctctggacgtccaggagtatcacctgattgctgagagatactggccgcagaacaagtacctccgagcaatcatctccaccatcgagtactttctgcccgccttcatgaaacggtctctttctatcaacgagcaggagcagcctgccgagcgagatcctctcctgtctcccgtttctcccagctctccgggttctcaacctgacaagtggattaaccacgacagcagatatagccgtggagaatcatctggctccaacggccacgcctcgggctccgaacttaacggcaacggcaacaatggcaccactaaccgacgacctttgtcgtccgcctctgctggctccactgcatctgattccacgcttcttaacgggtccctcaactcctacgccaaccagatcattggcgaaaacgacccacagctgtcgcccacaaaactcaagcccactggcagaaaatacatcttcggctaccacccccacggcattatcggcatgggagcctttggtggaattgccaccgagggagctggatggtccaagctctttccgggcatccctgtttctcttatgactctcaccaacaacttccgagtgcctctctacagagagtacctcatgagtctgggagtcgcttctgtctccaagaagtcctgcaaggccctcctcaagcgaaaccagtctatctgcattgtcgttggtggagcacaggaaagtcttctggccagacccggtgtcatggacctggtgctactcaagcgaaagggttttgttcgacttggtatggaggtcggaaatgtcgcccttgttcccatcatggcctttggtgagaacgacctctatgaccaggttagcaacgacaagtcgtccaagctgtaccgattccagcagtttgtcaagaacttccttggattcacccttcctttgatgcatgcccgaggcgtcttcaactacgatgtcggtcttgtcccctacaggcgacccgtcaacattgtggttggttcccccattgacttgccttatctcccacaccccaccgacgaagaagtgtccgaataccacgaccgatacatcgccgagctgcagcgaatctacaacgagcacaaggatgaatatttcatcgattggaccgaggagggcaaaggagccccagagttccgaatgattgagtaagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacgtacgtctttccacaatacataactattaattaatcttaaataaataaaggataaaatatttttttttcttcataaagttaaaatatgttattttttgtttagatgtatattcgaataaatctaaatatatgataatgattttttatattgattaaacatataatcaatattaaatatgatatttttttatataggttgtacacataattttataaggataaaaaatatgataaaaataaattttaaatatttttatatttacgagaaaaaaaaatattttagccataaataaatgaccagcatattttacaaccttagtaattcataaattcctatatgtatatttgaaattaaaaacagataatcgttaagggaaggaatcctacgtcatctcttgccatttgtttttcatgcaaacagaaagggacgaaaaaccacctcaccatgaatcactcttcacaccatttttactagcaaacaagtctcaacaactgaagccagctctctttccgtttctttttacaacactttctttgaaatagtagtattttttttcacatgatttattaacgtgccaaaagatgcttattgaatagagtgcacatttgtaatgtactactaattagaacatgaaaaagcattgttctaacacgataatcctgtgaaggcgttaactccaaagatccaatttcactatataaattgtgacgaaagcaaaatgaattcacatagctgagagagaaaggaaaggttaactaagaagcaatacttcagcggccgcttctagctagctagggtttgggtagtgagtgtaataaagttgcaaagtttttggttaggttacgttttgaccttattattatagttcaaagggaaacattaattaaaggggattatgaagtgggctctcttgattcttggatgaggatcttactgggtgaattgagctgcttagctatggatcccacagttctacccatcaataagtgcttttgtggtagtcttgtggcttccatatctggggagcttcatttgcctttatagtattaaccttctccaagaacaaagagagcccacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggccgcatgagccgtaaaggttcaatacaacgagtgcttgttttcttagggacaagcattgtacttatgtatgattctgtgtaaccatgagtcttccacgttgtactaatgtgaagggcaaaaataaaacacagaacaagttcgtttttctcaaataatgtgaaggtagaaaatggaaccatgcctcctctcttgcatgtgatttaaaatattagcagatggtacgtcgagtcgacctgcaggtcgactcgacgtacgtcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccgctagtcgactaagtcatcaactattccaagctacgtatttgggagtttgtggagtacagcaagatgatatacctagacggtgatatccaagtttttgacaacattgaccacttgtttgacttgcctgataactacttctatgcggtgatggactgtttctgtgagccaacttggggccacactaaacaatatcagatcggttactgccagcagtgcccccataaggttcagtggcccactcactttgggcccaaacctcctctctatttcaatgctggcatgtttgtgtatgagcccaatttggctacttaccgtgacctccttcaaacagtccaagtcacccagcccacttcctttgctgaacaggattttttgaacatgtacttcaaggacaaatataggccaattcctaatgtctacaatcttgtgctggccatgctgtggcgtcaccctgagaacgttgagcttgacaaagttaaagtggttcactactgtgctgctgggtctaagccttggaggtacactgggaagtgactcgaggtcatcaattactccaagctacgtatttgggagttcgtggagtacaagaagacgatatacctagacggtgacatccaagtatttggaaacatagaccacttgtttgatctgcctgataattatttctatgcggtgatggattgtttctgcgagaagacttggagccacacccctcagttccagattgggtactgccaacagtgccctgataaggttcaatggccctctcactttggttccaaacctcctctatatttcaatgctggcatgtttgtttatgagcctaatctcgacacctaccgtgatcttctccaaactgtccaactcaccaagcccacttcttttgctgagcaggactttctcaacatgtacttcaaggacaagtacaagccaataccgaacatgtacaaccttgtgctggccatgttgtggcgtcaccctgaaaatgttgaacttgataaagttcaagtggttcattactgtgctgctgggtctaagccttggaggttcactgggaagtaactgcaggtcatcaactactccaagctccgtatatgggagtttgtggagtacagcaagatgatatacttggacggagacattgaggtatatgagaacatagaccacctatttgacctacctgatggtaacttttacgctgtgatggattgtttctgcgagaagacatggagtcacacccctcagtacaaggtgggttactgccagcaatgcccggagaaggtgcggtggcccaccgaattgggtcagcccccttctctttacttcaacgctggcatgttcgtgttcgaacccaacatcgccacctatcatgacctattgaaaacggtgcaagtcaccactcccacctcgttcgctgaacaagatttcttgaacatgtacttcaaggacatttacaagccaatccctttaaattacaatcttgtcctcgccatgctgtggcgccacccggaaaacgttaaattagaccaagtcaaggttgttcactattgcgcagcggggtccaagccatggagatatacggggaagtagcctaggcgtacgcaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtcctaggctacttccccgtatatctccatggcttggaccccgctgcgcaatagtgaacaaccttgacttggtctaatttaacgttttccgggtggcgccacagcatggcgaggacaagattgtaatttaaagggattggcttgtaaatgtccttgaagtacatgttcaagaaatcttgttcagcgaacgaggtgggagtggtgacttgcaccgttttcaataggtcatgataggtggcgatgttgggttcgaacacgaacatgccagcgttgaagtaaagagaagggggctgacccaattcggtgggccaccgcaccttctccgggcattgctggcagtaacccaccttgtactgaggggtgtgactccatgtcttctcgcagaaacaatccatcacagcgtaaaagttaccatcaggtaggtcaaataggtggtctatgttctcatatacctcaatgtctccgtccaagtatatcatcttgctgtactccacaaactcccatatacggagcttggagtagttgatgacctgcagttacttcccagtgaacctccaaggcttagacccagcagcacagtaatgaaccacttgaactttatcaagttcaacattttcagggtgacgccacaacatggccagcacaaggttgtacatgttcggtattggcttgtacttgtccttgaagtacatgttgagaaagtcctgctcagcaaaagaagtgggcttggtgagttggacagtttggagaagatcacggtaggtgtcgagattaggctcataaacaaacatgccagcattgaaatatagaggaggtttggaaccaaagtgagagggccattgaaccttatcagggcactgttggcagtacccaatctggaactgaggggtgtggctccaagtcttctcgcagaaacaatccatcaccgcatagaaataattatcaggcagatcaaacaagtggtctatgtttccaaatacttggatgtcaccgtctaggtatatcgtcttcttgtactccacgaactcccaaatacgtagcttggagtaattgatgacctcgagtcacttcccagtgtacctccaaggcttagacccagcagcacagtagtgaaccactttaactttgtcaagctcaacgttctcagggtgacgccacagcatggccagcacaagattgtagacattaggaattggcctatatttgtccttgaagtacatgttcaaaaaatcctgttcagcaaaggaagtgggctgggtgacttggactgtttgaaggaggtcacggtaagtagccaaattgggctcatacacaaacatgccagcattgaaatagagaggaggtttgggcccaaagtgagtgggccactgaaccttatgggggcactgctggcagtaaccgatctgatattgtttagtgtggccccaagttggctcacagaaacagtccatcaccgcatagaagtagttatcaggcaagtcaaacaagtggtcaatgttgtcaaaaacttggatatcaccgtctaggtatatcatcttgctgtactccacaaactcccaaatacgtagcttggaatagttgatgacttagtcgactagcggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccggtctaggccaaggccgcgaagttaaaagcaatgttgtcacttgtacgtactaacacatgatgtgatagtttatgctagctagctataacataagctgtctctgagtgtgttgtatattaataaagatcatcactggtgaatggtgatcgtgtacgtaccctacttagtaggcaatggaagcacttagagtgtgctttgtgcatggccttgcctctgttttgagacttttgtaatgttttcgagtttaaatctttgcctttgcgtacgtgggcggatcccctgca

SEQ ID NO: 3 - DNA, 22374 bp, synthetic construct
  misc_feature 1..22374: note = synthesized DNA of PHP64612
  source 1..22374: mol_type = other DNA
cctcacgtgttaacagaagttcctattccgaagttcctattctctagaaagtataggaacttccaccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcagccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagtgggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgactagctagtcagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcccccaagcttatcgataccgtcggcgcggggtacgttagctgattaagtcagcatgcgcggccggcgtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaggtacccatcacttaagtggcgcgccgtcgacggatccgtacccatctgcaggtaaattgcagctgaaggacagtgaagggtgaatttatccatttaaaccattttctttttaacacatttcttatggtaatctcttctcactacactataaaaatggcttctcaatcccattttctacatcatcccattctattgagttttgtttatttgctttcacttttttttttatctgcctcttcccttaatttgcttgacttcttcttcacattttgctttgttttctcctccggcttccggtatttcaaattcaagatgagcaagttgaaatttataaatagaaatacagatattatttacaacgtcaaatctttggtattttcaatatttgaatggggtaaatttgtcatatagtcatcatcactgactacttatctaacctatttaatttggagcatattctttataaggtccctctcacggccaatgtctaattattgatatacagctcttgttttctagtgctgcttataatattatctacacatatatatggtactgcacactactactatatagtagtaagtaaactagcaacagccggggccaaactccaataactaggcattggggtttagttggtaatataaatataacatcaaaaagtctttgcttgtgacgaacatcacaatgcacccaccattgatgccacgacagacattgttaattttttttttaatttttaaaaaagaagcaattccaatagttctatattacaatctcacgtgatccaagcacaacgtttcattttttgtacatgctcgatatataaataatatttcattttatagtaaaatataatgacattttcgaatataatttttgaaatttcattttccaaatgaaatactaatattaatattaatgagattaccacaaatcatgttatgaatgaaataaagagttttggcattctaactttctttgaatagaacaaaatgtatacaacactctccatatatacacgatttattcagggatcatatacattctctcatgattaacatagtctgctttcttcacgtctaagcagataatttttggtccacaagataaaattatcattagtcgttttaattaattccttgagcatcaagcactaaaataattaaacttctccattaccaaaaaaaaaagataggtgattcagtaacatgtagtactagtactactgattttttttttcttttgattttaatgaatggttcgtatcgagcatcgagaaatccatttattaggtgtgtaatgtaatagtagtatttccttgattttcagtaataagatggattcttacatttatatctgtttgacagaaaatgttgtcaatgcatttcttgggcacaaagttttttgaaacatgaattaattttttcaaaatatttatgacatcaaattgaccctaaaataagtgataaagctttaacgtggaatgacattaatttttccatgataaataaaacacttaaaacattttaatattaatattataatcagttacaactatgttcaattaatgcaataacttttaaataaatattaaaatattttttttctgttctccaataaagagatcttgttgcacggaaaaagtcacattcttatttagtaaaaaattataattattgtttgaaaaatatcattttcactgcagaaaatttgatccagctctacagatcatacttttattgtacaataatacaataaaaatattcatctgcaggaaatatcattttcattgtacaataatataaagataaatatataccagaaaagaaaaagaaactgatgtggcacaatgtattcactgaaagaatgcatattgtatttcacctttcaagcagcactaagaatatacttcttttattatacttgtgcatttactcaaccaccctcggtggagtaagaaagaagatagataaaagttttttttgacatttggtgaatctcttaattaaaaaaataaaataatccatttcctttatttaatttcttttttcccatctgtgaaattccaattctgcttcgcgctcctgtctataaattgacttagccaccacctcagtttccattcattcacttcttctctttataccccccctctcttttttgcgttcattctgttttcgtaagtactgttgtttttctcttctatttctttttttgtttgtgttgtttttttttcttccttatcgttgttctgcctctcctctgtttcggtgctctgttcaccacttccacgtgagaatgatcttccttctttgcatgttcattctctcgtgaccactggatcagactccatgttctgatccagggtctctctctaacgcctgtactttcatccatgaccaccttaaaaacaacatgggggtggtgctgttacactaactctgtttctggggtgctgtctttgttcaattttactcagaaaatatcttttcttggattctattcggtgtgtgggaacatgatcctgtcggtcggttgtttttaggttaatccttaactggttacaaggatctaacgcttgaatgcatgtcctgagttaaagaaacaaaagaagaacacacctagtacagcctggcctcgaaccaagaacttctttgttggtttctcattattactaaaataaaataaagtatacgttttcttttttctttgggatgaacggttcagacttatgagaagtttaagctaatcctgtagtggagtgttcaatttattttaaactttaaagcaatagctcaagcactaaacttctttttcaagttcaaccactttggtagcttgctaattgctgctattgttctaattaattaatgtaattattgtttaaaaaagaaaagttggtgacactggaataaaaaagtgtactatctggcaattattcttctgcagcaatgtttgaggttgaaatcttagtagaacaaagtagaagatctggtatttatattttttgtagacagatggtgggggtgggtggtaggccttgaaatccaatatagttttgtagaataattttattattttttttttttgctcacttgtttgtggtattgattttgtgatgactcaagattaatgatttaccttcatttttttcatggtgacatattatgtatattcttgatctgtttcttacacttctttttcgttgttgtagctgttgaagtctgcggccgcatgaagaggtctccagcatcttcttgttcatcatctacttcctctgttgggtttgaagctcccattgaaaaaagaaggcctaagcatccaaggaggaataatttgaagtcacaaaaatgcaagcagaaccaaaccaccactggtggcagaagaagctctatctatagaggagttacaaggcataggtggacagggaggtttgaagctcacctatgggataagagctcttggaacaacattcagagcaagaagggtcgacaagtttatttgggggcatatgatactgaagaatctgcagcccgtacctatgaccttgcagcccttaaatactggggaaaagatgcaaccctgaatttcccgatagaaacttataccaaggagctcgaggaaatggacaaggtttcaagagaagaatatttggcttctttgcggcgccaaagcagtggcttttctagaggcctgtctaagtaccgtggggttgctaggcatcatcataatggtcgctgggaagcacgaattggaagagtatgcggaaacaagtacctctacttggggacatataaaactcaagaggaggcagcagtggcatatgacatggcagcaatagagtaccgtggagtcaatgcagtgaccaattttgacataagcaactacatggacaaaataaagaagaaaaatgaccaaacccaacaacaacaaacagaagcacaaacggaaacagttcctaactcctctgactctgaagaagtagaagtagaacaacagacaacaacaataaccacaccacccccatctgaaaatctgcacatgccaccacagcagcaccaagttcaatacaccccccatgtctctccaagggaagaagaatcatcatcactgatcacaattatggaccatgtgcttgagcaggatctgccatggagcttcatgtacactggcttgtctcagtttcaagatccaaacttggctttctgcaaaggtgatgatgacttggtgggcatgtttgatagtgcagggtttgaggaagacattgattttctgttcagcactcaacctggtgatgagactgagagtgatgtcaacaatatgagcgcagttttggatagtgttgagtgtggagacacaaatggggctggtggaagcatgatgcatgtggataacaagcagaagatagtatcatttgcttcttcaccatcatctacaactacagtttcttgtgactatgctctagatctatgagcggccgcatttcgcaccaaatcaatgaaagtaataatgaaaagtctgaataagaatacttaggcttagatgcctttgttacttgtgtaaaataacttgagtcatgtacctttggcggaaacagaataaataaaaggtgaaattccaatgctctatgtataagttagtaatacttaatgtgttctacggttgtttcaatatcatcaaactctaattgaaactttagaaccacaaatctcaatcttttcttaatgaaatgaaaaatcttaattgtaccatgtttatgttaaacaccttacaattaattggttggagaggaggaccaaccgatgggacaacattgggagaaagagattcaatggagatttggataggagaacaacattctttttcacttcaatacaagatgagtgcaacactaaggatatgtatgagactttcagaagctacgacaacatagatgagtgaggtggtgattcctagcaagaaagacattagaggaagccaaaatcgaacaaggaagacatcaagggcaagagacaggaccatccatctcaggaaaaggagctttgggatagtccgagaagttgtacaagaaattttttggagggtgagtgatgcattgctggtgactttaactcaatcaaaattgagaaagaaagaaaagggagggggctcacatgtgaatagaagggaaacgggagaattttacagttttgatctaatgggcatcccagctagtggtaacatattcaccatgtttaaccttcacgtacgaaaccaactgcgtttggggctccagattaaacgacgccgtttcgttcctttcgcttcacggcttaacgatgtcgtttctgtctgtgcccaaaaaataaaggcatttgttatttgcaccagatatttactaagtgcaccctagtttgacaagtaggcgataattacaaatagatgcggtgcaaataataaattttgaaggaaataattacaaaagaacagaacttatatttactttattttaaaaaactaaaatgaaagaacaaaaaaagtaaaaaatacaaaaaatgtgctttaaccactttcattatttgttacagaaagtatgattctactcaaattgatctgttgtatctggtgctgccttgtcacactggcgatttcaatcccctaaagatatggtgcaaactgcgaagtgatcaatatctgctcggttaatttagattaattaataatattcaacgtgatgtaccaaaaaaagacaattttttgctccattgacaaattaaacctcatcaaggtaatttccaaacctataagcaaaaaaatttcacattaattggcccgcaatcctattagtcttattatactagagtaggaaaaaaaacaattacacaacttgtcttattattctctatgctaatgaatatttttcccttttgttagaaatcagtgtttcctaatttattgagtattaattccactcaccgcatatatttaccgttgaataagaaaattttacacataattctttttaagataaataatttttttatactagatcttatatgattacgtgaagccaagtgggttatactaatgatatataatgtttgatagtaatcagtttataaaccaaatgcatggaaatgttacgtggaagcacgtaaattaacaagcattgaagcaaatgcagccaccgcaccaaaaccaccccacttcacttccacgtaccatattccatgcaactacaacaccctaaaacttcaataaatgcccccaccttcacttcacttcacccatcaatagcaagcggccgcaccatggcgatttccgatgagcctgaaagtgtagccactgctctcaaccactcttccctgcgccgccgtccctccgccacctccaccgccggcctcttcaattcgcctgagacaaccaccgacagttccggtgatgacttggccaaggattctggttccgacgactccatcaacaacgacgacgccgccgtcaattcccaacagcaaaacgaaaaacaagacactgatttctccgtcctcaaattcgcctaccgtccttccgtccccgctcaccgcaaagtgaaggaaagtccgctcagctccgacactattttccgtcagagtcacgcgggcctcttcaacctttgtatagtagtccttgttgctgtgaatagccgactcatcattgagaatttaatgaagtatggttggttgatcaaatctggcttttggtttagtgcaaagtcattgagagactggccccttttcatgtgttgtctttctcttgtggtatttcctttcgctgcctttatggtggagaagttggcacaacggaagtgtatacccgaaccagttgttgttgtacttcatataatcattacctcaacttcgcttttctatccagttttagttattctcaagtgtgattctgcttttgtatcaggtgtcacgttaatgctgttttcttgtgttgtatggttaaaattggtgtcttttgcacatacaaactatgatatgagagcacttaccaaattagttgaaaagggagaagcactgctcgatactctgaacatggagtatccttacaacgtaaccttcaagagcttggcatatttcctgcttgcccctacattatgttaccagccaagctatcctcgcacaccttatattcgaaagggttggttgtttcgccaacttgtcaagctgatagtatttacaggagttatgggatttataatagaacaatatattaatcccatagtacaaaattcacagcatcctctcaagggaaaccttctttacgccaccgagagagttctgaagctttctgttccaaatttatatgtgtggctctgcatgttctattgctttttccacctttggttaaatatcgtggcagagcttcttcgatttggtgatcgtgaattctacaaggattggtggaatgccaaaactgtcgaagattattggaggatgtggaatatgcctgttcacaaatggatgatccgccacctatattttccatgtttaaggcacggtctaccaaaggctgctgctcttttaatttccttcctggtttctgctttattccatgagctgtgcattgctgttccttgccacatgttcaagttgtgggctttcggtggaattatgtttcaggttcctttggtcttgatcactaattatctgcaaaataaattcaaaaactcaatggttggaaatatgattttttggttcatattcagtatcgttggtcaacctatgtgtgtactgctatactaccatgacttgatgaataggaaaggcaaacttgactgagcggccgcgaagttaaaagcaatgttgtcacttgtcgtactaacacatgatgtgatagtttatgctagctagctataacataagctgtctctgagtgtgttgtatattaataaagatcatcactggtgaatggtgatcgtgtacgtaccctacttagtaggcaatggaagcacttagagtgtgctttgtgcatggccttgcctctgttttgagacttttgtaatgttttcgagtttaaatctttgcctttgcgtacgtctttccacaatacataactattaattaatcttaaataaataaaggataaaatatttttttttcttcataaagttaaaatatgttattttttgtttagatgtatattcgaataaatctaaatatatgataatgattttttatattgattaaacatataatcaatattaaatatgatatttttttatataggttgtacacataattttataaggataaaaaatatgataaaaataaattttaaatatttttatatttacgagaaaaaaaaatattttagccataaataaatgaccagcatattttacaaccttagtaattcataaattcctatatgtatatttgaaattaaaaacagataatcgttaagggaaggaatcctacgtcatctcttgccatttgtttttcatgcaaacagaaagggacgaaaaaccacctcaccatgaatcactcttcacaccatttttactagcaaacaagtctcaacaactgaagccagctctctttccgtttctttttacaacactttctttgaaatagtagtattttttttcacatgatttattaacgtgccaaaagatgcttattgaatagagtgcacatttgtaatgtactactaattagaacatgaaaaagcattgttctaacacgataatcctgtgaaggcgttaactccaaagatccaatttcactatataaattgtgacgaaagcaaaatgaattcacatagctgagagagaaaggaaaggttaactaagaagcaatacttcagcggccgcatgccgaatcccgaggctcaccacccctcccggtcccgggcccggccctccacgtcagccgcggcccgccccccggcccgggcccgcgtctccctccgccagcttctgcgcgtggcatcagtcgcgagcggcatccagttcgggtgggccttacagctctctctgctgacgccctacgttcagcagctggggatcccccaccaatgggccagcatcatctggctctgcggcccagtctccggcctcttcgtgcagcccctcgtcggccacatgagcgaccgctgcaccagccgctacggccgccgcaggcccttcatcctcgtcggcgccgtcgccatcgtcgccgctgttctcgtcatcgcttacgccgccgacatcggctggctcctcggcgacaccgcggactaccgccctgccgccatcaccgtcttcatcgtcggcttctggatcctcgacgtcgctaacaacgtcacgcaaggtccctgccgtgccttgctcggtgatctcactagcaaggatcctcgaaggacacgtgttgcaaatgcttattactcactgtttatggccattggtaacattcttggctatgcaactggatcatatagtggttggtacaagatttttacttttgccctttcccctgcttgcacaattagttgtgcaaatctcaagtctgctttctttcttgacattgctttcattgcggtcacaacatatatcagcatcatggcagctcatgaagtgcctctaaattcaagtgaggcggcccatgctgaagcaggggcaggggagtcaggtagtgcagaagaagctttcatgtgggaattatttgggacattcaaatattttacaacccctgtatggataattctgtctgttactgctctgacatggattgggtggttcccatttactctctttgatactgattggatgggtcgagagatttatggtggtgatccaaatcaaggccttgtttatgatactggagttagaatgggagcacttggtttgttgcttaattcagttgttcttgcattaacatcattgttcatggagaggctatgcaggaagaggggagctggttttgtgtggggaatctcaaatatcatgatgaccgtttgctttcttgcaatgctagtagtaacctatgtggcaaataacatgggctatataggcaaagatttaccaccaactggcattgtgatagctgcgttgattatctttaccattcttgggtttccactggcaatcacttatagtgttccatatgccttaatttccacacatattgagtcattgggactcggccaagggttatcaatgggtgtcctaaatctggcaatagtggtcccacagataatagtgtcactgggaagtggaccatgggatcagctatttggtggaggaaactccccagcctttgctgtggcagctgtttcagcccttatcagtggactcatagctgtgttggctattcctcgatctggtgctcaaaaggctcgaagccatgtatgagcggccgcctgaacgggaattaaacctataaacataaatataaataatatatataaacctaagtgtctaagttccataaattaagctgtagtctctggcttaaaacatgttaggtttgtttatacaagtagttggatgtttggagtacttcggtcttttgcgtaccatcaatatttaagaactaagttagttatgttccgtaacttatgggctcttaattaaactatatctgcacaaaattatatatatatcaaatgtgatggtatgtggactataaaaagatatggttgagaaccacaaactttgaaacttcgaataatatattgccagtgacagtcttgttgatttgttatagcaagtcctattttcttaatcattgctttgttttaacgtacctagatttcataacttttgtctttgtctcaagctgaacctaatgatgatagtaatattaacttattgtataggggtatttcataggataaaaaatgatgtgcaattacgtgtagaccaaatattacttgatgacagatggcctgcaggatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgctagtcgactaagtcatcaactattccaagctacgtatttgggagtttgtggagtacagcaagatgatatacctagacggtgatatccaagtttttgacaacattgaccacttgtttgacttgcctgataactacttctatgcggtgatggactgtttctgtgagccaacttggggccacactaaacaatatcagatcggttactgccagcagtgcccccataaggttcagtggcccactcactttgggcccaaacctcctctctatttcaatgctggcatgtttgtgtatgagcccaatttggctacttaccgtgacctccttcaaacagtccaagtcacccagcccacttcctttgctgaacaggattttttgaacatgtacttcaaggacaaatataggccaattcctaatgtctacaatcttgtgctggccatgctgtggcgtcaccctgagaacgttgagcttgacaaagttaaagtggttcactactgtgctgctgggtctaagccttggaggtacactgggaagtgactcgaggtcatcaattactccaagctacgtatttgggagttcgtggagtacaagaagacgatatacctagacggtgacatccaagtatttggaaacatagaccacttgtttgatctgcctgataattatttctatgcggtgatggattgtttctgcgagaagacttggagccacacccctcagttccagattgggtactgccaacagtgccctgataaggttcaatggccctctcactttggttccaaacctcctctatatttcaatgctggcatgtttgtttatgagcctaatctcgacacctaccgtgatcttctccaaactgtccaactcaccaagcccacttcttttgctgagcaggactttctcaacatgtacttcaaggacaagtacaagccaataccgaacatgtacaaccttgtgctggccatgttgtggcgtcaccctgaaaatgttgaacttgataaagttcaagtggttcattactgtgctgctgggtctaagccttggaggttcactgggaagtaactgcaggtcatcaactactccaagctccgtatatgggagtttgtggagtacagcaagatgatatacttggacggagacattgaggtatatgagaacatagaccacctatttgacctacctgatggtaacttttacgctgtgatggattgtttctgcgagaagacatggagtcacacccctcagtacaaggtgggttactgccagcaatgcccggagaaggtgcggtggcccaccgaattgggtcagcccccttctctttacttcaacgctggcatgttcgtgttcgaacccaacatcgccacctatcatgacctattgaaaacggtgcaagtcaccactcccacctcgttcgctgaacaagatttcttgaacatgtacttcaaggacatttacaagccaatccctttaaattacaatcttgtcctcgccatgctgtggcgccacccggaaaacgttaaattagaccaagtcaaggttgttcactattgcgcagcggggtccaagccatggagatatacggggaagtagcctaggcgtacgcaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtcctaggctacttccccgtatatctccatggcttggaccccgctgcgcaatagtgaacaaccttgacttggtctaatttaacgttttccgggtggcgccacagcatggcgaggacaagattgtaatttaaagggattggcttgtaaatgtccttgaagtacatgttcaagaaatcttgttcagcgaacgaggtgggagtggtgacttgcaccgttttcaataggtcatgataggtggcgatgttgggttcgaacacgaacatgccagcgttgaagtaaagagaagggggctgacccaattcggtgggccaccgcaccttctccgggcattgctggcagtaacccaccttgtactgaggggtgtgactccatgtcttctcgcagaaacaatccatcacagcgtaaaagttaccatcaggtaggtcaaataggtggtctatgttctcatatacctcaatgtctccgtccaagtatatcatcttgctgtactccacaaactcccatatacggagcttggagtagttgatgacctgcagttacttcccagtgaacctccaaggcttagacccagcagcacagtaatgaaccacttgaactttatcaagttcaacattttcagggtgacgccacaacatggccagcacaaggttgtacatgttcggtattggcttgtacttgtccttgaagtacatgttgagaaagtcctgctcagcaaaagaagtgggcttggtgagttggacagtttggagaagatcacggtaggtgtcgagattaggctcataaacaaacatgccagcattgaaatatagaggaggtttggaaccaaagtgagagggccattgaaccttatcagggcactgttggcagtacccaatctggaactgaggggtgtggctccaagtcttctcgcagaaacaatccatcaccgcatagaaataattatcaggcagatcaaacaagtggtctatgtttccaaatacttggatgtcaccgtctaggtatatcgtcttcttgtactccacgaactcccaaatacgtagcttggagtaattgatgacctcgagtcacttcccagtgtacctccaaggcttagacccagcagcacagtagtgaaccactttaactttgtcaagctcaacgttctcagggtgacgccacagcatggccagcacaagattgtagacattaggaattggcctatatttgtccttgaagtacatgttcaaaaaatcctgttcagcaaaggaagtgggctgggtgacttggactgtttgaaggaggtcacggtaagtagccaaattgggctcatacacaaacatgccagcattgaaatagagaggaggtttgggcccaaagtgagtgggccactgaaccttatgggggcactgctggcagtaaccgatctgatattgtttagtgtggccccaagttggctcacagaaacagtccatcaccgcatagaagtagttatcaggcaagtcaaacaagtggtcaatgttgtcaaaaacttggatatcaccgtctaggtatatcatcttgctgtactccacaaactcccaaatacgtagcttggaatagttgatgacttagtcgactagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacctgcagggcatgcaagcttggcgcgccggaattaattaggtaatttcacgcgccggatccttaattaagtctagagtcgactgtttaattctagtggccggcctctgcctgcgttctgctgtggaagttcctattccgaagttcctattctccagaaagtataggaacttcacatgctgcctcgtgcaagtcacgatctcgagttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgtcgagg

SEQ ID NO: 4 - DNA, 22047 bp, synthetic construct
  misc_feature 1..22047: note = synthesized DNA of PHP64613
  source 1..22047: mol_type = other DNA
cctcacgtgttaacagaagttcctattccgaagttcctattctctagaaagtataggaacttccaccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcagccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagtgggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgactagctagtcagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcccccaagcttatcgataccgtcggcgcggggtacgttagctgattaagtcagcatgcgcggccggcgtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaggtacccatcacttaagtggcgcgccgtcgacggatccgtacccatctgcaggtaaattgcagctgaaggacagtgaagggtgaatttatccatttaaaccattttctttttaacacatttcttatggtaatctcttctcactacactataaaaatggcttctcaatcccattttctacatcatcccattctattgagttttgtttatttgctttcacttttttttttatctgcctcttcccttaatttgcttgacttcttcttcacattttgctttgttttctcctccggcttccggtatttcaaattcaagatgagcaagttgaaatttataaatagaaatacagatattatttacaacgtcaaatctttggtattttcaatatttgaatggggtaaatttgtcatatagtcatcatcactgactacttatctaacctatttaatttggagcatattctttataaggtccctctcacggccaatgtctaattattgatatacagctcttgttttctagtgctgcttataatattatctacacatatatatggtactgcacactactactatatagtagtaagtaaactagcaacagccggggccaaactccaataactaggcattggggtttagttggtaatataaatataacatcaaaaagtctttgcttgtgacgaacatcacaatgcacccaccattgatgccacgacagacattgttaattttttttttaatttttaaaaaagaagcaattccaatagttctatattacaatctcacgtgatccaagcacaacgtttcattttttgtacatgctcgatatataaataatatttcattttatagtaaaatataatgacattttcgaatataatttttgaaatttcattttccaaatgaaatactaatattaatattaatgagattaccacaaatcatgttatgaatgaaataaagagttttggcattctaactttctttgaatagaacaaaatgtatacaacactctccatatatacacgatttattcagggatcatatacattctctcatgattaacatagtctgctttcttcacgtctaagcagataatttttggtccacaagataaaattatcattagtcgttttaattaattccttgagcatcaagcactaaaataattaaacttctccattaccaaaaaaaaaagataggtgattcagtaacatgtagtactagtactactgattttttttttcttttgattttaatgaatggttcgtatcgagcatcgagaaatccatttattaggtgtgtaatgtaatagtagtatttccttgattttcagtaataagatggattcttacatttatatctgtttgacagaaaatgttgtcaatgcatttcttgggcacaaagttttttgaaacatgaattaattttttcaaaatatttatgacatcaaattgaccctaaaataagtgataaagctttaacgtggaatgacattaatttttccatgataaataaaacacttaaaacattttaatattaatattataatcagttacaactatgttcaattaatgcaataacttttaaataaatattaaaatattttttttctgttctccaataaagagatcttgttgcacggaaaaagtcacattcttatttagtaaaaaattataattattgtttgaaaaatatcattttcactgcagaaaatttgatccagctctacagatcatacttttattgtacaataatacaataaaaatattcatctgcaggaaatatcattttcattgtacaataatataaagataaatatataccagaaaagaaaaagaaactgatgtggcacaatgtattcactgaaagaatgcatattgtatttcacctttcaagcagcactaagaatatacttcttttattatacttgtgcatttactcaaccaccctcggtggagtaagaaagaagatagataaaagttttttttgacatttggtgaatctcttaattaaaaaaataaaataatccatttcctttatttaatttcttttttcccatctgtgaaattccaattctgcttcgcgctcctgtctataaattgacttagccaccacctcagtttccattcattcacttcttctctttataccccccctctcttttttgcgttcattctgttttcgtaagtactgttgtttttctcttctatttctttttttgtttgtgttgtttttttttcttccttatcgttgttctgcctctcctctgtttcggtgctctgttcaccacttccacgtgagaatgatcttccttctttgcatgttcattctctcgtgaccactggatcagactccatgttctgatccagggtctctctctaacgcctgtactttcatccatgaccaccttaaaaacaacatgggggtggtgctgttacactaactctgtttctggggtgctgtctttgttcaattttactcagaaaatatcttttcttggattctattcggtgtgtgggaacatgatcctgtcggtcggttgtttttaggttaatccttaactggttacaaggatctaacgcttgaatgcatgtcctgagttaaagaaacaaaagaagaacacacctagtacagcctggcctcgaaccaagaacttctttgttggtttctcattattactaaaataaaataaagtatacgttttcttttttctttgggatgaacggttcagacttatgagaagtttaagctaatcctgtagtggagtgttcaatttattttaaactttaaagcaatagctcaagcactaaacttctttttcaagttcaaccactttggtagcttgctaattgctgctattgttctaattaattaatgtaattattgtttaaaaaagaaaagttggtgacactggaataaaaaagtgtactatctggcaattattcttctgcagcaatgtttgaggttgaaatcttagtagaacaaagtagaagatctggtatttatattttttgtagacagatggtgggggtgggtggtaggccttgaaatccaatatagttttgtagaataattttattattttttttttttgctcacttgtttgtggtattgattttgtgatgactcaagattaatgatttaccttcatttttttcatggtgacatattatgtatattcttgatctgtttcttacacttctttttcgttgttgtagctgttgaagtctgcggccgcatgaagaggtctccagcatcttcttgttcatcatctacttcctctgttgggtttgaagctcccattgaaaaaagaaggcctaagcatccaaggaggaataatttgaagtcacaaaaatgcaagcagaaccaaaccaccactggtggcagaagaagctctatctatagaggagttacaaggcataggtggacagggaggtttgaagctcacctatgggataagagctcttggaacaacattcagagcaagaagggtcgacaagtttatttgggggcatatgatactgaagaatctgcagcccgtacctatgaccttgcagcccttaaatactggggaaaagatgcaaccctgaatttcccgatagaaacttataccaaggagctcgaggaaatggacaaggtttcaagagaagaatatttggcttctttgcggcgccaaagcagtggcttttctagaggcctgtctaagtaccgtggggttgctaggcatcatcataatggtcgctgggaagcacgaattggaagagtatgcggaaacaagtacctctacttggggacatataaaactcaagaggaggcagcagtggcatatgacatggcagcaatagagtaccgtggagtcaatgcagtgaccaattttgacataagcaactacatggacaaaataaagaagaaaaatgaccaaacccaacaacaacaaacagaagcacaaacggaaacagttcctaactcctctgactctgaagaagtagaagtagaacaacagacaacaacaataaccacaccacccccatctgaaaatctgcacatgccaccacagcagcaccaagttcaatacaccccccatgtctctccaagggaagaagaatcatcatcactgatcacaattatggaccatgtgcttgagcaggatctgccatggagcttcatgtacactggcttgtctcagtttcaagatccaaacttggctttctgcaaaggtgatgatgacttggtgggcatgtttgatagtgcagggtttgaggaagacattgattttctgttcagcactcaacctggtgatgagactgagagtgatgtcaacaatatgagcgcagttttggatagtgttgagtgtggagacacaaatggggctggtggaagcatgatgcatgtggataacaagcagaagatagtatcatttgcttcttcaccatcatctacaactacagtttcttgtgactatgctctagatctatgagcggccgcatttcgcaccaaatcaatgaaagtaataatgaaaagtctgaataagaatacttaggcttagatgcctttgttacttgtgtaaaataacttgagtcatgtacctttggcggaaacagaataaataaaaggtgaaattccaatgctctatgtataagttagtaatacttaatgtgttctacggttgtttcaatatcatcaaactctaattgaaactttagaaccacaaatctcaatcttttcttaatgaaatgaaaaatcttaattgtaccatgtttatgttaaacaccttacaattaattggttggagaggaggaccaaccgatgggacaacattgggagaaagagattcaatggagatttggataggagaacaacattctttttcacttcaatacaagatgagtgcaacactaaggatatgtatgagactttcagaagctacgacaacatagatgagtgaggtggtgattcctagcaagaaagacattagaggaagccaaaatcgaacaaggaagacatcaagggcaagagacaggaccatccatctcaggaaaaggagctttgggatagtccgagaagttgtacaagaaattttttggagggtgagtgatgcattgctggtgactttaactcaatcaaaattgagaaagaaagaaaagggagggggctcacatgtgaatagaagggaaacgggagaattttacagttttgatctaatgggcatcccagctagtggtaacatattcaccatgtttaaccttcacgtacgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcatgactatcgactcacaatactacaagtcgcgagacaaaaacgacacggcacccaaaatcgcgggaatccgatatgccccgctatcgacaccattactcaaccgatgtgagaccttctctctggtctggcacattttcagcattcccactttcctcacaattttcatgctatgctgcgcaattccactgctctggccatttgtgattgcgtatgtagtgtacgctgttaaagacgactccccgtccaacggaggagtggtcaagcgatactcgcctatttcaagaaacttcttcatctggaagctctttggccgctacttccccataactctgcacaagacggtggatctggagcccacgcacacatactaccctctggacgtccaggagtatcacctgattgctgagagatactggccgcagaacaagtacctccgagcaatcatcaccaccatcgagtactttctgcccgccttcatgaaacggtctctttctatcaacgagcaggagcagcctgccgagcgagatcctctcctgtctcccgtttctcccagctctccgggttctcaacctgacaagtggattaaccacgacagcagatatagccgtggagaatcatctggctccaacggccacgcctcgggctccgaacttaacggcaacggcaacaacggcaccactaaccgacgacctttgtcgtccgcctctgctggctccactgcatctgattccacgcttcttaacgggtccctcaactcctacgccaaccagatcattggcgaaaacgacccacagctgtcgcccacaaaactcaagcccactggcagaaaatacatcttcggctaccacccccacggcattatcggcatgggagcctttggtggaattgccaccgagggagctggatggtccaagctctttccgggcatccctgtttctcttatgactctcaccaacaacttccgagtgcctctctacagagagtacctcatgagtctgggagtcgcttctgtctccaagaagtcctgcaaggccctcctcaagcgaaaccagtctatctgcattgtcgttggtggagcacaggaaagtcttctggccagacccggtgtcatggacctggtgctactcaagcgaaagggttttgttcgacttggtatggaggtcggaaatgtcgcccttgttcccatcatggcctttggtgagaacgacctctatgaccaggttagcaacgacaagtcgtccaagctgtaccgattccagcagtttgtcaagaacttccttggattcacccttcctttgatgcatgcccgaggcgtcttcaactacgatgtcggtcttgtcccctacaggcgacccgtcaacattgtggttggttcccccattgacttgccttatctcccacaccccaccgacgaagaagtgtccgaataccacgaccgatacatcgccgagctgcagcgaatctacaacgagcacaaggatgaatatttcatcgattggaccgaggagggcaaaggagccccagagttccgaatgattgagtaagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacgtactttctagacgtacgtctttccacaatacataactattaattaatcttaaataaataaaggataaaatatttttttttcttcataaagttaaaatatgttattttttgtttagatgtatattcgaataaatctaaatatatgataatgattttttatattgattaaacatataatcaatattaaatatgatatttttttatataggttgtacacataattttataaggataaaaaatatgataaaaataaattttaaatatttttatatttacgagaaaaaaaaatattttagccataaataaatgaccagcatattttacaaccttagtaattcataaattcctatatgtatatttgaaattaaaaacagataatcgttaagggaaggaatcctacgtcatctcttgccatttgtttttcatgcaaacagaaagggacgaaaaaccacctcaccatgaatcactcttcacaccatttttactagcaaacaagtctcaacaactgaagccagctctctttccgtttctttttacaacactttctttgaaatagtagtattttttttcacatgatttattaacgtgccaaaagatgcttattgaatagagtgcacatttgtaatgtactactaattagaacatgaaaaagcattgttctaacacgataatcctgtgaaggcgttaactccaaagatccaatttcactatataaattgtgacgaaagcaaaatgaattcacatagctgagagagaaaggaaaggttaactaagaagcaatacttcagcggccgcatgccgaatcccgaggctcaccacccctcccggtcccgggcccggccctccacgtcagccgcggcccgccccccggcccgggcccgcgtctccctccgccagcttctgcgcgtggcatcagtcgcgagcggcatccagttcgggtgggccttacagctctctctgctgacgccctacgttcagcagctggggatcccccaccaatgggccagcatcatctggctctgcggcccagtctccggcctcttcgtgcagcccctcgtcggccacatgagcgaccgctgcaccagccgctacggccgccgcaggcccttcatcctcgtcggcgccgtcgccatcgtcgccgctgttctcgtcatcgcttacgccgccgacatcggctggctcctcggcgacaccgcggactaccgccctgccgccatcaccgtcttcatcgtcggcttctggatcctcgacgtcgctaacaacgtcacgcaaggtccctgccgtgccttgctcggtgatctcactagcaaggatcctcgaaggacacgtgttgcaaatgcttattactcactgtttatggccattggtaacattcttggctatgcaactggatcatatagtggttggtacaagatttttacttttgccctttcccctgcttgcacaattagttgtgcaaatctcaagtctgctttctttcttgacattgctttcattgcggtcacaacatatatcagcatcatggcagctcatgaagtgcctctaaattcaagtgaggcggcccatgctgaagcaggggcaggggagtcaggtagtgcagaagaagctttcatgtgggaattatttgggacattcaaatattttacaacccctgtatggataattctgtctgttactgctctgacatggattgggtggttcccatttactctctttgatactgattggatgggtcgagagatttatggtggtgatccaaatcaaggccttgtttatgatactggagttagaatgggagcacttggtttgttgcttaattcagttgttcttgcattaacatcattgttcatggagaggctatgcaggaagaggggagctggttttgtgtggggaatctcaaatatcatgatgaccgtttgctttcttgcaatgctagtagtaacctatgtggcaaataacatgggctatataggcaaagatttaccaccaactggcattgtgatagctgcgttgattatctttaccattcttgggtttccactggcaatcacttatagtgttccatatgccttaatttccacacatattgagtcattgggactcggccaagggttatcaatgggtgtcctaaatctggcaatagtggtcccacagataatagtgtcactgggaagtggaccatgggatcagctatttggtggaggaaactccccagcctttgctgtggcagctgtttcagcccttatcagtggactcatagctgtgttggctattcctcgatctggtgctcaaaaggctcgaagccatgtatgagcggccgcctgaacgggaattaaacctataaacataaatataaataatatatataaacctaagtgtctaagttccataaattaagctgtagtctctggcttaaaacatgttaggtttgtttatacaagtagttggatgtttggagtacttcggtcttttgcgtaccatcaatatttaagaactaagttagttatgttccgtaacttatgggctcttaattaaactatatctgcacaaaattatatatatatcaaatgtgatggtatgtggactataaaaagatatggttgagaaccacaaactttgaaacttcgaataatatattgccagtgacagtcttgttgatttgttatagcaagtcctattttcttaatcattgctttgttttaacgtacctagatttcataacttttgtctttgtctcaagctgaacctaatgatgatagtaatattaacttattgtataggggtatttcataggataaaaaatgatgtgcaattacgtgtagaccaaatattacttgatgacagatggcctgcaggtcgactcgacgtacgtcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccgctctagaccacctaacatcaccaccgttgttgccaatgtcaccaccgagcaattacccaaggctcgtggaggaagtgggcgtgccttcgtgacctttcttgctggaaacggtgattatgtaaagggtgttgtgggtttggccaaaggactgagaaaggccaaaagcatgtaccctttggtggttgctgtgttaccagatgttcctgaagaacatcgtgcgattctcaaatcccaaggttgcattgtcagggagattgaacctgtgtaccctcctgagaaccagacccagttcgggatccatattacgtcatcaactattccaagctacgtatttgggagtttgtggagttcagcaagatgatatacctagacggtgatatacaagtgtttgacaatattgaccacttgtttgacttgcctgataactacttttatgcggtgatggactgtttttgtgagcccacttggggccacactctgcagtcatgacctattgaaaacggtgcaagtcaccactcccacctcgttcgctgaacaagatttcttgaacatgtacttcaaggacatttacaggccaatccctttaaattacaatcttgtcctcgccatgctgtggcgccacccggaaaacgttaaattagaccaagtcaaggttgttcactattgcgcagcggggtccaagccatggagatatacggggaaggaagagaatatgcagagggaggacataaagatgctggtgaagaaatggtgggatatctacaatgatgcttcgcttgactacaagccattgatgaatgcaagtgaagctccagcagcggatggtgttgacattgaacaattcgtgcaggctctatcagaggttggtcatgttcaatatgtcaccgaattctatgtcctccctctgcatattctcttccttccccgtatatctccatggcttggaccccgctgcgcaatagtgaacaaccttgacttggtctaatttaacgttttccgggtggcgccacagcatggcgaggacaagattgtaatttaaagggattggcttgtaaatgtccttgaagtacatgttcaagaaatcttgttcagcgaacgaggtgggagtggtgacttgcaccgttttcaataggtcatgaaagcttacacaaacatgccagcattgaaatagagaggaggcttgggcccaaagtgagtgggccactgaaccttatgagggcactgctggcagtatccgatttgatactgcagagtgtggccccaagtgggctcacaaaaacagtccatcaccgcataaaagtagttatcaggcaagtcaaacaagtggtcaatattgtcaaacacttgtatatcaccgtctaggtatatcatcttgctgaactccacaaactcccaaatacgtagcttggaatagttgatgacgtaatatgtcgaccgaactgggtctggttctcaggagggtacacaggttcaatctccctgacaatgcaaccttgggatttgagaatctcacgatgttcttcaggaacatctggtaacacagcaaccaccaaagggtacatgcttttggcctttctcagtcctttggccaaacccacgacaccctttacgtaatcaccgttcccagcaagaaaggtcacgaaggcacgcccacttcctccacgagccttgggtaattgctcggtggtgacattggcaacaacggtggtgatgttaggtgggcggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccggtctaggccaaggccgcgaagttaaaagcaatgttgtcacttgtacgtactaacacatgatgtgatagtttatgctagctagctataacataagctgtctctgagtgtgttgtatattaataaagatcatcactggtgaatggtgatcgtgtacgtaccctacttagtaggcaatggaagcacttagagtgtgctttgtgcatggccttgcctctgttttgagacttttgtaatgttttcgagtttaaatctttgcctttgcgtacgtgggcggatcccctgcagggcatgcaagcttggcgcgccggaattaattaggtaatttcacgcgccggatccttaattaagtctagagtcgactgtttaattctagtggccggcctctgcctgcgttctgctgtggaagttcctattccgaagttcctattctccagaaagtataggaacttcacatgctgcctcgtgcaagtcacgatctcgagttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgtcgagg

SEQ ID NO: 5 - DNA, 4376 bp, synthetic construct
  misc_feature 1..4376: note = synthesized DNA of PHP25066A
  source 1..4376: mol_type = other DNA
cgcgccaagcttggatctcctgcaggatctggccggccggatctcgtacggatccgtcgactctagaggccggccgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcgtcatcaactattccaagctacgtatttgggagtttgtggagtacagcaagatgatatacctagacggtgatatccaagtttttgacaacattgaccacttgtttgacttgcctgataactacttctatgcggtgatggactgtttctgtgagccaacttggggccacactaaacaatatcagatcggttactgccagcagtgcccccataaggttcagtggcccactcactttgggcccaaacctcctctctatttcaatgctggcatgtttgtgtatgagcccaatttggctacttaccgtgacctccttcaaacagtccaagtcacccagcccacttcctttgctgaacaggattttttgaacatgtacttcaaggacaaatataggccaattcctaatgtctacaatcttgtgctggccatgctgtggcgtcaccctgagaacgttgagcttgacaaagttaaagtggttcactactgtgctgctgggtctaagccttggaggtacactgggaagtgactcgaggtcatcaattactccaagctacgtatttgggagttcgtggagtacaagaagacgatatacctagacggtgacatccaagtatttggaaacatagaccacttgtttgatctgcctgataattatttctatgcggtgatggattgtttctgcgagaagacttggagccacacccctcagttccagattgggtactgccaacagtgccctgataaggttcaatggccctctcactttggttccaaacctcctctatatttcaatgctggcatgtttgtttatgagcctaatctcgacacctaccgtgatcttctccaaactgtccaactcaccaagcccacttcttttgctgagcaggactttctcaacatgtacttcaaggacaagtacaagccaataccgaacatgtacaaccttgtgctggccatgttgtggcgtcaccctgaaaatgttgaacttgataaagttcaagtggttcattactgtgctgctgggtctaagccttggaggttcactgggaagtaactgcaggtcatcaactactccaagctccgtatatgggagtttgtggagtacagcaagatgatatacttggacggagacattgaggtatatgagaacatagaccacctatttgacctacctgatggtaacttttacgctgtgatggattgtttctgcgagaagacatggagtcacacccctcagtacaaggtgggttactgccagcaatgcccggagaaggtgcggtggcccaccgaattgggtcagcccccttctctttacttcaacgctggcatgttcgtgttcgaacccaacatcgccacctatcatgacctattgaaaacggtgcaagtcaccactcccacctcgttcgctgaacaagatttcttgaacatgtacttcaaggacatttacaagccaatccctttaaattacaatcttgtcctcgccatgctgtggcgccacccggaaaacgttaaattagaccaagtcaaggttgttcactattgcgcagcggggtccaagccatggagatatacggggaagtagcggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaatgtgttattaacccttctcttcgaggacctgcaggtcgacgg

SEQ ID NO: 6 - DNA, 15128 bp, synthetic construct
  misc_feature 1..15128: note = synthesized DNA of PHP64207A
  source 1..15128: mol_type = other DNA
cgcgccggtaccgggccccccctcgagtggcgttagctgattaagtcagcatgcgcggccggccgcaagctctagtgaagttcctatactttctggagaataggaacttcggaataggaacttcaccgggatcatcagctgggccggccggtacctcagctgatgggtctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgaatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctcccactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcggctgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggtggaagttcctatactttctagagaataggaacttcggaataggaacttctgttgttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtcaaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttgggaagggcgaattccagcacactggcggccgttactagtgtttgatcctctagacgtacgaaaccaactgcgtttggggctccagattaaacgacgccgtttcgttcctttcgcttcacggcttaacgatgtcgtttctgtctgtgcccaaaaaataaaggcatttgttatttgcaccagatatttactaagtgcaccctaatttgacaagtaggcgataattacaaatagatgcggtgcaaataataaattttgaaggaaataattacaaaagaacagaacttatatttactttattttaaaaaactaaaatgaaagaacaaaaaaagtaaaaaatacaaaaaatgtgctttaaccactttcattatttgttacagaaagtatgattctactcaaattgatctgttgtatctggtgctgccttgtcacactggcgatttcaatcccctaaagatatggtgcaaactgcgaagtgatcaatatctgctcggttaatttagattaattaataatattcaacgtgatgtaccaaaaaaagacaattttttgctccattgtcaaattaaacctcatcaaggtaatttccaaacctataagcaaaaaaatttcacattaattggcccgcaatcctattagtcttattatactagagtaggaaaaaaaacaattacacaacttgtcttattattctctatgctaatgaatatttttcccttttgttagaaatcagtgtttcctaatttattgagtattaattccactcaccgcatatatttaccgttgaataagaaaattttacacataattctttttaagataaataatttttttatactagatcttatatgattacgtgaagccaagtgggttatactaatgatatataatgtttgatagtaatcagtttataaaccaaatgcatggaaatgttacgtggaagcacgtaaattaacaagcattgaagcaaatgcagccaccgcaccaaaaccaccccacttcacttccacgtaccatattccatgcaactacaacaccctaaaacttcaataaatgcccccaccttcacttcacttcacccatcaatagcaagcggccgcaccatggcgatttccgatgagcctgaaagtgtagccactgctctcaaccactcttccctgcgccgccgtccctccgccacctccaccgccggcctcttcaattcgcctgagacaaccaccgacagttccggtgatgacttggccaaggattctggttccgacgactccatcaacaacgacgacgccgccgtcaattcccaacagcaaaacgaaaaacaagacactgatttctccgtcctcaaattcgcctaccgtccttccgtccccgctcaccgcaaagtgaaggaaagtccgctcagctccgacactattttccgtcagagtcacgcgggcctcttcaacctttgtatagtagtccttgttgctgtgaatagccgactcatcattgagaatttaatgaagtatggttggttgatcaaatctggcttttggtttagtgcaaagtcattgagagactggccccttttcatgtgttgtctttctcttgtggtatttcctttcgctgcctttatggtggagaagttggcacaacggaagtgtatacccgaaccagttgttgttgtacttcatataatcattacctcaacttcgcttttctatccagttttagttattctcaagtgtgattctgcttttgtatcaggtgtcacgttaatgctgttttcttgtgttgtatggttaaaattggtgtcttttgcacatacaaactatgatatgagagcacttaccaaattagttgaaaagggagaagcactgctcgatactctgaacatggagtatccttacaacgtaaccttcaagagcttggcatatttcctgcttgcccctacattatgttaccagccaagctatcctcgcacaccttatattcgaaagggttggttgtttcgccaacttgtcaagctgatagtatttacaggagttatgggatttataatagaacaatatattaatcccatagtacaaaattcacagcatcctctcaagggaaaccttctttacgccaccgagagagttctgaagctttctgttccaaatttatatgtgtggctctgcatgttctattgctttttccacctttggttaaatatcgtggcagagcttcttcgatttggtgatcgtgaattctacaaggattggtggaatgccaaaactgtcgaagattattggaggatgtggaatatgcctgttcacaaatggatgatccgccacctatattttccatgtttaaggcacggtctaccaaaggctgctgctcttttaatttccttcctggtttctgctttattccatgagctgtgcattgctgttccttgccacatgttcaagttgtgggctttcggtggaattatgtttcaggttcctttggtcttgatcactaattatctgcaaaataaattcaaaaactcaatggttggaaatatgattttttggttcatattcagtatcgttggtcaacctatgtgtgtactgctatactaccatgacttgatgaataggaaaggcaaacttgactgagcggccgcgaagttaaaagcaatgttgtcacttgtacgtactaacacatgatgtgatagtttatgctagctagctataacataagctgtctctgagtgtgttgtatattaataaagatcatcactggtgaatggtgatcgtgtacgtaccctacttagtaggcaatggaagcacttagagtgtgctttgtgcatggccttgcctctgttttgagacttttgtaatgttttcgagtttaaatctttgcctttgcgtacgtctttccacaatacataactattaattaatcttaaataaataaaggataaaatatttttttttcttcataaagttaaaatatgttattttttgtttagatgtatattcgaataaatctaaatatatgataatgattttttatattgattaaacatataatcaatattaaatatgatatttttttatataggttgtacacataattttataaggataaaaaatatgataaaaataaattttaaatatttttatatttacgagaaaaaaaaatattttagccataaataaatgaccagcatattttacaaccttagtaattcataaattcctatatgtatatttgaaattaaaaacagataatcgttaagggaaggaatcctacgtcatctcttgccatttgtttttcatgcaaacagaaagggacgaaaaaccacctcaccatgaatcactcttcacaccatttttactagcaaacaagtctcaacaactgaagccagctctctttccgtttctttttacaacactttctttgaaatagtagtattttttttcacatgatttattaacgtgccaaaagatgcttattgaatagagtgcacatttgtaatgtactactaattagaacatgaaaaagcattgttctaacacgataatcctgtgaaggcgttaactccaaagatccaatttcactatataaattgtgacgaaagcaaaatgaattcacatagctgagagagaaaggaaaggttaactaagaagcaatacttcagcggccgcatgccgaatcccgaggctcaccacccctcccggtcccgggcccggccctccacgtcagccgcggcccgccccccggcccgggcccgcgtctccctccgccagcttctgcgcgtggcatcagtcgcgagcggcatccagttcgggtgggccttacagctctctctgctgacgccctacgttcagcagctggggatcccccaccaatgggccagcatcatctggctctgcggcccagtctccggcctcttcgtgcagcccctcgtcggccacatgagcgaccgctgcaccagccgctacggccgccgcaggcccttcatcctcgtcggcgccgtcgccatcgtcgccgctgttctcgtcatcgcttacgccgccgacatcggctggctcctcggcgacaccgcggactaccgccctgccgccatcaccgtcttcatcgtcggcttctggatcctcgacgtcgctaacaacgtcacgcaaggtccctgccgtgccttgctcggtgatctcactagcaaggatcctcgaaggacacgtgttgcaaatgcttattactcactgtttatggccattggtaacattcttggctatgcaactggatcatatagtggttggtacaagatttttacttttgccctttcccctgcttgcacaattagttgtgcaaatctcaagtctgctttctttcttgacattgctttcattgcggtcacaacatatatcagcatcatggcagctcatgaagtgcctctaaattcaagtgaggcggcccatgctgaagcaggggcaggggagtcaggtagtgcagaagaagctttcatgtgggaattatttgggacattcaaatattttacaacccctgtatggataattctgtctgttactgctctgacatggattgggtggttcccatttactctctttgatactgattggatgggtcgagagatttatggtggtgatccaaatcaaggccttgtttatgatactggagttagaatgggagcacttggtttgttgcttaattcagttgttcttgcattaacatcattgttcatggagaggctatgcaggaagaggggagctggttttgtgtggggaatctcaaatatcatgatgaccgtttgctttcttgcaatgctagtagtaacctatgtggcaaataacatgggctatataggcaaagatttaccaccaactggcattgtgatagctgcgttgattatctttaccattcttgggtttccactggcaatcacttatagtgttccatatgccttaatttccacacatattgagtcattgggactcggccaagggttatcaatgggtgtcctaaatctggcaatagtggtcccacagataatagtgtcactgggaagtggaccatgggatcagctatttggtggaggaaactccccagcctttgctgtggcagctgtttcagcccttatcagtggactcatagctgtgttggctattcctcgatctggtgctcaaaaggctcgaagccatgtatgagcggccgcctgaacgggaattaaacctataaacataaatataaataatatatataaacctaagtgtctaagttccataaattaagctgtagtctctggcttaaaacatgttaggtttgtttatacaagtagttggatgtttggagtacttcggtcttttgcgtaccatcaatatttaagaactaagttagttatgttccgtaacttatgggctcttaattaaactatatctgcacaaaattatatatatatcaaatgtgatggtatgtggactataaaaagatatggttgagaaccacaaactttgaaacttcgaataatatattgccagtgacagtcttgttgatttgttatagcaagtcctattttcttaatcattgctttgttttaacgtacctagatttcataacttttgtctttgtctcaagctgaacctaatgatgatagtaatattaacttattgtataggggtatttcataggataaaaaatgatgtgcaattacgtgtagaccaaatattacttgatgacagatggcctgcaggatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgctagtcgactaagtcatcaactattccaagctacgtatttgggagtttgtggagtacagcaagatgatatacctagacggtgatatccaagtttttgacaacattgaccacttgtttgacttgcctgataactacttctatgcggtgatggactgtttctgtgagccaacttggggccacactaaacaatatcagatcggttactgccagcagtgcccccataaggttcagtggcccactcactttgggcccaaacctcctctctatttcaatgctggcatgtttgtgtatgagcccaatttggctacttaccgtgacctccttcaaacagtccaagtcacccagcccacttcctttgctgaacaggattttttgaacatgtacttcaaggacaaatataggccaattcctaatgtctacaatcttgtgctggccatgctgtggcgtcaccctgagaacgttgagcttgacaaagttaaagtggttcactactgtgctgctgggtctaagccttggaggtacactgggaagtgactcgaggtcatcaattactccaagctacgtatttgggagttcgtggagtacaagaagacgatatacctagacggtgacatccaagtatttggaaacatagaccacttgtttgatctgcctgataattatttctatgcggtgatggattgtttctgcgagaagacttggagccacacccctcagttccagattgggtactgccaacagtgccctgataaggttcaatggccctctcactttggttccaaacctcctctatatttcaatgctggcatgtttgtttatgagcctaatctcgacacctaccgtgatcttctccaaactgtccaactcaccaagcccacttcttttgctgagcaggactttctcaacatgtacttcaaggacaagtacaagccaataccgaacatgtacaaccttgtgctggccatgttgtggcgtcaccctgaaaatgttgaacttgataaagttcaagtggttcattactgtgctgctgggtctaagccttggaggttcactgggaagtaactgcaggtcatcaactactccaagctccgtatatgggagtttgtggagtacagcaagatgatatacttggacggagacattgaggtatatgagaacatagaccacctatttgacctacctgatggtaacttttacgctgtgatggattgtttctgcgagaagacatggagtcacacccctcagtacaaggtgggttactgccagcaatgcccggagaaggtgcggtggcccaccgaattgggtcagcccccttctctttacttcaacgctggcatgttcgtgttcgaacccaacatcgccacctatcatgacctattgaaaacggtgcaagtcaccactcccacctcgttcgctgaacaagatttcttgaacatgtacttcaaggacatttacaagccaatccctttaaattacaatcttgtcctcgccatgctgtggcgccacccggaaaacgttaaattagaccaagtcaaggttgttcactattgcgcagcggggtccaagccatggagatatacggggaagtagcctaggcgtacgcaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtcctaggctacttccccgtatatctccatggcttggaccccgctgcgcaatagtgaacaaccttgacttggtctaatttaacgttttccgggtggcgccacagcatggcgaggacaagattgtaatttaaagggattggcttgtaaatgtccttgaagtacatgttcaagaaatcttgttcagcgaacgaggtgggagtggtgacttgcaccgttttcaataggtcatgataggtggcgatgttgggttcgaacacgaacatgccagcgttgaagtaaagagaagggggctgacccaattcggtgggccaccgcaccttctccgggcattgctggcagtaacccaccttgtactgaggggtgtgactccatgtcttctcgcagaaacaatccatcacagcgtaaaagttaccatcaggtaggtcaaataggtggtctatgttctcatatacctcaatgtctccgtccaagtatatcatcttgctgtactccacaaactcccatatacggagcttggagtagttgatgacctgcagttacttcccagtgaacctccaaggcttagacccagcagcacagtaatgaaccacttgaactttatcaagttcaacattttcagggtgacgccacaacatggccagcacaaggttgtacatgttcggtattggcttgtacttgtccttgaagtacatgttgagaaagtcctgctcagcaaaagaagtgggcttggtgagttggacagtttggagaagatcacggtaggtgtcgagattaggctcataaacaaacatgccagcattgaaatatagaggaggtttggaaccaaagtgagagggccattgaaccttatcagggcactgttggcagtacccaatctggaactgaggggtgtggctccaagtcttctcgcagaaacaatccatcaccgcatagaaataattatcaggcagatcaaacaagtggtctatgtttccaaatacttggatgtcaccgtctaggtatatcgtcttcttgtactccacgaactcccaaatacgtagcttggagtaattgatgacctcgagtcacttcccagtgtacctccaaggcttagacccagcagcacagtagtgaaccactttaactttgtcaagctcaacgttctcagggtgacgccacagcatggccagcacaagattgtagacattaggaattggcctatatttgtccttgaagtacatgttcaaaaaatcctgttcagcaaaggaagtgggctgggtgacttggactgtttgaaggaggtcacggtaagtagccaaattgggctcatacacaaacatgccagcattgaaatagagaggaggtttgggcccaaagtgagtgggccactgaaccttatgggggcactgctggcagtaaccgatctgatattgtttagtgtggccccaagttggctcacagaaacagtccatcaccgcatagaagtagttatcaggcaagtcaaacaagtggtcaatgttgtcaaaaacttggatatcaccgtctaggtatatcatcttgctgtactccacaaactcccaaatacgtagcttggaatagttgatgacttagtcgactagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacctgcaggtcgactctagtaagctaaacaccggtgttaattaggtaatttcaccgcgtctagtgagctcggtacccgggtaccgg

SEQ ID NO: 7 - DNA, 3965 bp, synthetic construct
  misc_feature 1..3965: note = synthesized DNA of PHP17522A
  source 1..3965: mol_type = other DNA
ggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgtttgactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatattcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtac

SEQ ID NO: 8 - DNA, 6270 bp, synthetic construct
  misc_feature 1..6270: note = synthesized DNA of PHP17734A
  source 1..6270: mol_type = other DNA
cgcgccaagcttggatccgcccgatccctcaattcttctaagataaaaaatttaacagcaatactttttaaacaaattcattcaaaattgttttgcaaattgcatttgataaataatttaatcaagtacttacgccacaccaacttacaacaatgtcatacataaatcatagtgtgacattattgcgatttttgtactgaaaaataatatttttaaaatatatgtacgaaggcaaagagctaaactttgttgttctatctctatttcaaattcttcctttccatctctcttttttcttttcaattcacccttcacattctctttcaattgaggaatggttcaacactacgtgcgataggctaaatgtcacttccactttaatataaataaaggatcatattcttgtatcaattgataaagaaagttttttttttcttcatgtttttatctgcctctaactactagtaagtgctattaattagagcttaagttgcatagaattaaagagaaacatttgagagttgagagatgattagcaataattttaatcaataactttaatcaataacttttagtatatttcgcatttgatttaactttttattatcctttttcaaattattctttcaaaatgatatcattttaaatattaatacaaatcttaacatcatttggaagggataacggagagacaatttggaagggataagagaagtcaatttcatccccaattagattaatcgaccgtttatgtaagcccctattgcacgagtggttgattgccacgtgtccctaacactgtgttgaagctcgttgcaaacagacacgcggcaattacgtgtaagacgattagtccaataatcctcagaaacttgccacgcgtactgcactgacacgtgtgcaaaagatagcgccgcacctaaatctatttatttggtagcatgcggtgtgctgttgaaagaagaaagaacctaagtgagaaacaaagaaaggaaataattgatctttgaaaatgcagggaggaaagaaagctggaggcggccgggctagagcggccggagctggtcatctcgctcatcgtcgagtcggcggccggagctggtcatctcgctcatcgtcgagtcggcggccgctgagctgatttaaatcaccactgtcaaaaccaccatcaccgacgctcaagccaaggtcgccaccgatcatggtcgtgcctacgtcaccttcctcgccggaaacggtgactatgtgaaaggtgtcgttggcttggcaaaaggtctgagaaaagtgaagagcatgtaccctctggtggttgcagtgctacccgatgttccccaagatcaccgcaacattctcacctcccaaggttgcattgttagagagattgagcccgtgtaccccccagagaatcaaacccagtttgccatggcatattacgtcatcaactattccaagctacgtatttgggagtttgtggagtacagcaagatgatatacctagacggtgatatccaagtttttgacaacattgaccacttgggatcgatcctgagctgatttaaaccaccgttgttgccaatgtcaccaccgagcaattacccaaggctcgtggaggaagtgggcgtgccttcgtgacctttcttgctgggaacggtgattacgtaaagggtgtcgtgggtttggccaaaggactgagaaaggccaaaagcatgtaccctttggtggttgctgtgttaccagatgttcctgaagaacatcgtgagattctcaaatcccaaggttgcattgtcagggagattgaacctgtgtaccctcctgagaaccagacccagttcgtcatggcctattatgtcatcaattactccaagctacgtatttgggagttcgtggagtacaagaagacgatatacctagacggtgacatccaagtatttggaaacatagaccacttgtttgatctgtgagctgatttaagcggccgccgactcgacgatgagcgagatgaccagctccggccgccgactcgacgatgagcgagatgaccagctccggccgcaagtatgaactaaaatgcacgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaaggaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtaaagcaatggttgaccgtgtgcttagcttcttttattttatttttttatcagcaaagaataaataaaataaaatgagacacttcagggatgtttcaacggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcggccgctgagtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattaagcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacgg

SEQ ID NO: 9 - DNA, 6638 bp, synthetic construct
  misc_feature 1..6638: note = synthesized DNA of PHP29252A
  source 1..6638: mol_type = other DNA
cgcgccaagcttggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccgctagtcgactaagtcatcaactattccaagctacgtatttgggagtttgtggagtacagcaagatgatatacctagacggtgatatccaagtttttgacaacattgaccacttgtttgacttgcctgataactacttctatgcggtgatggactgtttctgtgagccaacttggggccacactaaacaatatcagatcggttactgccagcagtgcccccataaggttcagtggcccactcactttgggcccaaacctcctctctatttcaatgctggcatgtttgtgtatgagcccaatttggctacttaccgtgacctccttcaaacagtccaagtcacccagcccacttcctttgctgaacaggattttttgaacatgtacttcaaggacaaatataggccaattcctaatgtctacaatcttgtgctggccatgctgtggcgtcaccctgagaacgttgagcttgacaaagttaaagtggttcactactgtgctgctgggtctaagccttggaggtacactgggaagtgactcgaggtcatcaattactccaagctacgtatttgggagttcgtggagtacaagaagacgatatacctagacggtgacatccaagtatttggaaacatagaccacttgtttgatctgcctgataattatttctatgcggtgatggattgtttctgcgagaagacttggagccacacccctcagttccagattgggtactgccaacagtgccctgataaggttcaatggccctctcactttggttccaaacctcctctatatttcaatgctggcatgtttgtttatgagcctaatctcgacacctaccgtgatcttctccaaactgtccaactcaccaagcccacttcttttgctgagcaggactttctcaacatgtacttcaaggacaagtacaagccaataccgaacatgtacaaccttgtgctggccatgttgtggcgtcaccctgaaaatgttgaacttgataaagttcaagtggttcattactgtgctgctgggtctaagccttggaggttcactgggaagtaactgcaggtcatcaactactccaagctccgtatatgggagtttgtggagtacagcaagatgatatacttggacggagacattgaggtatatgagaacatagaccacctatttgacctacctgatggtaacttttacgctgtgatggattgtttctgcgagaagacatggagtcacacccctcagtacaaggtgggttactgccagcaatgcccggagaaggtgcggtggcccaccgaattgggtcagcccccttctctttacttcaacgctggcatgttcgtgttcgaacccaacatcgccacctatcatgacctattgaaaacggtgcaagtcaccactcccacctcgttcgctgaacaagatttcttgaacatgtacttcaaggacatttacaagccaatccctttaaattacaatcttgtcctcgccatgctgtggcgccacccggaaaacgttaaattagaccaagtcaaggttgttcactattgcgcagcggggtccaagccatggagatatacggggaagtagcctaggtgagctgatttaagatttatcaaaagttggggttacaaattttggaagcttcagtgtggaagtaatagacccagtttctgactatctggagctattggagacagtatttgattttcagctaatcagaggtcttctttcacgtccagattttaggtttatatttgatgccatgcatgcagttactggtgcttatgctaaacccatcttcgttgataaactcggtgctagtctggattcaatttcaaatggaatccctttggaagattttggacatggccatcctgatcctaatctaacatatgcgaaggatcttgtcgacattctgtatgctgaaaatggacctgattttggagctgccagtgatggggatggtgatagaaatatgattttaggaagaagtttctttgtaactccttcagactctgtagcagttattgcagccaatgcaagagaagcgattccatacttcaagaacggtgttaagggtcttgctcgatcaatgccaacaagcggtgctctggaccgtgctgctaaaaaattgaacctccctttctgagctgatttaacgtacgcaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtcctgcaggttaaatcagctcagaaagggaggttcaattttttagcagcacggtccagagcaccgcttgttggcattgatcgagcaagacccttaacaccgttcttgaagtatggaatcgcttctcttgcattggctgcaataactgctacagagtctgaaggagttacaaagaaacttcttcctaaaatcatatttctatcaccatccccatcactggcagctccaaaatcaggtccattttcagcatacagaatgtcgacaagatccttcgcatatgttagattaggatcaggatggccatgtccaaaatcttccaaagggattccatttgaaattgaatccagactagcaccgagtttatcaacgaagatgggtttagcataagcaccagtaactgcatgcatggcatcaaatataaacctaaaatctggacgtgaaagaagacctctgattagctgaaaatcaaatactgtctccaatagctccagatagtcagaaactgggtctattacttccacactgaagcttccaaaatttgtaaccccaacttttgataaatcttaaatcagctcacctaggctacttccccgtatatctccatggcttggaccccgctgcgcaatagtgaacaaccttgacttggtctaatttaacgttttccgggtggcgccacagcatggcgaggacaagattgtaatttaaagggattggcttgtaaatgtccttgaagtacatgttcaagaaatcttgttcagcgaacgaggtgggagtggtgacttgcaccgttttcaataggtcatgataggtggcgatgttgggttcgaacacgaacatgccagcgttgaagtaaagagaagggggctgacccaattcggtgggccaccgcaccttctccgggcattgctggcagtaacccaccttgtactgaggggtgtgactccatgtcttctcgcagaaacaatccatcacagcgtaaaagttaccatcaggtaggtcaaataggtggtctatgttctcatatacctcaatgtctccgtccaagtatatcatcttgctgtactccacaaactcccatatacggagcttggagtagttgatgacctgcagttacttcccagtgaacctccaaggcttagacccagcagcacagtaatgaaccacttgaactttatcaagttcaacattttcagggtgacgccacaacatggccagcacaaggttgtacatgttcggtattggcttgtacttgtccttgaagtacatgttgagaaagtcctgctcagcaaaagaagtgggcttggtgagttggacagtttggagaagatcacggtaggtgtcgagattaggctcataaacaaacatgccagcattgaaatatagaggaggtttggaaccaaagtgagagggccattgaaccttatcagggcactgttggcagtacccaatctggaactgaggggtgtggctccaagtcttctcgcagaaacaatccatcaccgcatagaaataattatcaggcagatcaaacaagtggtctatgtttccaaatacttggatgtcaccgtctaggtatatcgtcttcttgtactccacgaactcccaaatacgtagcttggagtaattgatgacctcgagtcacttcccagtgtacctccaaggcttagacccagcagcacagtagtgaaccactttaactttgtcaagctcaacgttctcagggtgacgccacagcatggccagcacaagattgtagacattaggaattggcctatatttgtccttgaagtacatgttcaaaaaatcctgttcagcaaaggaagtgggctgggtgacttggactgtttgaaggaggtcacggtaagtagccaaattgggctcatacacaaacatgccagcattgaaatagagaggaggtttgggcccaaagtgagtgggccactgaaccttatgggggcactgctggcagtaaccgatctgatattgtttagtgtggccccaagttggctcacagaaacagtccatcaccgcatagaagtagttatcaggcaagtcaaacaagtggtcaatgttgtcaaaaacttggatatcaccgtctaggtatatcatcttgctgtactccacaaactcccaaatacgtagcttggaatagttgatgacttagtcgacgg

SEQ ID NO: 10 - DNA, 3984 bp, synthetic construct
  misc_feature 1..3984: note = synthesized DNA of PHP19031A
  source 1..3984: mol_type = other DNA
cgcgccaagcttggatcctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtccaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgaccgatccgtcgacgg

SEQ ID NO: 11 - DNA, 3162 bp, synthetic construct
  misc_feature 1..3162: note = synthesized DNA of PHP29882A
  source 1..3162: mol_type = other DNA
cgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccctaggtgagctgatttaagatttatcaaaagttggggttacaaattttggaagcttcagtgtggaagtaatagacccagtttctgactatctggagctattggagacagtatttgattttcagctaatcagaggtcttctttcacgtccagattttaggtttatatttgatgccatgcatgcagttactggtgcttatgctaaacccatcttcgttgataaactcggtgctagtctggattcaatttcaaatggaatccctttggaagattttggacatggccatcctgatcctaatctaacatatgcgaaggatcttgtcgacattctgtatgctgaaaatggacctgattttggagctgccagtgatggggatggtgatagaaatatgattttaggaagaagtttctttgtaactccttcagactctgtagcagttattgcagccaatgcaagagaagcgattccatacttcaagaacggtgttaagggtcttgctcgatcaatgccaacaagcggtgctctggaccgtgctgctaaaaaattgaacctccctttctgagctgatttaacgtacgcaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtcctgcaggttaaatcagctcagaaagggaggttcaattttttagcagcacggtccagagcaccgcttgttggcattgatcgagcaagacccttaacaccgttcttgaagtatggaatcgcttctcttgcattggctgcaataactgctacagagtctgaaggagttacaaagaaacttcttcctaaaatcatatttctatcaccatccccatcactggcagctccaaaatcaggtccattttcagcatacagaatgtcgacaagatccttcgcatatgttagattaggatcaggatggccatgtccaaaatcttccaaagggattccatttgaaattgaatccagactagcaccgagtttatcaacgaagatgggtttagcataagcaccagtaactgcatgcatggcatcaaatataaacctaaaatctggacgtgaaagaagacctctgattagctgaaaatcaaatactgtctccaatagctccagatagtcagaaactgggtctattacttccacactgaagcttccaaaatttgtaaccccaacttttgataaatcttaaatcagctcacctagggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacgg

SEQ ID NO: 12 - DNA, 9744 bp, synthetic construct
  misc_feature 1..9744: note = synthesized DNA of PHP29959A
  source 1..9744: mol_type = other DNA
cgcgccggtaccgggccccccctcgaggtcgacggtatcgataagcttgatatcgaattcctgcagcccgggggatccactagttctagagcggcccgcgccgtcgacggatataatgagccgtaaacaaagatgattaagtagtaattaatacgtactagtaaaagtggcaaaagataacgagaaagaaccaatttctttgcattcggccttagcggaaggcatatataagctttgattattttatttagtgtaatgatttcgtacaaccaaagcatttatttagtactctcacacttgtgtcgcggccgctagtcgactaagtcatcaactattccaagctacgtatttgggagtttgtggagtacagcaagatgatatacctagacggtgatatccaagtttttgacaacattgaccacttgtttgacttgcctgataactacttctatgcggtgatggactgtttctgtgagccaacttggggccacactaaacaatatcagatcggttactgccagcagtgcccccataaggttcagtggcccactcactttgggcccaaacctcctctctatttcaatgctggcatgtttgtgtatgagcccaatttggctacttaccgtgacctccttcaaacagtccaagtcacccagcccacttcctttgctgaacaggattttttgaacatgtacttcaaggacaaatataggccaattcctaatgtctacaatcttgtgctggccatgctgtggcgtcaccctgagaacgttgagcttgacaaagttaaagtggttcactactgtgctgctgggtctaagccttggaggtacactgggaagtgactcgaggtcatcaattactccaagctacgtatttgggagttcgtggagtacaagaagacgatatacctagacggtgacatccaagtatttggaaacatagaccacttgtttgatctgcctgataattatttctatgcggtgatggattgtttctgcgagaagacttggagccacacccctcagttccagattgggtactgccaacagtgccctgataaggttcaatggccctctcactttggttccaaacctcctctatatttcaatgctggcatgtttgtttatgagcctaatctcgacacctaccgtgatcttctccaaactgtccaactcaccaagcccacttcttttgctgagcaggactttctcaacatgtacttcaaggacaagtacaagccaataccgaacatgtacaaccttgtgctggccatgttgtggcgtcaccctgaaaatgttgaacttgataaagttcaagtggttcattactgtgctgctgggtctaagccttggaggttcactgggaagtaactgcaggtcatcaactactccaagctccgtatatgggagtttgtggagtacagcaagatgatatacttggacggagacattgaggtatatgagaacatagaccacctatttgacctacctgatggtaacttttacgctgtgatggattgtttctgcgagaagacatggagtcacacccctcagtacaaggtgggttactgccagcaatgcccggagaaggtgcggtggcccaccgaattgggtcagcccccttctctttacttcaacgctggcatgttcgtgttcgaacccaacatcgccacctatcatgacctattgaaaacggtgcaagtcaccactcccacctcgttcgctgaacaagatttcttgaacatgtacttcaaggacatttacaagccaatccctttaaattacaatcttgtcctcgccatgctgtggcgccacccggaaaacgttaaattagaccaagtcaaggttgttcactattgcgcagcggggtccaagccatggagatatacggggaagtagcctaggacctgcacatcaccatgttttggtcatatattagaaaagttataaattaaaatatacacacttataaactacagaaaagcaattgctatatactacattcttttattttgaaaaaaatatttgaaatattatattactactaattaatgataattattatatatatatcaaaggtagaagcagaaacttacctgcgtacgcctaggctacttccccgtatatctccatggcttggaccccgctgcgcaatagtgaacaaccttgacttggtctaatttaacgttttccgggtggcgccacagcatggcgaggacaagattgtaatttaaagggattggcttgtaaatgtccttgaagtacatgttcaagaaatcttgttcagcgaacgaggtgggagtggtgacttgcaccgttttcaataggtcatgataggtggcgatgttgggttcgaacacgaacatgccagcgttgaagtaaagagaagggggctgacccaattcggtgggccaccgcaccttctccgggcattgctggcagtaacccaccttgtactgaggggtgtgactccatgtcttctcgcagaaacaatccatcacagcgtaaaagttaccatcaggtaggtcaaataggtggtctatgttctcatatacctcaatgtctccgtccaagtatatcatcttgctgtactccacaaactcccatatacggagcttggagtagttgatgacctgcagttacttcccagtgaacctccaaggcttagacccagcagcacagtaatgaaccacttgaactttatcaagttcaacattttcagggtgacgccacaacatggccagcacaaggttgtacatgttcggtattggcttgtacttgtccttgaagtacatgttgagaaagtcctgctcagcaaaagaagtgggcttggtgagttggacagtttggagaagatcacggtaggtgtcgagattaggctcataaacaaacatgccagcattgaaatatagaggaggtttggaaccaaagtgagagggccattgaaccttatcagggcactgttggcagtacccaatctggaactgaggggtgtggctccaagtcttctcgcagaaacaatccatcaccgcatagaaataattatcaggcagatcaaacaagtggtctatgtttccaaatacttggatgtcaccgtctaggtatatcgtcttcttgtactccacgaactcccaaatacgtagcttggagtaattgatgacctcgagtcacttcccagtgtacctccaaggcttagacccagcagcacagtagtgaaccactttaactttgtcaagctcaacgttctcagggtgacgccacagcatggccagcacaagattgtagacattaggaattggcctatatttgtccttgaagtacatgttcaaaaaatcctgttcagcaaaggaagtgggctgggtgacttggactgtttgaaggaggtcacggtaagtagccaaattgggctcatacacaaacatgccagcattgaaatagagaggaggtttgggcccaaagtgagtgggccactgaaccttatgggggcactgctggcagtaaccgatctgatattgtttagtgtggccccaagttggctcacagaaacagtccatcaccgcatagaagtagttatcaggcaagtcaaacaagtggtcaatgttgtcaaaaacttggatatcaccgtctaggtatatcatcttgctgtactccacaaactcccaaatacgtagcttggaatagttgatgacttagtcgactagcggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaagtgtgttattaacccttctcttcgaggatccaagcttggcgcgggccgccaccgcggtggggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatc

(19) United States
(12) Patent Application Publication
(10) Pub. No.: US 2024/0060886 A1
(43) Pub. Date: Feb. 22, 2024

(54) NON-DESTRUCTIVE ASSAY FOR SOYBEAN SEEDS USING NEAR INFRARED ANALYSIS

(71) Applicant: PIONEER HI-BRED INTERNATIONAL, INC., JOHNSTON, IA (US)

(72) Inventors: ROSS M ALLEN, DES MOINES, IA (US); JOHN D EVERARD, GRIMES, IA (US); MIN REN, JOHNSTON, IA (US)

(73) Assignee: PIONEER HI-BRED INTERNATIONAL, INC., JOHNSTON, IA (US)

(21) Appl. No.: 18/305,333

(22) Filed: Apr. 22, 2023

Related U.S. Application Data
(63) Continuation of application No. 17/655,190, filed on Mar. 17, 2022, now Pat. No. 11,668,651.
Application No. 17/655,190 is a continuation of application No. 16/490,261, filed on Aug. 30, 2019, now Pat. No. 11,307,139.
(60) Provisional application No. 62/466,575, filed on Mar. 3, 2017.

(51) Int. Cl. G01N 21/359; B07C 5/342; C12N 15/82; G01N 21/3563; G01N 21/84; G01N 33/02

(52) U.S. Cl. CPC G01N 21/359; B07C 5/3425; C12N 15/8245; C12N 15/8247; G01N 21/3563; G01N 21/84; G01N 33/025; G01N 2021/8466

(57) ABSTRACT

Disclosed are methods and systems for spectral imaging of soybean samples to accurately and non-destructively measure the amount of sucrosyl-oligosaccharide in the soybean samples. Populations containing modified and unmodified soybean seeds and having varying amounts of sucrosyl-oligosaccharides, oil or protein can be sorted and separated and further used in soybean processing or breeding.

### REFERENCE TO SEQUENCE LISTING SUBMITTED ELECTRONICALLY

The official copy of the sequence listing is submitted electronically via Patent Center as an XML formatted sequence listing with a file named BB2225_SequenceListing created on Oct. 30, 2023, and having a size of 149,262 bytes and is filed concurrently with the specification. The sequence listing comprised in this XML formatted document is part of the specification and is herein incorporated by reference in its entirety.

### BACKGROUND

Soybeans are the world's foremost provider of vegetable protein and oil. Soybean oil is used in food and industrial products. Soybean flakes remaining after the removal of oil can be processed into various edible soy protein products or used to produce soybean meal for animal feeds.

Soybean seed reserves can be repartitioned through plant research and breeding techniques. Analytical techniques facilitate research by permitting assessment of the composition of the soybean seed, soybean flakes and soybean meal.

### SUMMARY

Non-destructive methods for accurately measuring the amount of a sucrosyl-oligosaccharide, such as stachyose, or a combination of stachyose and raffinose, in a soybean seed are provided which include steps of directing near infrared light from a light source onto a soybean seed to form modified light from the soybean seed, receiving the modified light in an imaging device, and measuring the amount of a sucrosyl-oligosaccharide in the soybean seed based on the received modified light. The amount of the sucrosyl-oligosaccharide can be measured to an accuracy that is within 0.2 wt. %, 0.3 wt. %, 0.4 wt. %, 0.5 wt. %, 0.6 wt. %, 0.7 wt. %, 0.8 wt. %, 0.9 wt. %, 1 wt. %, 1.1 wt. %, 1.2 wt. %, 1.3 wt. %, 1.4 wt. %, or 1.5 wt. % of the amount measured using a standard reference analytical method. Following measurements, the seed can be optionally transported to a first or second location depending on whether the amount of sucrosyl-oligosaccharide measured is above or below a threshold value. The threshold value for stachyose can be selected to be, for example, 1 wt. %, 0.9 wt. %, 0.8 wt. %, 0.7 wt. %, 0.6 wt. %, 0.5 wt. %, 0.4 wt. %, 0.3 wt. %, 0.2 wt. % or 0.1 wt. %.

The methods can be used with a single seed or a plurality of seeds in a batch and the method steps can be repeated multiple times. The sucrosyl-oligosaccharide can be accurately measured in at least 80%, 85%, 90%, 95%, 96%, 97%, 98% or 99% of the measurements taken. The methods are non-destructive and preserve the viability of the seed or otherwise permit other compositional analyses or processing to be undertaken. For stachyose measurements, the imaging device can be calibrated using a plurality of soybean seeds having variable stachyose contents falling in a range that includes values of less than 0.1 wt. %, 0.2 wt. %, or 0.3 wt. % stachyose and more than 4.5 wt. %, 5 wt. % or 5.5 wt. % stachyose. In some embodiments, the seed is genetically modified to overexpress a diglyceride acyltransferase.

Provided are methods for measuring stachyose in a population of soybean seeds by directing near infrared light from a light source onto a first and second subsample of a population of soybean seeds to form a first and second modified light, which is received in an imaging device and used to measure the amount of a sucrosyl-oligosaccharide in the first and second subsamples. The first and second subsamples are separated when the amount of stachyose measured differs by at least 1 percentage point between the subsamples and are combined when the amount of stachyose differs by less than 0.2 percentage points between the first and second subsamples. The population can, for example include genetically modified and unmodified soybean seeds, such as a modified diacylglycerol transferase and the oil content of the modified beans may be at least 1 percentage point higher than of the unmodified soybean seeds.

Provided are methods for processing soybean seeds which have been genetically modified to contain high oil, high protein, or a combination thereof compared with unmodified soybean seeds which include the steps of directing near infrared light from a light source onto a sample comprising or being a soybean seed to form modified light from the soybean seed which is received in an imaging device and used to measure the amount of a sucrosyl-oligosaccharide, such as stachyose or a combination of stachyose and raffinose, in the sample. The method steps can be repeated for at least 10 samples and soybean seeds above a threshold value, which indicates high oil, high protein, or a combination thereof, can be separated from soybean seeds below the threshold value. The method is sufficiently robust such that at least 90% of the soybean seeds below the threshold value are modified soybean seeds or at least 90% of the seeds above the threshold value are unmodified soybean seeds. The amount of sucrosyl-oligosaccharide can be measured to an accuracy that is within 0.2 wt. %, 0.3 wt. %, 0.4 wt. %, 0.5 wt. %, 0.6 wt. %, 0.7 wt. %, 0.8 wt. %, 0.9 wt. %, 1 wt. %, 1.1 wt. %, 1.2 wt. %, 1.3 wt. %, 1.4 wt. %, or 1.5 wt. % of the amount measured using a standard reference analytical method. In some embodiments, at least one of the modified seeds that is separated is grown and crossed with the same or a different soybean plant to produce progeny seed. The progeny seed can be grown and crossed with another plant having a genetic modification, such as a recombinant construct incorporated into its genome, to produce further progeny seed, the genetic modification optionally providing one or more traits such as herbicide tolerance, disease resistance, insect resistance, increased grain yield, increased nutritional content, increased growth rate, enhanced stress tolerance, altered maturity. The method can include an initial step of separating the sample comprising the soybean seed from the plurality of seeds such as in an automated method. The method can include the step of measuring the amount of oil in the seed based on the received modified light.

Provided are methods for processing soybean seeds which include seeds genetically modified to contain increased oil and increased protein and unmodified soybean seeds. The methods include the steps of directing near infrared light from a light source onto a sample comprising a soybean seed to form modified light from the soybean seed which is received in an imaging device and used to measure the amount of a sucrosyl-oligosaccharide, such as stachyose or a combination of stachyose and raffinose, in the soybean seed. The method steps are repeated for at least 100 samples or seeds. The amount of sucrosyl-oligosaccharide measured below a threshold value indicates high oil and high protein in the soybean seed and the measurements taken are such that at least 90% of the soybean seeds below the threshold value are the modified soybean seeds or at least 90% of the seeds above the threshold value are the unmodified soybean seeds. The seeds above the threshold value can differ by a least 1 wt. % stachyose from the seeds below the threshold value. The seeds can be further processed for removal of oil and production of soy flakes or meal.

Provided are methods for measuring the amount of a sucrosyl-oligosaccharide, such as stachyose, or a combination of stachyose and raffinose, in soybean meal or soybean flakes. The methods include the steps of directing near infrared light from a light source onto a soybean meal sample to form modified light from the soybean meal sample which is received in an imaging device and used to measure the amount of a sucrosyl-oligosaccharide in the soybean meal sample. The amount of the sucrosyl-oligosaccharide can be measured to an accuracy that is within 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1 wt. %, 1.1 wt. %, 1.2 wt. %, 1.3 wt. %, 1.4 wt. %, or 1.5 wt. % of the amount measured using a standard reference analytical method.

### DETAILED DESCRIPTION

Systems and methods for the sampling of soybean seeds and measurement of soybean seed components are provided, which permit individual seed analysis, soybean meal, flake or powder analysis, or analysis of bulk seeds in an accurate, non-destructive and efficient manner. The term “soybean” refers to the species Glycine max, Glycine soja, or any species or line that is sexually cross compatible with Glycine max. Unless indicated to the contrary, seed as used herein means soybean seed. Following analysis, the soybean seeds can be grown and allowed to self or be crossed with genetically different soybean plants to produce progeny seed that can be used in a plant breeding program. The systems and methods further permit efficient processing of the soybeans according to their composition, such as to produce oil and protein flakes or meal. The analysis includes accurate measurement of one or more sucrosyl-oligosaccharides. A sucrosyl-oligosaccharide is generally understood to be a short-chain, non-digestible oligosaccharide such as stachyose, raffinose and verbascose. Because verbascose and other minor sucrosyl-oligosaccharides are present in very low amounts, as used in this application, a sucrosyl-oligosaccharide means one or more of stachyose and raffinose.

Destructive analytical methods for the measurement of seed components such as oil, fatty acids, protein and sucrosyl-oligosaccharides are those which directly measure the component including steps such as powdering the material, extraction of the sucrosyl-oligosaccharide, and detection of the amount or concentration of sucrosyl-oligosaccharide using chromatographic methods. Certain of these methods are accredited by professional associations (e.g., American Oil Chemists Society (AOCS); the American Association of Analytical Chemists (AOAC); American Association of Cereal Chemists (AACC) or international standards accreditation agencies, e.g., The Codex Alimentarius, International Organization for Standards (ISO) and the International Union of Pure and Applied Chemistry (IUPAC)). While accurate, such methods are time consuming and typically require a large sample size; for example, 60 g or more of whole soybeans, a portion of which may be used to determine the moisture contents of the samples, to enable compositional reporting on a defined moisture basis. For example, an accredited method for oil content is AOCS Official Method Ba 3-38 which gravimetrically measures the oil content of powdered seed material after extraction with petroleum ether. An example of an accredited method for protein content is AOAC 990.03 or AOCS Ba 4e-93 which determine the protein content of ground soybean powders by combustion analysis.

Destructive analytical methods for analysis of raffinose and stachyose can be based on methods that have been validated for quantitation of simple sugars in cereal products (e.g., AACC Method 80-04 Determination of Simple Sugars (fructose, glucose, sucrose, maltose and lactose) in Cereal Products-HPLC Method; AOAC Official Method 982.14 Glucose, Fructose, Sucrose, and Maltose in Presweetened Cereals; Black, L. T., and Glover, J. D., 1980. A Simple and rapid HPLC analysis of sugars in soybeans and the factors affecting their standardization. Journal of the American Oil Chemists Society 1980; 143. However, a harmonized cross-validated method is not available.

Methods, systems and devices provided herein measure the amount of one or more sucrosyl-oligosaccharides in a soybean seed using non-destructive methods to an accuracy that is representative of the amount measured using standard reference analytical methods. The term “accuracy” refers to the degree to which the result of a measurement, calculation, or specification conforms to the correct value, a standard, or reference value. Useful examples of such values which may be achieved are accuracies within at least 25%, 20%, 19%, 18%, 17%, 16%,15%, 14%, 13%, 12%, 11%, 10%, 9%, 8%, 7%, 6%, 5%, 4%, 3%, 2%, or 1% of the amount measured using a standard reference analytical method as described herein, such as by weight. The accurate measurements may be achieved for at least 75%, 80%, 85%, 90%, 95%, 96%, 97%, 98%, 99%, 99.5%, or 99.9% of a population of seeds, a plurality of seeds, a plurality of individual or a plurality of seed samples measured. The size of the population or plurality of seeds can be at least 1, 2, 3, 4, 5, 10, 15, 20, 25, 30, 35, 40, 45, 50, 60, 70, 80, 90, 100, 150, 200, 250, 300, 400, 500, 750, 1000, 2000, 3000, 5000, 10000, 100000, or 1,000,000 individual seeds or seed batches. The term “reference chemistry” refers to the benchmark values obtained for the measurements of the compositions analyzed herein, using standard reference analytical methods. As used herein, the “standard reference analytical method” used for measuring stachyose, raffinose or a combination thereof is a chromatographic (wet-chemistry) technique performed as follows. One of skill in the art will understand that certain substitutions in the components and steps used in the following methods may be made without affecting the results of the analysis:

### Grinding

Analysis is performed on soybeans, ground into fine powders with a particle size ranging between 0.5 to 0.9 mm. For single soybean seed, grinding is performed in Spex Certiprep 1/2 x 2″ polycarbonate vials with cap (cat #3116PC). A ⅜″ stainless steel ball bearing is used to pulverize the seed using a Spex Certiprep 2000 Geno/Grinder at 1500 strokes/min for three, 30 second bursts, with a 1-minute rest between each cycle. Samples are retained in the grinding vial at room temperature, in the dark, to minimize moisture loss prior to further analysis.

For bulk samples, approximately seventy-five gram batches of beans are ground in a Foss Knifetec 1095 grinder (commercially available from FOSS North America, Eden Prairie, MN). The grinding chamber is cooled prior to and during the process by a circulating chiller set to 14° C. Samples are ground for 6×10 second bursts using a standard rotor blade. At the conclusion of each 10 second grinder burst the chamber is opened and the powder in the chamber is loosened and any material adhering to the chamber wall is returned to the center of the chamber, using a small rubber spatula. After grinding the powders are quantitatively recovered from the chamber and transferred to plastic specimen cups fitted with airtight lids (Fisher Brand, part number 14828321) to ensure minimal moisture loss prior to analysis. The sample chamber and blade are cleaned thoroughly with a soft brush and pneumatic air prior to introduction of the next sample. Sample cups were stored at room temperature in the dark prior to further analysis. Ground samples were not sieved or otherwise treated prior to further analysis, this ensured that the sub-aliquot analyzed is fully representative of the original bulk sample and allows small subsamples to be used to provide data that is representative of the whole.

### Powder Moisture Content Determination

The moisture contents of the ground soybean powders are determined according to AOCS Official Method Ba 2a-38, which can be modified for small samples as described below. In order to standardize analytical results for moisture content, 100-200 mg samples are weighed (recorded to an accuracy of 0.1 mg) into 13×100 mm sample tubes (VWR part number 53283-800). The samples are placed in a forced draft oven, set to 130° C. for two hours and are then allowed to equilibrate to room temperature, in a desiccator, prior to reweighing. Moisture contents are calculated according to the following formula:

$$Moisture = \frac{\begin{matrix}( wt . tube + tissue as is - wt . tube ) - \\ ( wt . tube + tissue dry - wt . tube )\end{matrix}}{( wt . tube + tissue as is - wt . tube )} \times 100$$

Moisture contents are used to adjust analytical results to a common moisture content using the following formula:

$$Analyte at desired moisture content , \text{} \% = \frac{F ( 100 - \% moisture content desired )}{( 100 - \% moisture content of ground sample )}$$

Where F is the measured wt. % of the analyte in the ground sample.

### Extraction of Soluble Carbohydrates

Prior to carbohydrate analysis samples are de-fatted as follows: Weigh powdered sample (approximately 20-30 mg; to an accuracy of 0.1 mg) into 13×100 mm tube (with Teflon® lined cap; VWR (53283-800)) and record weight. Add 2 mL Heptane, vortex and place into an ultrasonic bath (VWR Scientific Model 750D) at 60° C. for 15 min at full sonification-power (˜360W). Centrifuge for 5 min at 1700×g at room temperature. Decant the supernatant to a clean 13×100 mm glass tube; this sample is used to determine fatty acid profiles of the extracted oil. Add 1 mL acetone to the de-fatted pellet, vortex mix to disperse the material into the acetone and dry in a SpeedVac (Thermo Fisher Scientific 275 Aiken Road, Ashville, NC 28804). To the dry pellet add 2 mL of 80% ethanol. Vortex to break up pellet as much as possible. Extract on sonicator for 15 min at 60° C. Centrifuge for 5 min at 1700×g. Transfer supernatant to a clean 13×100 mm tube. Repeat the ethanol extraction two more times, combining all of the supernatants. Add 100 μL of phenyl-β-D glucopyranoside internal standard (phenyl-β-D glucopyranoside stock 0.5000+/−0.0010 g in 100 ml water) to the combined supernatant. Dry the extract in the SpeedVac and analyze for soluble carbohydrates as described below. Add 1 ml acetone to the extracted pellets and dry in the SpeedVac.

### Starch Digestion and Extraction

The starch digestion is performed directly on the acetone dried pellets from soluble carbohydrate extraction. Add 100 units of α-Amylase (α-amylase; Heat Stable from Bacillus licheniformis Sigma-Aldrich A-4551) in 0.9 mL 50 mM MOPS (3-(N-Morpholino) propane sulfonic acid) buffer pH 7.0, containing 5 mM CaCl₂ and mix. Place tubes into a heating block at 90° C. for 75 minutes. Mix several times during hydrolysis. Allow the tubes to cool to room temperature and add 5 units of Amyloglucosidase (commercially available from Roche 11 202 367 001) in 0.6 mL of 285 mM acetate buffer, pH 4.5 and incubate in a reciprocating water bath at 55° C. for 15-18 hours. Remove rack of tubes and bring to room temperature. Add 4.5 mL of absolute ethanol to each tube, to attain a final ethanol concentration 80% and vortex mix. Extract on sonicator for 15 min at 60° C. Centrifuge 5 min at 1700×g and decant supernatant to a 13×100mm tube and immediately place tube in SpeedVac to reduce the volume. Extract pellet a further 2 times with 2 mL 80% ethanol, combining supernatant with above each time. Add 100 μL of phenyl-β-D glucopyranoside (see above) to the combined supernatant before it is fully dry. Once the extract in the SpeedVac is dry analyze for soluble carbohydrates as described below.

### Total Soluble Carbohydrate Derivatization and Analysis

The dried samples from the soluble and starch extractions described above along with sets of sugar standard mixtures (containing; pinitol, sorbitol, fructose, glucose, myo-inositol, sucrose, raffinose and stachyose; at 0, 0.05, 0.1, 0.5, 1.00, 2.00, and 3.00 mg/tube; each containing the same amount (0.5 mg) of phenyl-β-D glucopyranoside internal standard) were solubilized in 1 ml anhydrous pyridine (Sigma-Aldrich P57506) containing 30 mg/ml of hydroxylamine HCl (Sigma-Aldrich 159417). Samples were placed on an orbital shaker (350 rpm) overnight and were then heated for 1 hr (75° C.) with vigorous vortex mixing applied every 15 min. After cooling to room temperature 1 ml hexamethyldisilazane (Sigma-Aldrich H-4875) and 100 μL trifluoroacetic acid (Sigma-Aldrich T-6508) are added. The samples are vortex mixed and the precipitates are allowed to settle prior to transferring the supernatants to GC sample vials.

Samples are analyzed on an Agilent 6890 gas chromatography system fitted with a DB-17MS capillary column (30 m×0.32 mm×0.25 um film). Inlet and detector temperatures are both 275° C. After injection (2 μL, 20:1 split) the initial column temperature (150° C.) was increased to 180° C. at a rate 3° C./min and then at 25° C./min to a final temperature of 320° C. The final temperature is maintained for 10 min. Hydrogen gas is used as the carrier at a linear velocity of 51 cm/sec. Detection is by flame ionization. A 1 m length of plain 0.320 mm capillary tube (Agilent; 160-2325-5) is inserted between the inlet and the analytical column to act as a guard column. The two column sections are connected using a push-fit connector. Prior to all analytical runs three injections of a standard mixture containing 3 mg of each sugar is made to passivate the chromatography system. This process was found to enable full recovery of stachyose from the analytical samples, especially as the column aged. Ultra-Inert Inlet Liners (Agilent; 5190-3164) are used and are routinely changed based on indications of loss in stachyose recovery from the lowest concentration standard.

Data analysis is performed using Agilent ChemStation software. Each sugar is quantified relative to its own calibration curve, after dividing each individual peak by the area of the internal standard in each sample and standard. Final carbohydrate concentrations are expressed corrected for moisture content (see above). Residual sucrose, raffinose and stachyose recovered in the starch digestions are included in the total values reported for each sugar.

Soybean samples with a wide range in the amount sucrosyl-oligosaccharide, such as raffinose, stachyose, or a combination thereof can be accurately measured using these standard reference chemistry methods, facilitating the development spectroscopic techniques for accurate non-destructive measurements.

As used herein, the standard reference analytical method used for measuring the moisture content of whole beans is AOCS Official Method Ac 2-41, which measures the weight loss of a sample after a defined period in a forced draft oven heated to 130° C.

As used herein, the standard reference analytical method used for measuring the moisture content of soybean powders is AOCS Official Method Ba 2a-38, which measures the weight loss of a sample after a defined period in a forced draft oven heated to 130° C.

As used herein, the standard reference analytical method used for measuring oil is AOCS Official Method Ba 3-38 which gravimetrically measures the oil content of powdered seed material after extraction with petroleum ether.

As used herein, the standard reference analytical method used for measuring protein content is AOCS Ba 4e-93 which determines the protein content of ground soybean powders by combustion analysis.

As used herein, the standard reference analytical method used for measuring PROIL is the addition of the oil and protein contents determined by the standard reference analytical methods defined above.

As used herein, the standard reference analytical method used for determining fatty acid profiles is AOCS Official Method Ce 1e-91 on methyl esters derived from oil samples extracted from soybean powders.

The amount of sucrosyl-oligosaccharide such as stachyose, raffinose or a combination thereof in intact, whole, single or pooled soybean seeds can be measured using optical interrogation devices employing near infrared spectroscopy to an amount that is within at least 15%, 14%, 13%, 12%, 11%, 10%, 9%, 8%, 7%, 6%, 5%, 4%, 3%, 2%, or 1% of the amount measured using the standard reference analytical method described herein. Accuracy can be contrasted with precision, which refers to the closeness of two or more measurements to each other. Accurate and precise measurements of sucrosyl-oligosaccharides are achievable according to methods described herein. Precision with respect to the composition of soybean seed sample under analysis refers how closely replicate measurements of the same sample result in similar concentration or amounts being measured each time. Accuracy, with respect to the composition of soybean seed sample under analysis refers to the measured concentration or measured amount of the component of interest being similar to or the same as that obtained when running the standard reference analytical method on the same sample.

The accuracy obtained using the methods described herein is reproducible across multiple seeds or seed samples and facilitates high-throughput assessment on the composition of soybean seeds. For example, if a population of at least 10, 20, 50, 100, 250, 500, 1,000, 5,000, 10,000, 1,000,000, or 1,000,000 soybean seeds are measured using individual single-seed analysis, the amount of raffinose or stachyose may be accurately determined to within parameters described herein for at least 75%, 80%, 85%, 90%, 91%, 92%, 93%, 94%, 95%, 96%, 97%, 98%, 99%, 99.5% or 99.9% of the population of seeds.

Near Infrared Spectroscopy (NIRS) is a non-destructive tool for analyzing seed composition, with measurements based on the absorption of light energy (about 780 to 2500 nm) by H2O, C—C, C—H, O—H, N—H, S—H and C═O bonds in the organic constituents of the materials being analyzed. The present disclosure provides methods which are based on NIRS and on the absorption of light energy, in the near-infrared spectrum range (780 to 2500 nm), in the organic constituents of the materials being analyzed. Near infrared reflectance (NIR) and near infrared transmittance (NIT) light spectra can be collected and used. For example, methods described herein can be carried out as single-seed NIR (SS-NIR), bulk NIT or FT-NIR. The absorption of the light energy is proportional to the concentration of the constituent of interest and the modified light comprising one or more of transmitted and reflected light spectra from the seed can be converted to accurately measure the amounts or concentrations of the constituent of interest, such as a sucrosyl-oligosaccharide. “Modified light” as used in the context of this disclosure means light that is transmitted (transmitted light) and/or reflected (reflected light) from a seed or other object such as soybean meal or defatted soybean flakes after receiving light from a light source. Transflected light is a combination of reflected and transmitted light and is included in modified light.

In some embodiments, such as when single seed NIR (SS-NIR) is used, a suitable spectral range for a sucrosyl-oligosaccharide such as stachyose includes one or more values at or about 850 nm, 866 nm, 880 nm, 890 nm, 902 nm, 910 nm, 920 nm, 930 nm, 944 nm, 952 nm, 964 nm, 978 nm, 990 nm, 1004 nm, 1016 nm, 1032 nm, and 1042 nm, such as one or more values falling within 850-852 nm, 862-868 nm, 876-884 nm, 888-892 nm, 900-904 nm, 908-912 nm, 918-922 nm, 930-934 nm, 940-944 nm, 950-954 nm, 962-968 nm, 976-982 nm, 988-996 nm, 1000-1008 nm, 1012-1020 nm, 1026-1036 nm and 1040-1046 nm. In some embodiments, such as when FT-NIR is used, a suitable spectral region for a sucrosyl-oligosaccharide such as stachyose includes values at or about 1157-1283 nm and 1437-2254 nm. In some embodiments, such as when NIT is used for whole soybeans, a suitable spectral range for a sucrosyl-oligosaccharide such as stachyose includes values at or about 918, 930, 940, 950, 964, 980, and 996 nm.

In some embodiments, spectrometers are used to collect spectra from samples of soybeans, such as single seeds (e.g., SS-NIR), batches of seed from a single plant (e.g., FT-NIR), bulk samples from a field plot (e.g., NIT) or protein compositions such as protein meal and defatted soybean flakes (NIR). Protein meal can be produced by extracting oil from dried cleaned soybeans to produce dried defatted soy flakes, and processing the defatted soy flakes to produce soybean meal. Measurements taken are compared to the standard reference analytical method for samples sizes (single seeds or bulk samples). In some embodiments a diverse array of soybean samples grown in different seasons and different environments that display a wide range in the concentrations of the components are used to generate calibrations that provide for reliable and accurate measurements of the components.

In the methods provided, the conversion from modified light spectra from the soybean to the concentration of the constituent of interest is determined by a referencing to spectra from seeds where the constituent of interest has been measured using the standard reference analytical method for the component of interest as disclosed herein. Interpreting the near infrared spectral region (780-2500 nm) of seeds is complex for a number of reasons. Absorption in this region contains weaker overtones or harmonics of the fundamental frequencies and in combination bands, where absorption occurs in two or more overlapping fundamental bond energies. The energy absorption and resulting spectra are therefore composite vibrational signals of all of the resonating bonds within the organic components and water in the seed being analyzed. The spectral signal from any specific component is deciphered from the background and is influenced by the matrix that it is embedded in. For example, the molecular specific signal within an intact seed can be influenced by the environment such as geographic location, growing season, storage conditions and conditions during measurement, the genetic background, and the presence of similar molecules.

In some embodiments, accurate measurements of sucrosyl-oligosaccharides in a seed are achieved by utilizing a broad array of samples in which compounds with similar chemical compositions, such as sucrose, stachyose and raffinose, differ in a reciprocal manner. The sucrosyl-oligosaccharide specific signal is detected in the seed by having the sucrosyl-oligosaccharide present in a reciprocal concentration series of related molecules and visa-versa. A collection of mature soybean seeds that have significant differences in the concentrations of the sucrosyl-oligosaccharides facilitates this approach. The amount or concentration of sucrosyl-oligosaccharides can also be measured in seeds having the same or similar genetics that have been grown in multiple environments and over multiple seasons.

The measurements may be taken at any moisture content of the soybean. The moisture content of the soybean affects the weight percentages of components of the soybean, with drier beans generally having a higher weight percent of the component, such as oil, protein or sucrosyl-oligosaccharide. When comparing NIR-based measurements with standard reference analytical methods, measurements may be taken in each case at the same moisture content of soybean, or if measurements are taken at different moisture contents, the values obtained can be corrected to the same moisture content. Measurements can, for example, be taken at or standardized to a moisture content by weight of at least or at least about 0.01%, 0.1%, 0.5%, 1%, 2%, 3%, 4%, 5%, 6%, 7%, 8%, 9%, 10%, 11%, 12%, 13%, 14%, 15% or 20% and less than or less than about 35%, 30%, 25%, 20%, 19%, 18%, 17%, 16%, 15%, 14%, 13%, 12%, 11%, 10%, 9% or 8%. Unless indicated to the contrary, measurements described herein are at or about 13% by weight moisture content.

Provided are accurate non-destructive analytical NIRS-based methods for accurately measuring soluble carbohydrates such as sucrose and the sucrosyl-oligosaccharides raffinose and stachyose, which are appropriate for both single soybeans as well as bulk batches of soybeans and soybean meal. Following measurement, the seed remains viable and can be crossed with the same or different plant such as in a plant breeding program to produce progeny seed or processed for extraction of components such as oil and protein meal.

In the methods and systems provided, the amount or concentration of one or more sucrosyl-oligosaccharides in the seed can be accurately and non-destructively measured by interrogating a seed using an optical interrogation device, such as a spectrophotometer, which directs near-infrared light onto the seed, and using the reflected, transmitted or transflected (a combination of transmitted and reflected light) light spectra from the seed and detected by the optical interrogation device in combination with measurements used to generate calibration models obtained from previously assayed seed. In some embodiments, the seed is sorted or selected based on the amount or concentration of sucrosyl-oligosaccharide present in the seed. The seed can be a whole seed, an intact seed, a viable seed, an individual seed or a population of individual, whole, viable or intact seeds. In some embodiments, sorting decisions can be made following measurement and analysis of a single seed or following measurement and analysis of a population of seeds or a defined number of seeds assayed together in a seed sample. When a number of seeds are assayed together, an average for the measured values across the population of seeds may be obtained either by pooling the data collected from individual seeds from that population or by using methods in which a pooled sample of the soybean seeds are measured simultaneously. Following analysis, the seed remains viable and may be planted and grown to produce a soybean plant. The seeds remain whole, intact or viable before and following the analysis process. Protein meal from soybeans can be similarly analyzed.

By plotting values from the standard reference analytical method against the measurements taken by non-destructive analysis for a particular constituent, the R² value can be used to indicate the proportion of the data that is accounted for by an ideal line plotted through it. A value of 1 indicates highest accuracy. The root mean square error of calibration (RMSEC) indicates the resolving power of the measured values and can give an indication of the statistical confidence as to whether two values differ significantly from each other. Typically, values differing by 2×the RMSEC differ from each other at the 95% confidence level. The root mean square error of cross validation (RMSECV) is another statistical parameter that is used to assess the quality of the calibrations Once a model (calibration) is created, the data for a group of samples are removed and the influence of their omission is assessed. In robust models the RMSECV is similar to RMSEC. The cross validation also allows aberrant values which may incorrectly influence a model to be identified for further analysis. The RMSECV also gives an estimate of the potential of the models to predict the composition of samples outside the range represented in the calibration set.

Using the methods disclosed herein seeds, such as unmodified seeds and modified seeds in one or more populations of seeds, differing in stachyose content by at least 0.5%, 1.0%, 1.25%, 1.5%, 1.75%, 2.0%, 2.5% or 3.0% and less than 6.0%, 5.0%, 4.0%, 3.5%, 3.0% 2.5%, 2.0% or 1.5% (values are percentage points by weight) can be correctly identified for at least or at least about 80%, 85%, 90%, 91%, 92%, 93%, 94%, 95%, 96%, 97% or 98% of the population of seeds containing differing stachyose content.

The term “percentage point” (pp) difference, change, increase or decrease refers to the arithmetic difference of two percentages, e.g. [transgenic or genetically modified value (%)−control value (%)]=percentage points. For example, a modified seed may contain 20% by weight of a component and the corresponding unmodified control seed may contain 15% by weight of that component. The difference in the component between the control and transgenic seed would be expressed as 5 percentage points.

“Percent increase” or “percent decrease” refers to a change or difference expressed as a fraction of the control value, e.g. {[modified/transgenic/test value (%)−control value (%)]/control value (%)}×100%=percent change, or {[value obtained in a first location (%)−value obtained in second location (%)]/value in the second location (%)}×100=percent change. The term “total fatty acid content” refers to the sum of the five major fatty acid components found in soybeans, namely C16:0, C18:0, C18:1, C18:2, and C18:3. The term “total polyunsaturated fatty acid content” refers to the total C18:2 plus C18:3 content. The term “total saturated fatty acid content” refers to the total C16:0 plus C18:0 content.

Using the methods disclosed herein seeds, such as unmodified seeds and modified seeds, differing in raffinose content by at least 0.1%, 0.2%, 0.3%, 0.4%, 0.5%, 0.6%, 0.7%, 0.8%, 0.9%, 1% or 1.5% and less than 3%, 2.5%, 2%, 1.5%, 1%, 0.5%, 0.3% or 0.2% (values are percentage points by weight) in one or more populations of seeds can be correctly identified for at least or at least about 80%, 85%, 90%, 91%, 92%, 93%, 94%, 95%, 96%, 97% and 98% of the population of seeds.

The population can include at least or at least about 2, 5, 10, 15, 20, 25, 30, 35, 40, 25 35, 50, 55, 60, 65, 70, 75, 80, 85, 90, 95, 100, 125, 150, 175, 200, 250, 300, 350, 400, 450, 500, 600, 750, 1000, 5,000, 10,000 or 50,000 seeds and less than or less than about 5,000,000, 1,000,000, 500,000, 400,000, 250,000,100,000, 50,000, 10,000, 5,000, 2,500, 1,000, 750, 600, 500, 400, 300, 200, 150, 100, 75, 50 or 25 seeds.

In some embodiments, single individual intact soybeans are analyzed one seed at a time, such as with single-seed NIR techniques (SS-NIR) utilizing reflected light, transmitted light or a combination thereof. Such methods are useful, for example, to identify an individual seed carrying the desired trait, such as a transgene, edited gene or mutant allele, which results in a desired composition. Following analysis according the methods described herein, the seed can be used in research and plant breeding programs. For example, the seed can be grown to produce a plant which is crossed with itself or another different plant to produce progeny seed.

In some embodiments, small bulk quantities of seed, such as the amount of seed harvested from a single soybean plant (about 50-300 seeds) which may be homozygous, are analyzed together. FT-NIR, which utilizes reflectance NIR, can be used as in methods described herein for soybean samples harvested from individual plants. Such methods are useful, for example, in assessing or ranking trait performance at the single plant level, and can be used to make selections of plants for their use in further research or breeding. Such assessments may be used in evaluations of transgenic events in controlled environment and field studies.

In some embodiments, bulk seed analysis (bulk NIT methods) are provided which typically require a mass of at least or at least about 100 g, 200 g, 250 g, 300 g, 350 g, or 400 g and less than or less than about 2000 g, 1000 g, 900 g, 800 g, 700 g or 500 g of sample. Such methods are useful, for example, in the analysis of seed grown in field test plots and yield trials or from a bulk harvest and the identification of modified seed from unmodified seed. Such methods can be used, for example, at grain receiving sites such as grain elevators to determine the composition and quality of seeds delivered and the value of the grain shipment. Such methods may include a step of sampling the seed using a sampling system such as AOCS Official Method Ac 1-45.

Commercial soybean cultivars are homozygous for most traits and may be modified to have additional traits introduced by backcrossing (e.g., introgression of the trait of interest can be achieved by crossing to a second line containing the trait and repeated backcrossing to the original line while selecting for the trait of interest), genetic modification, mutation or transformation. Such additional traits can include one or more transgenes or gene modifications which alter the composition of the soybean seed or provide other agronomic characteristics such as herbicide or insect resistance. Selection of lines can include, for example, selections based on one or more of the performance of the line which produced the seed being measured, determining the presence or absence of a transgene, mutation or genetic modification in the seed, and assessing whether a transgene, genetic modification or mutant gene or coding sequence has been inherited by a seed, for example, by introgression through crossing and breeding steps.

Soybean seeds used in the methods and systems described herein can be generated using one or more techniques disclosed herein that facilitate integration or expression of a target sequence in the plant or seed. Examples include one or more of a particle gun, Agrobacterium, single-site integration, CRISPR-Cas (clustered, regularly interspaced, short, palindromic repeats-Cas) technology, TALENs (transcription activator-like effector nucleases), zinc-finger proteins (ZNF) or combination thereof.

Modified seed means seed that contains a genetic modification that results in an alteration of the composition of the seed. Examples of altered composition includes one or more of an increase or decrease in oil, protein, one or more fatty acids, one or more amino acids, one or more sucrosyl-oligosaccharides, sucrose, one or more carbohydrates, cell wall polysaccharides, cell wall monosaccharide components, fiber, starch, fermentable starch, cellulose, biopolymers, pharmaceuticals, secondary compounds, metabolites and combinations thereof.

Examples of genetic modifications in modified seed include transformation, such as with a recombinant construct containing a target sequence of interest operably connected to heterologous promoter, natural or induced mutations, and genome editing which may encompass altering one or more soybean genomic DNA sequences or a pre-existing transgenic sequence including regulatory elements, coding and non-coding sequences. The modification can be a single nucleotide deletion, substitution, a full or partial gene deletion, or insertion or alteration of an enhancer sequence, such as a promoter or promoter element, to increase expression. Deletions may include deletion of one or more exon coding sequences of the gene or deletion of one or more regulatory elements of the gene.

As an example, the modified seed, cell or plant described herein can be generated using “custom” or engineered endonucleases such as meganucleases produced to modify plant genomes (see e.g., WO 2009/114321; Gao et al. (2010) Plant Journal 1:176-187). Another site-directed engineering is through the use of zinc finger domain recognition coupled with the restriction properties of restriction enzyme. See e.g., Urnov, et al., (2010) Nat Rev Genet. 11(9):636-46; Shukla, et al., (2009) Nature 459 (7245):437-41. A transcription activator-like (TAL) effector-DNA modifying enzyme (TALE or TALEN) is also used to engineer changes in plant genome. See e.g., U.S. 20110145940, Cermak et al., (2011) Nucleic Acids Res. 39(12) and Boch et al., (2009), Science 326(5959): 1509-12. Site-specific modification of plant genomes can also be performed using the bacterial type II CRISPR (clustered regularly interspaced short palindromic repeats)/Cas (CRISPR-associated) system. See e.g., Belhaj et al., (2013), Plant Methods 9: 39; The Cas9/guide RNA-based system allows targeted cleavage of genomic DNA guided by a customizable small noncoding RNA in plants (see e.g., WO 2015026883A1, incorporated herein by reference). These methods are also useful in targeting nucleic acids to pre-engineered target recognition sequences in the genome. A “mutation”, which is possessed by a mutant, refers to a detectable and heritable genetic change (either spontaneous or induced) not caused by segregation or genetic recombination.

Unmodified seed is seed which is similar to the modified seed but which lacks the genetic modification which alters the composition of the seed.

In some embodiments, the methods include measuring a different seed constituent, in combination with measuring a sucrosyl-oligosaccharide to provide additional compositional information, such as a compositional profile, about the seed. Such measurements can be carried out simultaneously with the measurements of sucrosyl-oligosaccharide and may be used to evaluate lines or seeds therefrom, such as modified lines or seeds. A “line” when referring to soybean, is a group of plants of similar parentage that display little or no genetic variation between individuals for a least one trait. Soybean lines are generally homozygous for almost all traits. Lines may be created by one or more generations of self-pollination and selection, or vegetative propagation from a single parent including by tissue or cell culture techniques.

Non-limiting examples of seed constituents which may be measured in the methods provided herein, including processing or sorting seeds, include proteins, oils, carbohydrates, fatty acids (such as one or more of oleic acid, palmitic acid, stearic acid, linoleic acid, linolenic acid, arachidic acid, erucic acid, behenic acid, lignoceric acid and myristic acid) and fatty acid profiles, amino acids, biopolymers, pharmaceuticals, starch, fermentable starch, secondary compounds, sucrosyl-oligosaccharides, metabolites and combinations thereof. For example, alterations, such as increases or decreases, in these constituents can be measured in combination with measuring the amount or concentration of one or more sucrosyl-oligosaccharides in a target or modified soybean seed and compared with a comparable control or unmodified soybean seed which does contain the modification contributing to the altered phenotype.

Soybean seeds that can be used in the methods and systems provided may be transgenic for one or more traits, for example through suppression or over-expression, and/or may have one or more mutations or genetic modifications, that result in a seed having a composition sufficiently different from comparable commodity, non-mutant, non-modified or non-transgenic soybean seeds to enable identification, separation and/or sorting of the transgenic, mutant or genetically modified seed from the comparable or control seed. For example, a low amount or concentration of sucrosyl-oligosaccharide in a soybean seed may indicate that the soybean seed is a modified seed containing one or more traits, such as one or more transgenic events or genetic modifications, that result in a high oil, high protein and/or altered fatty acid profile phenotype in the soybean seed compared with a comparable soybean seed not containing the trait or modification. Soybean seeds that have an amount or concentration of sucrosyl-oligosaccharide below a desired threshold (i.e., below a desired maximal amount) of sucrosyl-oligosaccharide and above a desired threshold (i.e. above a desired minimal amount) of oil content, protein content, or both oil and protein content can be selected and used in plant breeding or industrial processing. The methods described herein can also be used with modified soybean seeds that have an amount or concentration of sucrosyl-oligosaccharide below a desired threshold (i.e., below a desired maximal amount) of sucrosyl-oligosaccharide and above or below a desired threshold for one or more fatty acids. The threshold is selected to enable sorting or separation of modified seeds from the comparable seeds not containing the transgenic trait or genetic modification. Protein meal can also be analyzed rapidly and non-destructively using the methods described herein, and protein meal produced from soybeans containing one or more genetic modifications such as the modifications described herein can be identified from meal produced from non-modified soybeans.

In some embodiments, the genetic modifications include one or more mutations or modifications that result in reduced amounts of stachyose, raffinose or a combination thereof in the soybean seed, such as the low1, low2, low3, low4 mutations described in U.S. Pat. Nos. 5,710,365, 6,147,193 and 6,653,451, mutations in stachyose synthase such as in the PI 603176A and PI 594012 soybean lines (Qui et al., Theor Appl Genet 2015, 128:2167), the mutations in raffinose synthase such as the RS2 or RS3 genes described in U.S. Pat. No. 8,728,726 and U.S. Patent Publication No. 20130318660, the SG-ULRFO mutation described in U.S. Patent Publication No. 20110003045, and the low phytate, low stachyose mutations described in U.S. Pat. No. 8,003,856.

In some embodiments, the genetic modifications include one or more mutations or modifications that result in increased oleic acid, such as in one or more FAD2 alleles, see, e.g. U.S. Pat. Nos. 9,198,365, 9,185,826, 7,531,718, 7,205,457, 7,067,722, 6,426,448, 6,229,033, 5,981,781 and U.S. Patent Publication Nos. 20160186195, 20130219565.

The oleic acid may be increased to about 50%, 55%, 60%, 65%, 70%, 75%, 80%, 85% or 90% (such as at least about or at least 50%, 55%, 60%, 65%, 70%, 75%, 80% or 85% and less than or less than about 95%, 90%, 85%, 80%, 75%, 70%, or 65%; measured as a proportion of the total fatty acids) by suppressing or inhibiting expression of one or more FAD2 genes, such as by mutation, genome editing or transgenes, alone or in combination with other modifications described herein.

In some embodiments, the genetic modifications include one or more mutations or modifications that result in reduced linolenic acid, such as one or more FAD3 or fan (e.g. fan1, fan2, fan3) alleles found, for example, in mutant lines, A5, C1640, RG10, A16, A17, A23, A29 and in soybean lines having such alleles modified by genome editing or transformation. See, e.g. U.S. Pat. Nos. 8,901,375, 7,943,818, 7,205,457, 7,067,722, 6,133,509, 5,850,030, 5,710,369, 5,714,670, 5,763,745, 5,714,668, 5,534,425 and 5,714,670 and U.S. Patent Publication Nos. 20160186195, 20130219565.

The linolenic acid may be decreased to about 6%, 5.5%, 5%, 4.5%, 4%, 3.5%, 3.0%, 2.9%, 2.8%, 2.7%, 2.6%, 2.5%, 2.4%, 2.3%, 2.3%, 2.1%, 2.0%, 1.9%, 1.8%, 1.7%, 1.6%, 1.5%, 1.4%, 1.3%, 1.2%, 1.1% or 1.0% (such as at least about or at least 0.5%, 0.6%. 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.5%, 2.9% or 3.0% and less than or less than about 6%, 5.5%, 5.0%, 4.5%, 4.0%, 3.5%, 3.4%, 3.3%, 3.2%, 3.1%, 3.0%, 2.9%, 2.8%, 2.7%, 2.6%, 2.5%, 2.4%, 2.3%, 2.2%, 2.1%, 2.0%, 1.9%, 1.8%, 1.7%, 1.6%, 1.5%, 1.4%, 1.3%, 1.2%, 1.1% or 1.0% measured as a proportion of the total fatty acids) by suppressing or inhibiting expression of one or more FAD3 genes, such as those disclosed herein and such as by mutation, genome editing or use of transgenes, alone or in combination with other modifications described herein.

In some embodiments, for example, components that can be used to modify the composition of the seed include increased expression of a DGAT (diglyceride acyltransferase; e.g. U.S. Pat. No. 8,153,859; 8,399,736; 9,187,736), such as DGAT1 (e.g. a soy DGAT1 or a modified soy DGAT1 expressing a polypeptide with one or more amino acid substitutions, e.g. U.S. Pat. Nos. 7,524,945, 8,497,362, 8,101,819, 8,455,714; 9,447,386) or DGAT2 (e.g. a Yarrowia lipolytica DGAT2, e.g. U.S. Pat. No. 9,574,207, 8,927,809; 8,993,840), suppression of one or more galactinol synthases (GAS; such as GAS1, GAS2 and GAS3, e.g. U.S. Pat. Nos. 9,574,207; 7,294,756; 6,967,262; 5,648,210; 5,773,699; 5,710,365; 6,147,193; 6,653,451), increased expression of a sucrose transporter, such as SUT2 or SUT4 (e.g. U.S. Pat. No. 8,993,840), expression of transcription factors such as ovule development protein (ODP; also known as Wrinkled1, see, e.g. U.S. Pat. Nos. 8,404,926 and 9,284,571), LEC1 or FUSCA3 (e.g. U.S. Patent Publication No. 20160186195), phosphoglucomutase (PGM; U.S. Pat. No. 8,143,476, 8,829,273), fatty acid desaturase FAD3 (e.g. U.S. Pat. Nos. 7,081,564; 8,609,935; 5,981,781; U.S. Patent Publication No. 20130219565) am iRNA fragments of fad2-1b, fatBF, or fad3c (e.g. U.S. Patent Publication No. 20130219565), carbonic anhydrase (e.g. U.S. Patent Publication No. 20170029836), pectin acetyl esterase (PAE; e.g. U.S. Pat. No. 9,574,204), aldolase such as HpaIL aldolases (e.g. U.S. Pat. No. 9,347,066), cytosolic pyrophosphatase (PPiase; e.g. U.S. Patent Publication No. 20120174261), oxidoreductase and oxidoreductase motifs (ORMs; e.g. U.S. Patent Publication No. 20110219474), or combinations thereof. Promoters that can be used include, for example, one or more of annexin promoter, beta-conglycinin α′-subunit promoter, glycinin 1 promoter, Kunitz trypsin Inhibitor 3 Promoter, albumin 2S promoter, s-adenosylmethionine synthetase promoter, sucrose synthase promoter such as a SUS2 promoter, late embryogensis abundant gene promoter. Other components which can be used include a yeast FLP-Recombinase to facilitate recombination at short flippase recognition target (FRT) sites. Further examples of components are provided in Table 1, which constructs can be used to generate modified or transgenic seeds having a wide range of amounts or concentrations of one or more sucrosyl-oligosaccharides. Any combination if these components described in this paragraph and in Table 1 may be expressed together. One or more of these components may also be combined with one or more of the modifications or mutations described herein, such as mutations or modifications affecting the fatty acid profile such as oleic acid, linolenic acid, linoleic acid, stearic acid or palmitic acid. Sequences disclosed herein include sequences that have at least or at least about 50%, 55%, 60%, 65%, 70%, 75%, 80%, 85%, 90%, 95%, 96%, 97%, 98%, 99% or 100% identity to the disclosed sequences, provided that the sequence functions for its intended purpose.

TABLE 1
List of promoters and gene/amiRNA/RNAi cassettes and their abbreviations

| Feature | | |
|---|---|---|
| Name | Description | Reference |
| ANN | Soy annexin promoter | U.S. Pat. No. 8,084,074 |
| | | (e.g. the sequence |
| | | identified as number 3) |
| BC | Soy beta-conglycinin α′-subunit | Beachy et al., EMBO J. 4, |
| | promoter | 3047-3053 (1985) |
| FAD2 RNAi | RNAi construct containing fragments of | U.S. Pat. No. 7,456,014 |
| | soy fatty acid desaturase 2 genes and | (e.g. the sequence |
| | designed to silence them | identified as number 24) |
| FAD2/ | Combined amiRNA comprising 396b- | U.S. patent application |
| FATBamiRNA | fad2-1b and 159-fatBF | Publication No. |
| | | 20150089689 |
| FAD3amiRNA | amiRNA comprising 159-fad3c | U.S. patent application |
| | | Publication No. |
| | | 20150089689 |
| GAS RNAI | RNAi construct containing fragments of | U.S. Pat. No.7,476,778 |
| | soy galactinol synthases designed to | (e.g. nucleotides encoding |
| | silence them | the sequences identified |
| | | as numbers: 2, 4, 6) |
| | | U.S. Pat. No. 7,456,014 |
| | | (e.g. the sequence |
| | | identified as number 29) |
| GM::HRA/ | Soy Acetolactate Synthase gene and | U.S. Pat. No. 7,456,014 |
| GM-ALS | gene encoding a mutant soy ALS | (e.g. the sequence |
| | enzyme insensitive to sulfonylurea | identified as number 35) |
| | herbicides | U.S. Pat. No. 7,217,858 |
| | | (e.g. the sequences |
| | | identified as numbers 22 |
| | | and 23) |
| GM-DGAT1- | Modified Soy diacylglycerol | U.S. Pat. No. 8, 101,819 |
| C9C10C11 | acyltransferase 1 | |
| GM-ODP1 | Soy Ovule Development Protein 1 | U.S. patent application |
| | | Publication No. 2015- |
| | | 0143583. |
| GM-SUT4 | Soy Sucrose Transporter 4 | U.S. Pat. No. 8,993,840 |
| GY1 | Soybean Glycinin 1 Promoter | U.S. Pat. No. 8,084,074 |
| KTI | Soy Kunitz Trypsin Inhibitor 3 Promoter | Jofuku et al., Plant Cell |
| | | 1: 1079-1093 (1989) |
| LEA | Soy Late Embryogenesis Abundant | U.S. Pat. No. 7,456,014 |
| | gene promoter | |
| PGM RNAi | RNAi construct containing fragments of | U.S. Pat. No. 7,323,560 |
| | soy PGM designed to silence them | |
| SALB | Soy albumin 2S promoter | U.S. Pat. No. 6,177,613 |
| SAMS | Soy S-adenosylmethionine synthetase | U.S. Pat. No. 7,217,858 |
| | promoter | |
| SUS | GM::SUS2 Promoter | U.S. patent application |
| | | Publication No. 2015- |
| | | 0143583 |
| YLDGAT2 | Yarrowia lipolitica diacylglycerol | U.S. Pat. No. 8,143,473 |
| | acyltransferase 2 | U.S. Pat. No. 8, 143,476 |
| FLP- | Yeast FLP-Recombinase | U.S. patent application |
| Recombinase | | Publication No. |
| | | 20160186195 |

Soybeans generated by modifying expression of these sequences and having different amounts of stachyose, raffinose, or both stachyose and raffinose can be used iteratively in the methods described herein to generate calibrations which provide accurate measurements of stachyose or both stachyose and raffinose. In some embodiments verbascose can be measured. As used herein, sucrosyl-oligosaccharide means the sum of stachyose and raffinose.

In some embodiments, the soybean is modified to have suppressed galactinol synthase (GAS) activity with one or more sequences that suppress expression of galactinol synthase (e.g. one or more of GAS1, GAS2 and GAS3) or raffinose synthase (e.g. RS2, RS3) activity (or a combination of GAS and raffinose synthase suppression), alone or in combination with increased DGAT activity (for example, by transforming with a yeast or soy DGAT described in Table 1 or genetically modifying the native DGAT or its regulatory sequences to enhance expression) and optionally other sequences, such as those described in Table 1, to increase oil and/or protein. Such enhanced expression or suppression can be achieved by one or more of genetic modification, such as by gene editing, the use of transgenes, or by mutation. Such seeds may have an amount of stachyose of about 0.1%, 0.2%, 0.3%, 0.4%, or 0.5%, such as at least about 0.05%, or 0.1%, 0.2%, 0.3%, 0.4% or 0.5% and less than about 2%, 1.5%, 1%, 0.9%, 0.8%, 0.7%, 0.6% or 0.5% stachyose (percentage points by weight). Such seeds may have an amount of sucrosyl-oligosaccharide of about 0.3%, 0.4%, 0.5% or 0.6%, such as at least about 0.05%, 0.1%, 0.2%, 0.3%, 0.4% or 0.5% and less than about 2%, 1.5%, 1%, 0.9%, 0.8%, 0.7%, 0.6% or 0.5% sucrosyl-oligosaccharides (percentage points by weight). By contrast, the comparable unmodified, control, null or wild-type seed may have a stachyose content of about 4%, such as at least about 1%, 2%, 2.5%, 3% or 3.5% and less than about 6%, 5.5%, 5% or 4.5% (percentage points by weight). The comparable unmodified, control, null or wild-type seed may have a sucrosyl-oligosaccharide content of about 5%, such as at least about 2%, 2.5%, 3%, 3.5% , 4%, or 4.5% and less than about 6.5%, 6%, 5.5%, or 5% (percentage points by weight).

Such modified seeds may also have an increased oil, protein or combination thereof in addition to reduced sucrosyl-oligosaccharide or stachyose. For example, such modified seeds may have an amount of oil in percentage points by weight of about 23%, 24%, 25%, 26%, 27%, 28%, 29%, 30%, such as at least about 21%, 22%, 23%, 24%, 25%, 26%, 27%, 28%, 29%, 30% and less than about 40%, 35%, 34%, 33%, 32%, 31%, 30%, 29% or 28% oil by weight. In contrast, a comparable unmodified, control, null or wild-type seed may have an amount of oil in percentage points by weight of about 16%, 17%, 18%, 19%, 20%, 21% or 22%, such as at least about 15%, 16%, 17%, 18%, 19%, 20% or 21% and less than about 23%, 22%, 21%, 20%, 19%, or 18% oil by weight. Useful examples of percent point increases in oil or total fatty acid content in a seed, such as a modified soybean seed described herein compared with a comparable or control soybean include, but are not limited to, percentage point increases by weight of at least 1%, 2%, 3%, 4% or 5% and less than 10%, 9%, 8%, 7%, 6%, 5% or 4%. Useful examples of the percent increases in oil or total fatty acid content in a modified soybean seed described herein compared with a comparable unmodified, control, null or wild-type soybean include, but are not limited to, percent increases of at least 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% and less than 99%, 95%, 90%, 80%, 70%, 60%, 50%, 40%, 35%, 30% and 25%.

Such modified seeds may have an amount of protein in percentage points by weight of about 38%, 39%, 40%, 41%, 42%, 43%, 44%, 45%, 46%, 47%, 48%, 49%, 50%, 51%, 52%, 53% or 54% such as at least about 35%, 36%, 37%, 38%, 39%, 40%, 41%, 42%, 43%, 44%, 45%, 46%, 47%, 48%, 49%, or 50% and less than about 55%54%, 53%, 52%, 51%, 50%, 49%, 48%, 47%, 46%, 45%, 44%, 43%, 42%, 41%, 40% or 39% protein. In contrast a comparable unmodified, control, null or wild-type seed may have an amount of protein in percentage points by weight of about 30%, 31%, 32%, 33%, 34%, 35%, 36%, 30 37%, or 38% such as at least about 25%, 26%, 27%, 28%, 29%, 30%, 31%, 32%, 33%, 34%, or 35% and less than about 38%, 37%, 36%, 35%, 34%, 33%, 32% 31%, 30%, 29% or 28% protein. Useful examples of percent point increases in protein (by weight) in a seed, such as a modified soybean seed compared with a comparable or control soybean include, but are not limited to, percentage point increases by weight of at least 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 6%, 7%, 8%, 9%, and 10% and less than 20%, 19%, 18%, 17%, 16%, 15%, 14%, 13%, 12%, 11%, 10%, 9%, 8%, 7%, 6%, 5% or 4%. Useful examples of the percent increases in protein content in a seed such as a modified seed compared with an unmodified, control, null or wild-type soybean seed described herein include, but are not limited to, percent increases of at least 5%, 10%, 15%, 20%, 25%, 30%, 35%, 40%, 45%, 50%, 55%, or 60% by weight and less than about 70%, 65%, 60%, 55%, 50%, 45%, 40%, 35%, 30%, 25%, 20%, 15% or 10%.

The methods can be used to accurately distinguish between individual soybean seeds, or populations or lines of soybeans that differ in percentage points by about 1% stachyose or sucrosyl-oligosaccharide, such as less than 3%, 2.5%, 2%, 1.9%, 1.8%, 1.7%, 1.6%, 1.5%, 1.4%, 1.3%, 1.2%, 1.1%, 1%, 0.9%, 0.8%, 0.7%, 0.6% or 0.5% stachyose or sucrosyl-oligosaccharide by weight and at least 0.1%, 0.2%, 0.3%, 0.4%, 0.5%, 0.6%, 0.7%, 0.85 or 0.9% stachyose or sucrosyl-oligosaccharide by weight. Values are given by weight percent.

The difference in sucrosyl-oligosaccharide, such as stachyose, between seed types being measured, such as a modified and unmodified seed as described herein, can be detected, for example, when the difference is at least 0.5 percentage points, 1.0 percentage points, 1.5 percentage points, 2.0 percentage points, 2.5 percentage points, 3.0 percentage points, or 4.0 percentage points and less than 5.0 percentage points, 4.5 percentage points, 4.0 percentage points, 3.5 percentage points, 3.0 percentage points, 2.5 percentage points, 2.0 percentage points, 1.5 percentage points, or 1.0 percentage points. Values are given by weight percent.

Useful examples of the amount of sucrosyl-oligosaccharide content in a seed such as a modified or unmodified soybean seed include, but are not limited to in percentage points by weight at least or at least about 0.1%, 0.2%, 0.25%, 0.5%, 1%, 1.5%, 2%, 2.5%, 3.0%, 3.5%, 4.0%, 4.5%, 5.0%, 5.5%, 6.0% or 6.5% and less than or less than about 7%, 6.5%, 6.0%, 5.0%, 4.5%, 4.0%, 3.5%, 3.0%, 2.9%, 2.8%, 2.7%, 2.6%, 2.5%, 2.4%, 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3% or 0.2%.

The methods can be used to accurately distinguish between individual soybeans, or populations or lines of soybeans that differ by in percentage points by weight at or about 0.5%, 0.7%, 0.8%, 0.9%, 1.0%, 1.5%, 2.0%, 2.5%, 3%, 3.5%, 4%, 4.5% 5%, 5.5%, 6%, 7%, 8%, 9% or 10% oil, such as in percentage points at least 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 2%. 3%, 4%, or 5% oil and less than 15%, 10%, 5%, 4%, 3%, 2.5%, 2%, 1.9%, 1.8%, 1.7%, 1.6%, 1.5%, 1.4%, 1.3%, 1.2%, 1.1%, 1.0%, 0.9%, 0.8%, 0.7%, 0.6% or 0.5% oil.

The methods can be used to accurately distinguish between individual soybeans, or populations or lines of soybeans that differ in percentage points by weight at or about 1%, 1.5%, 2%, 2.5%, 3%, 3.5% 4%, 4.5%, 5%, 5.5%, 6%, 7%, 8%, 9%,10%, 11%, 12%, 13%, 14%, 15%, 20% or 25% protein, such as at least 0.5%, 1%, 1.5%, 2%, 2.5%, 3%, 3.5%, 4%, 5%, 6%, 7%, 8%, 9%, and 10% protein and less than 30%, 25%, 20%, 15%, 10%, 9%, 8%, 7%, 6%, 5%, 4%, 3%, 2%, or 1% protein.

If a yeast DGAT, such as Yarrowia lipolytica diacylglycerol acyltransferase 2 or a soy DGAT, such as soy DGAT1, is expressed in a soybean seed, optionally with one or more sequences that results in GAS or raffinose synthase suppression, the seed may have an oleic acid content of about 30%, 31% or 32% such as at least about 22%, 23%, 24%, 25%, 26%, 27%, or 28% and less than about 40%, 35%, 34%, or 33%. Unless indicated to the contrary, all percent values for a particular fatty acid are expressed herein as a percentage of the total fatty acid content. The oleic acid can be measured in combination with measuring the amount of sucrosyl-oligosaccharide or stachyose or other components described herein. By contrast, the comparable unmodified, control, null or wild-type seed may have an oleic acid content of about 22% or 23%, such as at least about 19%, 20% or 21% and less than about 25%, 24.5% or 24%. For such modified beans, the threshold for oleic acid to distinguish between the modified and unmodified seed may be about 25% or 30% or 35%, such as at least about 21%, 22%, 23%, 23.5%, 24%, 24.5%, 25%, 25.5%, 26%, 26.5%, 27%, 28%, 29%, 30%, 31%, 32%, 33%, 34%, 35% or 36% and less than about 37%, 36%, 35%, 34%, 33%, 32%, 31%, 30%, 29%, 28%, 27% 27% or 26% oleic acid.

If a soy DGAT, such as the modified soy diacylglycerol acyltransferase 1 described in Table 1, or a yeast DGAT, such as Yarrowia lipolytica diacylglycerol acyltransferase 2 is expressed or over-expressed in a soybean seed, optionally with one or more sequences that results in GAS and/or raffinose synthase suppression, the seed may have a stearic acid content of about 5% (such as at least or at least about 4%, 4.5% or 5% and less than or less than about 10%, 9%, 8%, 7% or 6%) which can be measured in combination with measuring the amount of stachyose or sucrosyl-oligosaccharide or other components described herein. By contrast, the comparable unmodified seed may have a stearic acid content of about 3.5% (such as at least or at least about 3%, 3.1%, 3.2%, 3.3%, 3.4% or 3.5% and less than or less than about 3.7%, 3.8%, 3.9%, 4.0%, 4.1%, 4.2%, 4.3%, 4.4%, 4.5%, 4.6%, 4.7%, 4.8% or 4.9%, 5.0%, 5.5%,).

If a DGAT, such as the modified soy diacylglycerol acyltransferase 1 described in Table 1, is expressed or over expressed in a soybean seed, optionally with one or more sequences that results in GAS suppression, the seed may have a palmitic acid content of about 12% or 13% (such as at least or at least about 11%, 11.1%, 11.2%, 11.3%, 11.4%, 11.5%, 11.6%, 11.7%, 11.8%, 11.9%, or 12% and less than or less than about 15%, 14.5%, 14%, 14.5%, 14%, 13.5%, 13.4%, 13.3%, 13.2%, 13.1%, 13%, 12.9%, 12.8%, 12.7%, 12.6% or 12.5%) which can be measured in combination with measuring the amount of sucrosyl-oligosaccharide or stachyose or other components described herein. By contrast, the comparable unmodified seed may have a palmitic acid content of about 10% or 11% (such as at least or at least about 9%, 9.1%, 9.2%, 9.3%, 9.4%, 9.5%, 9.6%, 9.7%, 9.8%, 9.9% or 10% and less than or less than about 12%, 11.9%, 11.8%, 11.7%, 11.6%, 11.5%, 11.4%, 11.3%, 11.2%, 11.1%, 11%, 10.9%, 10.8%, 10.7%, 10.6%, or 10.5%).

If a DGAT, such as the modified soy diacylglycerol acyltransferase 1 described in Table 1, or such as Yarrowia lipolytica diacylglycerol acyltransferase 2 is expressed or over-expressed in a soybean seed, optionally with one or more sequences that results in GAS suppression, the seed may have a linoleic acid content of about 45% (such as at least or at least about 25%, 30%, 35%, 40% or 45% and less than or less than about 55%, 54%, 53%, 52%, 51%, 50%, 49%, 48%, 47%, 46% or 45%) which can be measured in combination with measuring the amount of sucrosyl-oligosaccharide, such as stachyose or sucrosyl-oligosaccharide or other components described herein. By contrast, the comparable unmodified seed may have a linoleic acid content of about 55% (such as at least or at least about 50%, 51%, 52%, 53%, 54% or 55% and less than or less than about 65%, 64%, 63%, 62%, 61%, 60%, 59%, 58%, 57%, 56% or 55%).

A modified soybean seed such as a soybean seed with enhanced diacylglycerol acyltransferase (DGAT) activity, such as containing a modified soy DGAT1, for example described in Table 1, or a yeast DGAT, such as Yarrowia lipolytica diacylglycerol acyltransferase 2, optionally with one or more modified sequences that results in suppression of one or more GAS sequences, raffinose synthase sequences or both, may have a linolenic acid content of about 5% or 6% (such as at least or at least about 0.1%, 0.5%, 1%, 1.5%, 2%, 2.5%, 3%, 3.5%, 4% 4.5% or 5% and less than or less than about 10%, 9%, 8%, 7.5%, 7%, 6.9%, 6.8%, 6.7%, 6.6%, 6.5%, 6.4%, 6.3%, 6.2%, 6.1% or 6%) which can be measured in combination with measuring the amount of sucrosyl-oligosaccharide or stachyose or other components described herein. A modified soybean seed such as a soybean seed with one or more modified FAD3 genes, such as by mutation, genome editing or transgenes, alone or in combination with one or more of DGAT, GAS, raffinose synthase, and FAD sequences, may have a linolenic content of no more than about 0.5%, 1%, 1.5%, 2%, 2.5% or 3% (such as at least about or at least 0.1%, 0.2%, 0.3%, 0.4%, 0.5%, 0.6%, 0.7%, 0.8%, 0.9%, 1%, 1.5%, 2%, 2.5% or 3% and less than 6%, 5.5%, 5%, 4.5%, 4%, 3.5%, 3%, 2.5%, 2%, 1.5%, 1.4%, 1.3%, 1.2%, 1.1%, 1%, 0.9%, 0.8%, 0.7%, 0.6%, 0.5%, 0.4%, 0.3%, 0.2%, 0.1%, 0.05%). By contrast, the comparable unmodified seed may have a linolenic acid content of about 7% or 8% (such as at least or at least about 5%, 5.5%, 6%, 6.5%, or 7% and less than or less than about 12%, 11%, 10%, 9.5%, 9%, 8.5% or 8%. Linolenic acid may be further reduced in a modified soybean by modifying one or more of the FAD2 alleles such as described herein.

Fingerprints regarding the seed composition can be developed based on the modified light from the soybean seed, wherein a sorting decision is made based upon the measured amounts of sucrosyl-oligosaccharides, in any combination with one or more of the fatty acids (such as oleic acid content or saturated fatty acid content), oil content, or protein content in the soybean seed. The combined measurement can be used to increase accuracy as to whether a seed is modified or not. In some embodiments the soybean seed may contain low sucrosyl-oligosaccharide and, for example, high oil, high protein, one or more altered (increased or decreased) fatty acids, or a combination thereof. In some embodiments, the methods described herein for sorting a seed from a plurality of seeds further includes measuring the amount of oil, one or more fatty acids and/or protein in the seed based on the modified light from the soybean seed, wherein a decrease in the amount of sucrosyl-oligosaccharide correlates with an increase in the oil, protein, altered fatty acid content or combination thereof in the soybean seed.

Threshold values for one or more components can be useful for determining whether a soybean is modified or unmodified. The threshold value for a component measured in a soybean is a value selected to facilitate distinguishing, sorting or separating a soybean, such as a modified soybean, having an amount or concentration of the component that is different (for example, a significantly higher or lower amount or concentration of that component) from another soybean, such as an unmodified soybean.

The threshold value may vary depending on the moisture content of the soybean, and can be set or adjusted to 13% moisture. Values for sucrosyl-oligosaccharides, stachyose, raffinose, oil, protein, total soluble carbohydrate, sucrose and PROIL are provided as percentage points based on weight percent (wt. %). For the fatty acids, such as one or more of oleic acid, palmitic acid, stearic acid, linoleic acid, linolenic acid, values described are expressed as a percentage of that fatty acid relative to the total fatty acid pool.

For sucrosyl-oligosaccharide, (a combination of stachyose and raffinose) the threshold value include values at, about, at least, or at least about 0.1%, 0.2%, 0.25%, 0.5%, 1%, 1.25%, 1.5%, 2%, 2.5%, 3.0%, 3.5%, 4.0% sucrosyl-oligosaccharide and at, about, less than, or less than about 5.0%, 4.5%, 4.0%, 3.9%, 3.8%, 3.7%, 3.6%, 3.5%, 3.4%, 3.3%, 3.2%, 3.1%, 3.0%, 2.9%, 2.8%, 2.7%, 2.6%, 2.5%, 2.4%, 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3% or 0.2% sucrosyl-oligosaccharide.

The threshold for stachyose to distinguish between the modified and unmodified seed may be about 1%, 1.5%, 2%, or 2.5%, such as at least about 0.1%, 0.5%, 0.6%, 0.7%, 0.8%, 0.9%, 1.0%, 1.5%, 2%, 2.5%, 3%, 3.5%, 4%, 4.5% or 5% stachyose and less than about 6.0%, 5.5%, 5%, 4.5%, 4%, 3.5%, 3%, 2.5%, 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3% or 0.2% stachyose.

The threshold for raffinose to distinguish between the modified and unmodified seed may be about 0.7% raffinose such as at least about 0.1%, 0.2%, 0.3%, 0.4%, 0.5%, 0.6%, 0.7%, 0.8%, 0.9%, or 1% raffinose and less than about 1.5%, 1.4%, 1.3%, 1.2%, 1%, 0.9%, 0.8% or 0.7% raffinose.

The threshold value for oil to assist in distinguishing between modified and unmodified seed may be at or about 17%, 18%, 19%, 20%, 21%, 22%, 23%, 24% or 25%, with the modified soybean, such as described herein, containing at least or at least about 19%, 19.5%, 20%, 20.5%, 21%, 21.5%, 22%, 22.5%, 23%, 23.5%, 24%, 24.5%, 25%, 26%, 27%, 28% or 29% oil and less than or less than about 32%, 31%, 30%, 29%, 28%, 27%, 26%, 25%, 24.5%, 24%, 23.5%, 23%, 22.5%, 22%, 21.5%, 21%, 20.5% or 20% oil and the comparable unmodified or null soybean containing at least or at least about 15%, 15.5%, 16%, 16.5%, 17%, 17.5% or 18% oil and less than or less than about 23%, 22.5%, 22%, 21.5%, 21%, 20.5%, 20%, 19.5%, 19%, 18.5%, 18%, 17.5% or 17% oil. The threshold value for oil includes values of at least or at least about 16%, 16.5%, 17%, 17.5%, 18%, 18.5%, 19%, 19.5%, 20%, 20.5%, 21%, 21.5%, 22%, 22.5%, 23%, 23.5% or 24% oil and less than or less than about 26%, 25.5%, 25%, 24.5%, 24%, 23.5%, 23%, 22.5%, 22%, 21.5%, 21%, 20.5%, 20%, 19.5%, 19%, 18.5% or 18% oil.

The threshold value for total protein to distinguish between the modified and unmodified seed may be at or about 32%, 33%, 34%, 35%, 36%, 37%, or 38%, with the modified soybean, such as described herein, containing at least or at least about 37%, 38%, 39%, 40%, 41%, 42%, 43%, 44%, 45%, 46%, 47%, 48%, 49% or 50% protein and less than or less than about 55%, 54%, 53%, 52%, 51%, 49%, 48%, 47%, 46%, 45%, 44%, 43%, 42%, 41% or 40% protein and the comparable unmodified or null soybean containing at least or at least about 30%, 31%, 32%, 33%, 34%, 35%, 36% or 37% protein and less than or less than about 39%, 38%, 37%, 36%, 35%, 34%, 33% or 32% protein. The threshold value for protein includes values of at least 32%, 33%, 34%, 35%, 36%, 37%, 38%, 39%, 40%, 41%, 42%, 43%, 44%, 45%, 46%, 47% or 48% protein and less than or less than about 53%, 52%, 51%, 50%, 49%, 48%, 47%, 46%, 45%, 44%, 43%, 42%, 41%, 40%, 39%, 38%, 37%, 36%, 35%, 34% or 33% protein.

For palmitic acid, a suitable threshold value may be at or about 10.5% or 11%, with the modified soybean such as described herein containing at least or at least about 10.5%, 11%, 12%, or 13% and less than or less than about 20%, 15% 14% or 13% palmitic acid and the comparable unmodified or null soybean containing at least or at least about 5%, 7%, 8%, 9% or 10% and less than or less than about 11%, 10.5%, 10%, 9%, 8% or 7% palmitic acid. The threshold value for palmitic acid includes values at, about, at least or at least about 8%, 9%, 10%, 10.5%, 11% or 12% and at, about, less than or less than about 15%, 14%, 13%, 12%, 11% or 10.5%.

For stearic acid, a suitable threshold value may be at or about 4.5%, with the modified soybean such as described herein containing at least or at least about 4.5%, 5%, 5.5%, or 6% stearic acid and less than or less than about 10%, 9%, 8% or 7% stearic acid and the comparable unmodified or null soybean containing at least or at least about 2%, 2.5%, 3%, 3.5%, or 4% stearic acid and less than or less than about 4.5%, 4%, 3.5%, 3% or 2.5% stearic acid. The threshold value for stearic acid includes values at, about, at least or at least about 3%, 3.5%, 4%, 4.5%, or 5% and at, about, less than or less than about 6%, 5.5%, 5%, 4.5%, 4% or 3.5%.

For oleic acid, a suitable threshold value may be at or about 28%, with the modified soybean such as described herein containing at least or at least about 28%, 30%, 35%, 40%, 45%, 50%, 55%, 60%, 65%, 70%, 75%, 80% or 85% oleic acid and less than or less than about 95%, 90%, 85%, 80%, 75%, 70%, 65%, 60%, 55%, 50%, 45%, 40%, 35% or 30% oleic acid and the comparable unmodified or null soybean containing at least or at least about 10%, 15%, 20%, 25%, or 27% oleic acid and less than or less than about 28%, 25%, 20%, or 15% oleic acid. The threshold value for oleic acid includes values at, about, at least or at least about 18%, 19%, 20%, 21%, 22%, 23%, 24% or 25% and at, about, less than or less than about 75%, 50%, 45%, 40%, 35%, 30% or 28%.

For linoleic acid, a suitable threshold value may be at or about 50%, with the modified soybean such as described herein containing at least or at least about 30%, 35%, 40%, 45%, or 50% linoleic acid and less than or less than about 60%, 55%, 50%, 45%, 40%, or 35% linoleic acid and the comparable unmodified or null soybean containing at least or at least about 50%, 55%, or 60% linoleic acid and less than or less than about 65%, 60%, 55% or 50% linoleic acid. The threshold value for linoleic acid includes values at, about, at least or at least about 45%, 50%, or 55% and at, about, less than or less than about 60%, 55%, or 50%.

For linolenic acid, a suitable threshold value may be at or about 6.5%, with the modified soybean such as described herein containing at least or at least about 0.5%, 1%, 1.5%, 2%, 2.5%, 3%, 3.5%, 4%, 4.5%, 5%, 5.5% or 6% linolenic acid and less than or less than about 6.5%, 6%, 5.5%, 5%, 4.5%, 4%, 3.5%, 3%, 2.5%, 2%, 1.5% or 1% linolenic acid and the comparable unmodified or null soybean containing at least or at least about 6.5%, 7%, 7.5%, 8%, 8.5% or 9% linolenic acid and less than or less than about 10%, 9.5%, 9%, 8.5%, 8%, 7.5%, or 7% linolenic acid. The threshold value for linolenic acid includes values at, about, at least or at least about 1%, 1.5%, 2%, 2.5%, 3%, 3.5%, 4%, 4.5%, 5%, 5.5% or 6% and at, about, less than or less than about 10%, 9.5% , 9%, 8.5%, 8%, 7.5%, 7% or 6.5%.

For total saturated fatty acids (stearic acid plus palmitic acid), a suitable threshold value may be at or about 15.5%, with the modified soybean such as described herein containing at least or at least about 15.5%, 16%, 16.5%, 17%, 17.5%, 18%, 18.5%, 19% or 19.5% total saturated fatty acids and less than or less than about 30%, 25%, 20%, 19%, 18%, 17%, or 16% total saturated fatty acids and the comparable unmodified or null soybean containing at least or at least about 9%, 10%, 10.5%, 11%, 11.5%, 12%, 12.5%, 13%, 13.5%, 14%, 14.5% or 15% total saturated fatty acids and less than or less than about 15.5%, 15%, 14.5%, 14%, 13.5%, 13%, 12.5%, 12%, 11.5%, 11%, 10.5% or 10% total saturated fatty acids. The threshold value for total saturated fatty acids includes values at, about, at least or at least about 10.5%, 11%, 11.5%, 12%, 12.5%, 13%, 13.5%, 14%, 14.5%, or 15% and at, about, less than or less than about 10%, 9.5%, 9%, 8.5%, 8%, 7.5%, 7% or 6.5%.

In some embodiments, soybeans may be modified to have lower total saturated fatty acids (stearic acid plus palmitic acid) than unmodified soybeans, such as at least or at least about 4%, 5%, 6% or 7% total saturated fatty acids and less than about 15%, 12%, 10%, 9%, 8% or 7% with a threshold value of at, about, at least or at least about 5%, 6%, 6.5%, 7%, 7.5%, 8%, 8.5%, 9%, 9.5%, 10%, 10.5%, 11%, 11.5%, 12% or 12.5% and at, about, less than or less than about 18%, 17%, 16%, 15%, 14%, 13%, 12%, 11%, 10%, 9%, or 8%.

For total soluble carbohydrate, a suitable threshold value may be at or about 9%, with the modified soybean such as described herein containing at least or at least about 3%, 3.5%, 4%, 4.5%, 5%, 5.5%, 6%, 6.5%, 7%, 7.5%, 8% or 8.5% total soluble carbohydrate and less than or less than about 6.5%, 6%, 5.5%, 5%, 4.5%, 4%, 3.5%, 3%, 27.5%, 2%, 1.5% or 1% total soluble carbohydrate and the comparable unmodified or null soybean containing at least or at least about 9%, 9.5%, 10%, 10.5%, 11%, 11.5%, 12% or 12.5% total soluble carbohydrate and less than or less than about 15%, 14%, 13%, 12.5%, 12%, 11.5%, 11%, 10.5%, 10% or 9.5% total soluble carbohydrate. The threshold value for total soluble carbohydrate includes values at, about, at least or at least about 4%, 4.5%, 5%, 5.5%, 6%, 6.5%, 7%, 7.5%, 8%, 8.5%, or 9% and at, about, less than or less than about 9.5%, 10%, 10.5%, 11%, 11.5%, 12%, or 12.5%.

For sucrose, a suitable threshold value may be at or about 3.8%, with the modified soybean such as described herein containing at least or at least about 0.5%, 1%, 1.5%, 2%, 2.5%, 3%, or 3.5% sucrose and less than or less than about 3.8%, 3.5%, 3%, 2.5%, 2%, 1.5%, 1%, or 0.5% sucrose and the comparable unmodified or null soybean containing at least or at least about 3.8%, 4%, 4.5%, 5%, 5.5% or 6% sucrose and less than or less than about 7%, 6.5%, 6%, 5.5%, 5%, 4.5%, or 4% sucrose. The threshold value for sucrose includes values at, about, at least or at least about 1%, 1.5%, 2%, 2.5%, 3%, or 3.5% and at, about, less than or less than about 6%, 5.5%, 5%, 4.5%, 4%, 3.5%, 3% or 2.5%.

For the sum of oil and protein content, also referred to as the PROIL content, a suitable threshold value may be at or about 54%, with the modified soybean such as described herein containing at least or at least about 54%, 55%, 56%, 57%, 58%, 59%, 60%, 61%, 62% or 63% PROIL and less than or less than about 70%, 65%, 60%, 59%, 58%, 57%, 56%, or 55% PROIL and the comparable unmodified or null soybean containing at least or at least about 45%, 46%, 47%, 48%, 49%, 50%, 51%, 52% or 53% PROIL and less than or less than about 55%, 54%, 53%, 52%, 51%, 50%, or 49% PROIL. The threshold value for PROIL includes values at, about, at least or at least about 50%, 51%, 52%, 53%, 54%, or 55% and at, about, less than or less than about 64%, 63%, 62%, 61%, 60%, 59%, 58%, 57%, 56%, or 55%.

Useful examples of percent point increases in PROIL in a seed, such as a modified soybean seed compared with a comparable or control soybean include, but are not limited to, percentage point increases of at least or at least about 50%, 55%, 60%, 65%, 70%, 75%, 80%, 85%, 90%, or 95% and less than or less than about 98%, 97%, 96%, 95%, 90%, 85%, 80%, 75%, 70%, 65%, 60%, 65% 60%, 55%, 50%, 45% or 40%.

The methods may be used for seeds from plants with two, three, four, five or ten or more transgenes or genetic modifications, wherein accumulating or stacking of transgenic regions or genetic modifications into plants or lines is achieved by addition of transgenes by transformation, by genome editing, by crossing parent plants or lines containing different transgenic regions or modifications, or any combination thereof. Analyses can be conducted to select individual seeds on the basis of the presence of one or more characteristics associated with at least one transgene or modification. Such characteristics include, but are not limited to, a seed composition, a transgene per se, a genetic marker linked to a transgene or modification, mRNA expressed from a transgene or modification, and a protein product of a transgene or modified region or gene.

Methods and systems provided herein may include the step of confirming a phenotype, for example, by extracting or isolating nucleic acids, such as DNA, from a seed or population of seeds and using appropriate genetic techniques to analyze or detect the genotype. Such genetic techniques include, for example, contacting isolated or extracted nucleic acids with one or more genetic markers, the detection of single nucleotide polymorphisms, simple sequence repeats, restriction fragment length polymorphisms, haplotypes, tag SNPs, alleles of genetic markers, genes, DNA-derived sequences, RNA-derived sequences, promoters, 5′ untranslated regions of genes, 3′ untranslated regions of genes, microRNA, siRNA, quantitative trait loci (QTL), satellite markers, transgenes, mRNA, ds mRNA, transcriptional profiles, and methylation patterns. Examples of genetic analyses to identify or select seeds for trait integration include, without limitation, identification of high recurrent parent allele frequencies, tracking of transgenes of interest or screening for the absence of unwanted transgenes, selection of hybrid testing seed, selection of seed expressing a gene of interest, selection of seed expressing a heritable phenotype, identification of seed with selected genetic loci, and zygosity testing.

Assaying of soybean seeds according to the provided methods and systems can also be done rapidly, with an accurate measure of the composition, such as the amount of sucrosyl-oligosaccharides, of the single soybean or a batch of soybeans achieved in less than 5, 4, 3, 2, or 1 minutes or less than one second following commencement of the method. For example, using FT-NIR, up to 100 g (about 400 to 500) soybean seeds as a single batch can be measured in less than 3, 2 or 1 minutes, for example, in about 1 to 2 minutes or 1 to 3 minutes. For example, using NIT, up to 500 g (about 2,500) soybean seeds can have seed composition, including sucrosyl-oligosaccharides, be measured in less than 3, 2 or 1 minutes, for example, in about 0.5 to 1 minutes, 0.5 to 2 minutes, 0.5 to 3 minutes, or 0.5 to 5 minutes. For example, using SS-NIR, a single seed can be measured in about 1 or 2 minutes, such as 0.5 to 3 minutes, 0.5 to 4 minutes or 0.5 to 5 minutes. Using SS-NIR, a single seed can also be measured in less than a second, such as at least 5, 10, 20, 30, 40, 50, 60, 70, 80, 90 or 100 soybean seeds per second and less than about 1000, 500, 400, 10 300, 200, or 100 seeds per second.

The methods and systems provided herein can enhance efficiency and facilitate high throughput of sorting and selecting seed and plants grown from the seed with a desired trait.

In some embodiments, the methods described herein are used in transgenic, genome modification or research breeding programs where sample size may be limited, such as a single seed from a segregating plant, and when intact viable seed are required for propagation and advancement. In some embodiments, the methods are used for seed analysis where destructive analytical methods are not desirable because intact seeds are required for processing or when there is insufficient time to undertake destructive analysis.

The progeny seed can be selected, bulked and used to make further breeding crosses or in further research. The progeny seed can be made subject to the methods of non-destructive analysis provided herein.

Also provided herein is a method for producing a soybean plant with one or more desired traits, e.g. transgenes or modifications. Donor soybean plants for a parental line containing the desired trait are selected. Selected plant material may represent, among others, an inbred line, a hybrid line, a heterogeneous population of soybean plants, or an individual plant. According to techniques well known in the art of plant breeding, the donor parental line is crossed with a second parental line. In some embodiments, the second parental line is a high yielding line. This cross produces a segregating plant population composed of genetically heterogeneous plants. Seeds of plants of the segregating plant population are screened for the desired trait using the analytical methods as disclosed herein. Further breeding may include, among other techniques, additional crosses with other lines, hybrids, backcrossing, or self-crossing. The result is a line of soybean plants that has the desirable trait and optionally also has other desirable traits from one or more other soybean lines.

The methods and systems provided herein provide an increased capacity to evaluate a larger number of breeding populations per field unit, and increased capacity to analyze breeding populations for desired traits prior to planting. For example, the methods and systems allow a breeder to analyze at least 100, 250, 500, or 1,000 seeds and sort or select the 5, 10, 25 or 50 desired seeds from that population for planting without having to plant assess, tag and sample the original population of 100, 250, 500, or 1,000 seeds. Very large sample sizes can be processed quickly by either single seed or bulk analysis, such as at least about 1 kg, 5 kg, 10 kg, 100 kg, 500 kg, 1000 kg, 1500 kg, 2000 kg, 3000 kg, 4000 kg or 5000 kg of soybean seeds per hour and less than 25,000 kg, 20,000 kg, 15,000 kg, 10,000 kg, 7,500 kg, 5,000 kg, 2,000 kg, 1000 kg, 500 kg, 100 kg, 10 kg or 5 kg soybean seeds per hour.

The methods and systems provided herein further permit quality assurance (QA) and quality control (QC) by assuring that soybean seeds are free of regulated or unwanted transgenes, undesirable genetic traits, or undesirable inherited phenotypes by identifying such phenotypes and discarding such seed.

Soybean seeds which can be used may additionally contain desirable agronomic traits that enhance production and consistency of production of soybean grain, such as herbicide tolerance, disease resistance, insect resistance, increased grain yield, increased nutritional content, increased growth rates, enhanced stress tolerance, altered maturity, and combinations thereof. Quality traits such as higher oil, higher protein, modifications in essential amino acids and protein compositional changes, changes in oil composition, nutritional traits such as vitamins, and traits with industrial uses including biodiesel, biolubricants, and polymers can also be identified and selected.

The methods and systems can be used in a plant breeding program which selects plants or seeds having a desired genetic or phenotypic trait, wherein a desired genetic trait includes one or more of a genotype, a haplotype, an allele, a sequence, a transcript profile, and a methylation pattern. The methods and systems may be further used in combination with plant breeding methods where a single seed selected or sorted is crossed or backcrossed and a single generation or multiple generations of progeny plants are generated. Seed of the progeny plants may be processed according to the methods described herein. The crossing and backcrossing steps of the first and subsequent generation of progeny plants and seeds may be carried out in any combination. The choice of breeding method depends on the mode of plant reproduction, the heritability of the trait(s) being improved, and the type of cultivar used commercially (e.g., hybrid cultivar, pureline cultivar, etc.). Selected, non-limiting approaches for breeding the plants are set forth below. It is further understood that any soybean lines, varieties or cultivars can be utilized in a breeding program. Factors including, for example, without limitation, emergence vigor, vegetative vigor, stress tolerance, disease resistance, branching, flowering, seed set, seed size, seed density, standability, and threshability may be selected for use in the breeding program.

In some embodiments, the single seed identity of the seed is preserved. Several methods of preserving single seed identity can be used while transferring or transporting seed from the location of the seed, such as at or following harvest, to the location where analysis is conducted, to the field or greenhouse location where the selected plants are to be grown. Methods include, but are not limited to, transferring selected individuals to seed tape, a cassette tray, or indexing tray, transplanting with peat pots, and hand-planting from individual seed packets.

The apparatus, device, system or method for measuring and sorting seeds can comprise or use a transport system which supports at least one seed at a time and exposes the at least one seed to an optical interrogation device or an imaging system, such as NIT, NIR or FT-NIR, which captures at least one near-infrared image of the at least one seed. The imaging system can be configured to accurately measure the amount of one or more sucrosyl-oligosaccharides in the first seed compared with the standard reference analytical method provided herein. The apparatus, device, system or method can include an electronic controller which makes a sorting decision with at least two sorting outcomes regarding the seed based on the modified light, image or near infrared spectra obtained from the seed and a sorting system to alter the path of the seed based on the sorting decision, wherein the electronic controller associates a first sorting outcome with the first seed and the electronic controller associates a second sorting outcome with the second seed. Seed containing low amounts of sucrosyl-oligosaccharides below (or at) a threshold value can be separated from seed containing higher amounts of sucrosyl-oligosaccharides above (or at) the threshold value. For example, greater confidence in the sorting decisions may achieved by combining the sucrosyl-oligosaccharide threshold value with a threshold value for one or more fatty acid percentages, altered, such as increased oil or protein content, changes in the soluble sugar levels, or a combination thereof. Each of these parameters can be measured from the same near infrared spectra captured from a single seed, bulk seeds or protein meal.

In some embodiments, a method for determining the amount of a sucrosyl-oligosaccharide in a single soybean seed or a sample of intact soybean seeds, comprises directing light from a light source onto a soybean seed or seeds to form modified light from the soybean seed or seeds; receiving the modified light in an imaging device, such as capturing the near infrared absorption spectra, and measuring the amount of a sucrosyl-oligosaccharide in the seed or seeds based on the received modified light, the amount of sucrosyl-oligosaccharide being measured to an accuracy according to the standard reference analytical method provided herein.

The imaging device can be a commercially available infrared spectrometer, including for example, an infrared spectrometer, a Fourier transform infrared spectrometer, or a spectrophotometer with a diffuser and lens and filter array such as described in U.S. Pat. Nos. 9,500,523, 9,383,258, 9,377,396 and 9,291,504, or a seed sorting device such as described in U.S. Pat. No. 8,907,241 or a device useful for single seed analysis such as described in U.S. Pat. No. 8,965,060.

In some embodiments, a method for processing seeds or for determining the amount of a sucrosyl-oligosaccharide in a soybean seed comprises directing light from a light source onto an individual soybean seed to form modified light from the soybean seed; receiving the modified light in an imaging device; measuring the amount of a sucrosyl-oligosaccharide in the seed based on the received modified light, the amount of sucrosyl-oligosaccharide being measured to an accuracy that is within the parameters provided herein. The seed can be transported to a first location when the amount of sucrosyl-oligosaccharide measured is below a threshold value and transporting the seed to a different second location when the amount of sucrosyl-oligosaccharide measured is at or above the threshold value. The seed can be separated into modified and unmodified seed wherein the amount of sucrosyl-oligosaccharide, such as stachyose or a combination of stachyose and raffinose, differ between the modified and unmodified seed as described herein.

In some embodiments, the method or system includes an automated method or system, wherein a seed is separated from a plurality of seeds prior to directing light from a light source onto an individual soybean seed to form modified light from the soybean seed. The light source can be comprised in an optical interrogation device or system and comprises near infrared light such as a broad spectrum light source or a near infrared light source. The automated system may include a transport system for transporting separated seeds to the optical interrogation device and for transporting the seeds to one, two, three or more different locations based on the composition of the seed measured by the optical interrogation device. Individual seeds can be automatically transported to a first station for receiving light where light is directed from a light source on the individual soybean seed at a first station to form modified light, the modified light from the soybean seed is received in an imaging device, and the amount of one or more sucrosyl-oligosaccharides in the seed measured based on the modified light, to an accuracy as provided herein. The method may further comprise following the measurement of the amount of a sucrosyl-oligosaccharide, transporting the seed to a first location when the amount of sucrosyl-oligosaccharide measured is below a threshold value and transporting the seed to a different second location when the amount of sucrosyl-oligosaccharide measured is at or above the threshold value. All of the prior steps can be repeated for a second and subsequent individual seeds.

In some embodiments, a plurality of seeds to be measured either as single seeds or as a sample of intact seeds includes both modified and unmodified seeds as described herein, wherein the modified seeds are transported to the first location and the unmodified seeds are transported to the second location based on the compositional differences detected by the optical interrogation device. The steps of sorting a seed from a plurality of seeds and carrying out this method as disclosed herein can be repeated for at least a second, third or fourth seed. In some embodiments, seeds are selected or separated depending on the composition measured using the methods described herein.

The methods are suitable for measuring and processing small and large sample sizes. In some embodiments, a sample or population of at least 5, 10, 15, 20, 25, 30, 40, or 50 seeds and less than 250, 225, 200, 175, 150, 125, 100, 90, 80, 70, or 60 whole, intact and/or viable seeds are assayed together as a batch of seeds in the methods and systems provided. In some embodiments, a sample of at least 1 g, 5 g, 10 g, 100 g, 150 g, 200 g, 250 g, or 300 g seeds and less than 5000 g, 2000 g, 1000 g, 900 g, 800 g, 700 g, 600 g, or 500 g whole, intact and/or viable seeds are assayed together as a batch of seeds in the methods and systems provided. In some embodiments, at least 5, 10, 15, 20, 25, 30, 40, 50, 75, 100, 150, 200, 250, 500 or 1000 and less than 1,000,000, 500,000,100,000, 75,000, 50,000, 25,000, 10,000, 5,000, 2,500, 1,000, 500, 250, 100, 75, 50 or 25 individual seeds, seed, meal or soybean flake batches, or seed populations are measured.

Methods and systems provided can be used to analyze individual seeds or seed batch samples within a population of seeds to measure one or more compositional differences in one or more components of the seed or seeds.

Following the measurement of the composition of the seeds, for example, the amount of sucrosyl-oligosaccharide, oil, protein, sugars, starch, carbohydrate, fiber or combination thereof, the soybean seeds can be processed. Processing steps can include one or more of dehulling of the soybeans, extraction of oil, for example by use of solvents, processing soy flakes to soy meal for animal feed, grinding soy flakes to produce soy flour, sizing soy flakes to produce soy grits or texturizing soy flakes to produce textured vegetable protein. Soy protein concentrates and isolated soy protein can be further refined and produced from soy flakes. The methods and systems provided may include the step of processing the soybeans into meal without the need for dehulling, based on the low non-digestible carbohydrate content, including one or more of stachyose and raffinose. The composition of the soy beans can be accurately measured in the field or at the grain elevator to facilitate processing decisions on a large scale. Protein meal can be accurately measured at the grain processing plants to determine meal quality and value.

Some embodiments include methods and systems for selecting a plant or plant seed, comprising directing light from a light source onto an individual soybean seed to form modified light from the soybean seed; receiving the modified light in an imaging device; measuring the amount of a sucrosyl-oligosaccharide in the seed based on the received modified light, the amount of sucrosyl-oligosaccharide being measured to an accuracy that is within an amount measured using the standard reference analytical methods as provided herein; transporting the seed to a first location when the amount of sucrosyl-oligosaccharide measured is below a threshold value and transporting the seed to a different second location when the amount of sucrosyl-oligosaccharide measured is at or above the threshold value. The seed from the first location and/or the second location can be grown to produce a plant which can be crossed with a different plant or selfed/allowed to self. The progeny seed produced from seed at the first location may contain a lower or reduced amount or concentration of sucrosyl-oligosaccharide, when compared to progeny seed produced from seed at the second location. The seed transported to either the first or second location and produced through crossing or selfing can be transgenic or non-transgenic and may comprise at least one recombinant construct in the genome, or may not comprise a recombinant construct in the genome. The seeds selected by the methods as disclosed herein may be further selected and used in breeding.

Some embodiments include methods for sorting seeds, the methods comprising directing light from a light source onto an individual soybean seed to form modified light from the soybean seed receiving the modified light in an imaging device measuring the amount of a sucrosyl-oligosaccharide in the seed based on the received modified light, the amount of sucrosyl-oligosaccharide being measured to an accuracy that is within 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 percentage points of the amount measured using the standard reference analytical method; and transporting the seed to a first location when the amount of sucrosyl-oligosaccharide measured is below a threshold value and transporting the seed to a different second location when the amount of sucrosyl-oligosaccharide measured is at or above the threshold value. The method of sorting as disclosed herein may further comprise growing a plant from the sorted seed and crossing the plant with itself or a different plant and using the seed and plant in a breeding program as provided herein.

Any seed, including monocot and dicot seeds, may be adapted to be utilized in a method, systems or device provided herein. The seed may be, for example, alfalfa seed, apple seed, banana seed, barley seed, bean seed, broccoli seed, castor bean seed, citrus seed, clover seed, coconut seed, coffee seed, maize seed, cotton seed, cucumber seed, Douglas fir seed, Eucalyptus seed, Loblolly pine seed, linseed seed, melon seed, oat seed, olive seed, palm seed, pea seed, peanut seed, pepper seed, poplar seed, Radiata pine seed, rapeseed seed, rice seed, rye seed, sorghum seed, Southern pine seed, soybean seed, strawberry seed, sugar beet seed, sugarcane seed, sunflower seed, sweetgum seed, tea seed, tobacco seed, tomato seed, turf seed, wheat seed, and Arabidopsis seed.

Depending on the seed composition, the seed may be discarded prior to planting or planted such as when being used in a plant breeding program, or can be directed to an appropriate processing plant or process if the seed is harvested from a crop and intended for processing. Processing that may occur, depending on the seed composition, may include or exclude one or more of the steps of dehulling, extracting oil, processing meal and producing protein from the soybean. In general, soybean oil is produced from cleaned, tempered, dehulled, and flaked soybeans using solvent (hexane) extraction or a combination of physical pressure and/or solvent extraction.

The following examples are presented in order to more fully illustrate some embodiments of the invention. They should, in no way be construed, however, as limiting the broad scope of the invention. Those of ordinary skill in the art will readily adopt the underlying principles of this discovery to design various compounds without departing from the spirit of the current invention.

### EXAMPLES

In the following Examples, parts and percentages are by weight and degrees are Celsius, unless otherwise stated. The meaning of abbreviations is as follows: “sec” means second(s), “min” means minute(s), “h” means hour(s), “d” means day(s), “μL” means microliter(s), “mL” means milliliter(s), “L” means liter(s), “μM” means micromolar, “mM” means millimolar, “M” means molar, “mmol” means millimole(s), “μmole” mean micromole(s), “g” means gram(s), “μg” means microgram(s), “ng” means nanogram(s), “U” means unit(s), “bp” means base pair(s) and “kB” means kilobase(s).

### Example 1

Creation of Events Having Compositional Diversity

Soybean lines having a broad range of compositional diversity were created as described below.

RNAi was used to silence seed-specific gene expression of soy fatty acid desaturase 2 (fad2) to produce soybeans with a seed oil composition having increased oleic acid according to U.S. Pat. No. 7,456,014.

RNAi was used to silence seed-specific gene expression of soy phosphoglucomutase (PGM) to produce soybeans with a seed oil composition having increased oil and protein according to U.S. Pat. No. 7,323,560.

RNAi comprising polynucleotide fragments were prepared for galactinol synthase 1 (GAS1), according to U.S. Pat. No. 5,648,210, galactinol synthase 2 (GAS2) according to U.S. Pat. No. 6,967,262 and galactinol synthase 3 (GAS3) according to U.S. Pat. No. 7,294,756. The use of seed-specific silencing of gene expression of soy galactinol synthases (GAS) to produce soybeans with a decreased raffinose and stachyose carbohydrate content and increased sucrose content was carried out according to PCT/US14/48825.

Artificial microRNAs (am iRNAs) were used to silence seed-specific gene expression of soy fatty acid desaturase 3 (fad3) to produce soybeans with a seed oil composition having decreased alpha-linolenic acid according to PCT/US13/22654 and PCT/US14/48825.

Artificial microRNAs (amiRNAs) were used to silence seed-specific gene expression of soy fatty acid desaturase 2 (fad2) to produce soybeans with a seed oil composition having increased oleic acid according to PCT/US08/87082, PCT/US13/22654 and PCT/US14/48825.

Artificial microRNAs (amiRNAs) were used to silence seed-specific gene expression of soy fatty acid thioesterase 2 (fatB) to produce soybeans with a seed oil composition having decreased palmitic and stearic acids according to, for example, PCT/US13/22654 and PCT/US14/48825. Combining amiRNAs together to silence multiple genes such as fad2 and fatB was carried out according to, for example, PCT/US13/22654 and PCT/US14/48825.

A modified soy diacylglycerol acyltransferase 1 gene (GM-DGAT1-C9C10C11) under control of a seed-specific promoter was used to produce soybeans having higher oil and protein in the seed according to, for example, U.S. Pat. No. 8,101,819 and PCT/US14/48825.

A Yarrowia lipolytica diacylglycerol acyltransferase 2 gene (YL-DGAT2) under control of a seed-specific promoter was used to produce soybeans having higher oil and protein in the seed according to U.S. Pat. Nos. 8,143,473, 8,143,476 and in PCT/US14/48825.

YL-DGAT2, under control of a seed-specific promoter, was combined with a fad3 amiRNA under control of a seed-specific promoter, and with a GAS RNAi cassette under control of a seed-specific promoter according to PCT/US14/48825.

A soy sucrose transporter 4 (GM-SUT4) alone or in combination with YL-DGAT2 under control of a seed-specific promoter was used to produce soybeans having higher seed oil has according to U.S. Pat. No. 8,993,840.

A soy ovule development protein 1 (GM-ODP1) alone or in combination with YL-DGAT2 or GM-DGAT1-C9C10C11 under control of the soy sucrose synthase promoter was used to produce soybeans having higher oil and protein in the seed according to PCT/US12/70828.

Mutations in the sucrosyl-oligosaccharide pathway, such as low2 (mutant with reduced raffinose synthase expression leading to low stachyose and raffinose and elevated sucrose and galactinol and low4 (mutant with reduced myo-inositol-1P-synthase leading to low stachyose and raffinose) according to U.S. Pat. No. 6,653,451.

Wildtype commodity soybeans were also included in the test set along with transgenic null materials (i.e., lines that had undergone the typical transformation process but that were found, on subsequent analysis, to not be expressing the trait of interest). Varieties used included one or more of the following commercial or public varieties: 91M10, 92Y51, 92Y61, 93E367, 93E368, 93E386, 93M02, 93M11, 93M12, 93Y21, 93Y30, 93Y41, 93Y42, 93Y83, 93Y84, 94Y23, 95E334, 98Y11, ASGA232HS, EX82J07, JACK, P29T68PR, P32T8OPR, SP6634911, YR25C09, YR37Y09.

A list of experiment names and the corresponding DNA constructs used to create soybean events having a range of compositions as described herein is shown in Table 2.

TABLE 2
Experiment names and corresponding DNA plasmids/DNA fragments used
to create soybean events producing a diverse range of compositions.

| | | DNA | | Second DNA | |
|---|---|---|---|---|---|
| Experiment | Transformation | Plasmid/ | SEQ | Plasmid/ | SEQ |
| Name | Type | Fragment | ID NO: | Fragment | ID NO: |
| Soil2 | SSI | PHP48070 | 1 | n/a | n/a |
| Soil19 | SSI | PHP50573 | 2 | n/a | n/a |
| Soil91 | SSI | PHP64612 | 3 | n/a | n/a |
| Soil92 | SSI | PHP64613 | 4 | n/a | n/a |
| Meal 18 | Random Particle | PHP25066A | 5 | n/a | n/a |
| | Bombardment | | | | |
| Oil119 | Random Particle | PHP64207A | 6 | n/a | n/a |
| | Bombardment | | | | |
| HOGAS | Random Particle | PHP17522A | 7 | PHP17734A | 8 |
| | Co-Bombardment | | | | |
| Meal34 | Random Particle | PHP29252A | 9 | PHP19031A | 10 |
| | Co-Bombardment | | | | |
| Meal36 | Random Particle | PHP29882A | 11 | PHP29959A | 12 |
| | Co-Bombardment | | | | |

The Soil 2 PHP48070 plasmid contains the following noted sequences: GAS hairpin from position: 13833-17206 of SEQ ID NO: 1, FAD2-specific amiRNA precursor from position: 6980-8557 of SEQ ID NO: 1, and the FAD3-specific amiRNA precursor from position: 10514-11472 of SEQ ID NO: 1.

The Soil 19 PHP50573 plasm id contains the following noted sequences: GAS hairpin from position: 13809-17182 of SEQ ID NO: 2, FAD3-specific amiRNA precursor from position: 10490-11448 of SEQ ID NO: 2, and the YL-DGAT2 from position: 6996-8540 of SEQ ID NO: 2.

The Soil 91 PHP64612 plasm id contains the following noted sequences: GAS hairpin from position: 15112-18485 of SEQ ID NO: 3, Gm_SUT4 from position: 12428-13945 of SEQ ID NO: 3, Gm_DGAT1 from position: 9879-11390 of SEQ ID NO: 3, and Gm_ODP1: from position: 6823-8058 of SEQ ID NO: 3.

The Soil 92 PHP64613 plasm id contains the following noted sequences: GAS hairpin from position: 17136-18849 of SEQ ID NO: 4, Gm_SUT4 from position: 12961-14478 of SEQ ID NO: 4, Gm_DGAT1 from position: 9454-10998 of SEQ ID NO: 4, and Gm_ODP1: from position: 6823-8058 of SEQ ID NO: 4.

The Meal 18 PHP25066A plasmid contains the following noted sequences: GAS suppression fragments from position: 76-2194 of SEQ ID NO: 5.

The Oil 119 PHP64207A plasmid contains the following noted sequences: GAS hairpin from position: 10496-13869 of SEQ ID NO: 6, Gm_SUT4 from position: 7812-9329 of SEQ ID NO: 6, and Gm_DGAT1 from position: 5262-6773 of SEQ ID NO: 6.

The HOGAS PHP17734A plasmid contains the following noted sequences: GAS suppression fragments from position: 1132-1977 of SEQ ID NO: 8 and a FAD2 suppression fragment from position: 5376-5986 of SEQ ID NO: 8. The HOGAS plasmid PHP17522A (SEQ ID NO: 7) contains a selectable marker (herbicide resistance).

The Meal 34 PHP29252A plasm id contains the following noted sequence: GAS/PGM hairpin structure from position: 2117-6630 of SEQ ID NO: 9. The Meal 34 plasmid PHP19031A (SEQ ID NO: 10) contains a selectable marker (herbicide resistance).

The Meal 36 PHP29882A plasmid contains the following noted sequence: PGM hairpin from position: 634-1973 of SEQ ID NO: 11. The Meal 36 PHP29959A plasmid contains the following noted sequence: GAS hairpin from position: 321-3694 of SEQ ID NO: 12.

Ascl fragments were prepared transformed into soy using particle gun bombardment, events were selected, plants grown and seed were harvested as described in U.S. Pat. No. 8,084,074 for random particle bombardment [Meal18 (PHP25066A) or Oil119 (PHP64207A)] and random particle co-bombardment [HOGAS (PHP17522A+PHP17734A), Meal34 (PHP29252A+PHP19031A) or Meal36 (PHP29882A +PHP29959A)] experiments.

Transgenic SSI target event “A”, previously described in U.S. Pat. No. 8,293,533, was transformed with the donor constructs [Soil2 (PHP48070), Soil19 (PHP50573), Soil91 (PHP64612) or Soil92 (PHP64613)] and the FLP recombinase construct PHP44664 as previously described in PCT/US14/48825 and events were selected, plants grown and seed were harvested as described previously and in U.S. Pat. No. 8,084,074.

### Example 2

Reference Chemistry for Development of Single Seed Compositional Models.

2.1. Sample Preparation for Single Seed Reference Chemistry.

A single soybean was placed in a Spex Certiprep ½×2″ polycarbonate vial with cap (cat #3116PC). A ⅜″ stainless steel ball bearing was added. Grinding was performed in a Spex Certiprep 2000 Geno/Grinder at 1500 strokes/min for three 30 second intervals with a 1-minute rest between each cycle.

2.2. Lipid and Non-Structural Carbohydrate Extraction; GC Fatty Acid Profile Determinations

Quantitative oil determinations were performed (on both the whole and ground bean samples) by NMR (see below). The lipid extracts of the single soybean powders were used solely to determine the fatty acid profiles. Three replicate extractions were performed on each sample as follows:
2.2.1. Weigh sample (approximately 20-50 mg; to an accuracy of 0.1mg) into 13×100 mm tube (with Teflon® lined cap; VWR (53283-800) and record weight. In later studies sample size was standardized at ˜20.0 mg.
2.2.2. Add 2 mL Heptane, vortex and place into an ultrasonic bath (VWR Scientific Model 750D) at 60° C. for 15 min at full sonification-power (˜360W).
2.2.3. Centrifuge for 5 min at 1700×g at room temperature.
2.2.4. Decant the supernatant to a clean 13×100 mm glass tube.

2.3. Fatty Acid Profile Determination: GC Method:
2.3.1. Transfer 200 uL aliquot of the heptane extract into a clean screw top GC vial National Scientific (C4000-186W)
2.3.2. To the 200 uL add 300 uL heptane and 50 uL trimethylsulfonium hydroxide in methanol (JenaChem)
2.3.3. Shake the vials on an orbital shaker at room temperature for 15 minutes.
2.3.4. The fatty acid methyl esters were analyzed by directly injecting 1 uL samples (at a 5:1 split ratio) onto an Agilent 6890 gas chromatography system fitted with a Supelco Omegawax 320 (30 m×0.320 mm×0.25 um film) capillary column. Hydrogen was used as the carrier gas (39 cm/sec average linear velocity). Inlet and FID detector temperatures were held at 260° C. and the oven column temperature was ramped from 180 to 240° C. at a rate of 12° C. per minute.

2.4. Non-structural Carbohydrate Extraction:
2.4.1. Add 1 mL acetone to the heptane extracted pellet from the fatty acid profile method above, vortex mix to disperse the material into the acetone and dry in a SpeedVac.
2.4.2. To the dry pellet add 2 mL of 80% ethanol. Vortex to break up pellet as much as possible. Extract on sonicator (see 2.2.2) for 15 min at 60° C.
2.4.3. Centrifuge for 5 min at 1700×g. Transfer supernatant to a clean 13×100 mm tube.
2.4.4. Repeat Steps 2.4.2 and 2.4.3 two more times, combining the supernatant with the above (3) each time.
2.4.5. Add 100 μL of phenyl-β-D glucopyranoside internal standard (β-phenyglucopyranoside stock 0.5000+/−0.0010 g in 100 ml water) to the combined supernatant. Dry the extract in a SpeedVac and analyze for non-structural carbohydrates as described below.
2.4.6. Add 1 ml acetone and dry the remaining pellet in the SpeedVac.

2.5. Starch Digestion and Extraction:
2.5.1. Perform starch digestion directly on the acetone dried pellets from non-structural carbohydrate extraction.
2.5.2. Add 100 units of a-Amylase (a-amylase; Heat Stable from Bacillus licheniformis e.g. Sigma-Aldrich A-4551) in 0.9 mL 50 mM MOPS (3-(N-Morpholino) propane sulfonic acid) buffer pH 7.0, containing 5 mM CaCl₂ and mix.
2.5.3. Place tubes into a heating block at 90° C. for 75 minutes. Mix several times during hydrolysis.
2.5.4. Allow tubes to cool to room temperature and add 5 units of Amyloglucosidase (commercially available from Roche 11 202 367 001) in 0.6 mL of 285 mM acetate buffer, pH 4.5 and incubate in a reciprocating water bath at 55° C. for 15-18 hours.
2.5.5. Remove rack of tubes and bring to room temperature.
2.5.6. Add 4.5 mL of absolute ethanol to each tube to attain a final ethanol concentration 80% and vortex. Extract on sonicator for 15 min at 60° C.
2.5.7. Centrifuge 5 min at 1700×g and decant supernatant to a 13×100mm tube and immediately place tube in SpeedVac to reduce the volume. 2.5.8. Extract pellet a further 2 times with 2 mL 80% ethanol, combining supernatant with above each time. 2.5.9. Add 100 μL of phenyl-β-D glucopyranoside (see 2.4.5) to the combined supernatant before it is fully dry. Once the extract in the SpeedVac is dry analyze for non-structural sugars as described below. 2.5.10. Add 1 ml acetone and dry the remaining pellet in the SpeedVac and store (at −20° C.) for structural sugar analysis.

2.6. Total soluble Carbohydrate Derivatization and Analysis.
2.6.1. The dried samples from the soluble and starch extractions described above along with sets of sugar standard (pinitol, sorbitol, fructose, glucose, β-phenyl glucopyranoside, sucrose, raffinose and stachyose; at 0, 0.05, 0.10, 0.50, 1.00, 2.00, 3.00, 4.00 and 5.00 mg/tube) mixtures were solubilized in anhydrous pyridine (Sigma-Aldrich P57506) containing 30 mg/ml of hydroxylamine HCl (Sigma-Aldrich 159417).
2.6.2. Samples were placed on an orbital shaker (300 rpm) overnight and were then heated for 1 hr (75° C.) with vigorous vortex mixing applied every 15 min.
2.6.3. After cooling to room temperature 1 ml hexamethyldisilazane (Sigma-Aldrich H-4875) and 100 μL trifluoroacetic acid (Sigma-Aldrich T-6508) were added. The samples were vortex mixed and the precipitates were allowed to settle prior to transferring the supernatants to GC sample vials.
2.6.4. Samples were analyzed on an Agilent 6890 gas chromatography system fitted with a DB-17MS capillary column (30 m×0.32 mm×0.25 um film). Inlet and detector temperatures were both 275° C. After injection (2 μL, 20:1 split) the initial column temperature (150° C.) was increased to 180° C. at a rate 3° C./min and then at 25° C./min to a final temperature of 320° C. The final temperature was maintained for 10 min. The carrier gas was H₂ at a linear velocity of 51 cm/sec. Detection was by flame ionization. A lm length of plain 0.320 mm capillary tube (Agilent; 160-2325-5) was inserted between the inlet and the analytical column to act as a guard column. The two column sections were connected using a push-fit connector. Prior to all analytical runs three injections of a standard mixture containing 5 mg of each sugar was made to passivate the chromatography system. This process was found to enable full recovery of stachyose from the analytical samples, especially as the column aged. Ultra-Inert Inlet Liners (Agilent; 5190-3164) were also used and were routinely changed based on indications of loss in chromatographic performance.
2.6.5. Data analysis was performed using Agilent ChemStation software. Each sugar was quantified relative to its own calibration curve, after dividing each individual peak by the area of the internal standard in each sample and standard. Final carbohydrate concentrations were expressed on a weight percent basis, corrected for moisture content as set forth herein. Residual sucrose, raffinose and stachyose recovered in the starch digestions were included in the total values reported for each sugar.

2.7. Supplemental Methods.

Moisture Content Determinations were Performed according to American Oil Chemists Society (AOCS Official Method Ba 2a-38, Modified for Small Samples) as follows:
2.7.1. Weigh powdered sample material (approximately 100 mg; to an accuracy of 0.1 mg) into a pre-weighed (and recorded) 13×100 mm glass tube VWR (53283-800) and weigh again.
2.7.2. Place samples into a forced air oven preheated to 130° C.
5 2.7.3. Allow material to dry for 2 h.
2.7.4. Remove tubes into a desiccator cabinet and allow to come to room temperature before weighing again.
2.7.5. Cap tube and save residual dried material for subsequent combustion analysis for protein (see below).
2.7.6. Store in a desiccator for further analysis.

2.8. Calculation of Moisture Content.

$$Moisture = \frac{\begin{matrix}( wt . tube + tissue as is - wt . tube ) - \\ ( wt . tube + tissue dry - wt . tube )\end{matrix}}{( wt . tube + tissue as is - wt . tube )} \times 100$$

2.9. Whole Seed Moisture Calibrations.

Whole seed moisture calibrations for the SS-NIR were developed according to the methods described below. Pods were harvested from Jack, 93686 and 93Y21 soybeans between the R7 and R8 stage of development (i.e., yellow to brown pod stage) when the soybeans had moisture contents of below 20%. Beans were removed from the pods and their weight was measured and recorded to 0.0001 g accuracy prior to spectral capture using the SS-NIR instrument. The beans were then subjected to controlled drying (@105° C. in a forced draft oven for short periods of time) to attain a broad range of moisture contents before repeat weighing and spectral capture. A final dry weight for each bean was obtained after drying in a forced draft oven at 105° C. for 18 h. Moisture content was calculated as follows:

$$moisture content = \frac{\begin{matrix}( ( wt . tube + bean as {is}^{*} - wt . tube ) - \\ ( wt . tube + oven dried bean - wt . tube ) )\end{matrix}}{{( wt . of bean as is - wt . tube )}^{*}} \times 100$$

$${*}^{} at time of spectral capture .$$

Alternatively, mature soybeans were placed in 5×6″ aluminum foil trays in 1 gallon ZipLock® plastic bags. The relative humidity of the atmosphere within the bags was controlled by adding a second foil pan in which either a layer of self-indicating DrieRite desiccant (W. A. Hammond Inc; Xenia OH) or a saturated aqueous solution of sodium chloride (200 g NaCl in ⅓rd pan depth of water). A third bag containing seed but without any atmospheric moisture control was also set up. The beans were exposed to the controlled moisture atmospheres for one month prior to weighing followed by immediate spectral capture on the SS-NIR. In order to maintain the individual identity of each bean after scanning they were placed into 16×125 mm Pyrex® glass tubes. The beans were then dried according to AOCS Official Method Ac 2-41 (modified for small samples) as follows:
2.9.1. Place bean into a pre-weighed (and recorded) 16×125 mm glass tube and weigh again; record weights to an accuracy of 0.1 mg.
2.9.2. Place samples into a forced air oven preheated to 130° C.
2.9.3. Allow material to dry for 3 h.
2.9.4. Remove tubes into a desiccator cabinet and allow to come to room temperature before weighing again.

$$moisture content = \frac{\begin{matrix}( ( wt . tube + bean as {is}^{*} - wt . tube ) - \\ ( wt . tube + oven dried bean - wt . tube ) )\end{matrix}}{( wt . tube bean as {is}^{*} - wt . tube )} \times 100$$

$${*}^{} at time of spectral capture .$$

Predictive spectral models for seed moisture content were developed by combining the spectral information with the measured moisture contents for each bean.

2.10. Protein Analysis.

Protein contents were estimated by combustion analysis of the oven dried powders described above. Analysis was performed on a Flash™ 1112 EA combustion analyzer (commercially available from Thermo Scientific) running in the Nitrogen, Carbon, Sulfur (NCS) mode. Samples of oven dried (according to AOCS Official Method Ba 2a-38 as described above) powdered samples, 4-8 mg (NCS Mode), weighed to an accuracy of 0.001 mg on a Mettler-Toledo MX5 microbalance were used for analysis. Protein contents were calculated by multiplying % N, determined by the analyzer, by 6.25. All samples were run in duplicate. If the difference between the protein contents of the replicate samples was >5% of the mean value, additional replicates were analyzed. Final protein contents were measured on a dry weight basis and adjusted to the desired moisture content.

Alternatively, the Thermo Scientific™ Flash™ 1112 EA combustion analyzer was run in N-protein mode, according to the Manufacturer's instructions, using aspartic acid as the standard. Samples of oven dried (according to AOCS Official Method Ba 2a-38 described above). The powdered samples, 30-40 mg, weighed to an accuracy of 0.001 mg on a Mettler-Toledo MX5 microbalance were used for analysis. Protein contents were calculated by multiplying % N, determined by the analyzer, by 6.25. All samples were run in duplicate. If the difference between the protein contents of the replicate samples was >5% of the mean value, additional replicates (if material was available) were analyzed. Final protein contents were measured on a dry weight basis and adjusted to the desired moisture content.

2.11. NMR based Analysis of Seed Oil Content.

Whole seed and powdered sample oil contents were determined using a Maran Ultra NMR analyzer (Resonance Instruments Ltd, Whitney, Oxfordshire, UK). Samples (either individual intact soy seed or batches ˜200 mg of ground soy powder) were placed into pre-weighed 2 ml polypropylene tubes (Corning Inc, Corning NY, USA; part #430917) previously labeled with unique bar code identifiers. Samples were placed into 96 place carriers and processed through the following series of steps by an Adept Cobra 600 SCARA robotic system.
2.11.1. Pick up tube using robotic arm fitted with a vacuum pickup device.
2.11.2. Read bar code.
2.11.3. Expose tube to antistatic device to ensure powdered samples do not adhere to the tube walls.
2.11.4. Weigh sample, to 0.1 mg accuracy.
2.11.5. NMR reading; measured as the intensity of the proton spin echo 1 msec after a 22.9 MHz signal was applied to the sample. Data was collected for 32 NMR scans per sample.
2.11.6. Return tube to rack.
2.11.7. Repeat process with next tube.

Bar codes, seed weights and NMR readings were recorded by a computer connected to the system.

Seed oil content was calculated as follows:

$$\% oil ( \% wt . basis ) = \frac{[ ( NMR signal / seed wt . ( g ) ) - 70.58 ]}{351.45}$$

Calibration parameters were determined by precisely weighing samples of soy oil (ranging from 0.0050 to 0.0700 g at approximately 0.0050 g intervals; weighed to an accuracy of 0.0001 g) into the polypropylene tubes (see above) and subjecting them to NMR analysis. A calibration curve of oil content (% seed wt. basis; assuming a standard seed weight of 0.1500 g) to NMR value was established.

Analytical Methods for bulk samples.

2.12. Moisture Determinations and Creation of Bulk Bean Moisture Calibrations.

Field or greenhouse-grown soybeans varieties 93686 (U.S. Pat. No. 6,610,910) and 93Y21 (commercially available from Pioneer Hybrid International) were harvested and the moisture calibrations for the FT-NIR were developed according to the methods described below. Pods were harvested from soybean plants between the R7 and R8 stage of development (i.e., yellow to brown pod stage) when the soybeans had moisture contents below 50 wt. %. Beans were removed from the pods and were separated into groups of approximately 25 g based on their state of maturity. The weight of the bean sample was measured and recorded to 0.0001 g accuracy, prior to FT-NIR spectral capture in a 54 mm spinning cup. The beans were then placed into 5″×6″ foil trays and positioned in a laminar flow hood to dry at room temperature for varying times. After the seed had undergone measurable drying, the beans were weighed again and rescanned. This process was repeated until no further weight loss was observed. The samples were then taken to complete dryness using AOCS Official Method Ac 2-41, and were allowed to come to room temperature in a desiccator prior to weighing and rescanning on the FT-NIR. Moisture content was calculated as follows:

$$Moisture Content = \frac{( wt . of beans as {is}^{*} - wt . of oven dried beans )}{wt . of beans as {is}^{*}} \times 100$$

$${*}^{} at time of spectral capture .$$

Alternatively, mature soybeans were placed in 5″×6″ aluminum foil trays in 1 gallon ZipLock® brand plastic sealable bags. The relative humidity of the atmosphere within the bags was controlled by adding a second foil pan which contained either a layer of self-indicating DrieRite desiccant (W. A. Hammond Inc; Xenia OH), or a saturated aqueous solution of sodium chloride. A third bag containing the seed tray but without any atmospheric moisture control was also set up. The beans were exposed to the controlled moisture atmospheres for one month prior to weighing followed by immediate spectral capture on the FT-NIR. The beans were then dried according to AOCS Official Method Ac 2-41, as described above, and scanned again. Predictive spectral models for seed moisture content were developed by combining the spectral information with the measured moisture contents for each bean sample.

2.13. Sample Grinding and Preparation for Bulk Reference Chemistry.

Seventy-five gram batches of beans were ground to a powder in a Foss Knifetec 1095 grinder (commercially available from FOSS North America, Eden Prairie, MN). The grinding chamber was cooled prior to and during the process by a circulating chiller set to 14° C. Samples were ground for 2×10 second bursts using a standard rotor blade. The ground sample was transferred to a 6″ diameter stainless steel sieve (1 mm mesh) and sifted (resulting in less than 2% loss of material) before being placed into an airtight sample cup. The sample chamber and blade were cleaned thoroughly with a soft brush and pneumatic air prior to introduction of the next sample. Sample cups were stored at room temperature in the dark prior to further analysis.

In later experiments the grinding method was modified to remove the need for sifting. Under these conditions the beans were ground for 6×10 second bursts under the conditions described above. The chamber was opened between each burst and material adhering to the chamber wall was scraped off with a plastic spatula and returned to the center of the chamber. This grinding protocol was found to create a powdered sample that would pass through a US No 20 mesh sieve with no loss and be more suitable for crude fat extraction.

2.14. Crude Protein Analysis.

Crude Protein contents were measured by combustion analysis of the oven dried powders described above in accordance to AOCS Official Method Ba 4e-93. Analyses were either performed by a contract research organization according to Industry Standard methods for soybean or otherwise as described herein. The protocols are essentially the same as those used for single seed (Example 2.10) but have been modified to accommodate a larger sample size. Analysis was performed on a Thermo Scientific™ Flash™ 1112 EA combustion analyzer running in the N-Protein mode, following the manufacturer's recommendations. Samples of the dried powders, 30-40 mg, weighed to an accuracy of 0.001 mg on a Mettler-Toledo MX5 microbalance, were used for analysis. Protein contents were calculated by multiplying % N, determined by the analyzer, by 6.25. Final protein contents were expressed on a 13% moisture corrected basis. All samples were run in duplicate and further replication was performed if the difference between the replicate samples was >5% of the mean value.

2.15. Crude Fat/Oil Analysis

Crude Fat/Oil determinations were performed according to AOCS Official Method Ba 3-38. Analyses were either performed a commercial service laboratory (Eurofins Scientific Inc., Des Moines, IA 50321) or done using a Foss SoxTec 8000 Extraction Unit (commercially available from Foss Analytical AB Höganäs, Sweden), according to the manufacturers recommendations (Application Note_AN 3487), with slight modification. Powder samples taken at the time of analysis were subjected to moisture determinations using AOCS Official Method Ba 2a-38, as described above. Final crude oil contents were expressed on a 13% moisture corrected basis. All samples were run in duplicate.

### Example 3

Single Seed Spectral Analysis of Soybeans and Development of Accurate Measurements

Seed with a high degree of compositional diversity selected from the materials described in Example 1 were analyzed on a proprietary Single Seed Near Infrared (SS-NIR) spectrometer (U.S. Pat. No. 7,274,456 B2, issued Sep. 25, 2007; U.S. Pat. No. 7,508,517B2, issued Mar. 24, 2009). Briefly, in the SS-NIR system an individual bean was introduced into the analytical cell where it was illuminated from all points in three dimensions. The seed was tumbled with an air stream, within an approximated integrating sphere constructed from a 16-mm-diameter quartz cup coated with 6080 white reflectance coating (Labsphere, North Hutton, NH). Illumination was provided through 12 optical fibers, connected to four 20 Watt 8211-002 light bulbs (Welch Allyn, Skaneateles Falls, NY), the ends of which were incorporated into the cell cover. The reflected spectral region from 904 to 1686 nm was collected through the apex of the cover of the sampling cell by an NIR512 spectrometer (Control Development, South Bend, IN). Each seed was scanned for 6 seconds to collect spectra that were optimized for maximal signal to noise ratio. Spectral quality was monitored during each sample scan by regularly checking the Root Mean Square (RMS) noise of the 100% lines. The 100% lines were computed by the ratio between every two spectra of the triplicate measurement for each sample. Under ideal, noise-free conditions, the 100% lines would be straight horizontal lines at zero absorbance units (AU) since all replicate spectra come from the same sample providing the same spectral features. To minimize instrumental drift, system noise, seed condition and other environmental changes, noise and off-sets were observed in the actual 100% lines. After scanning, the seed was ejected from the sample cup and transferred to an indexed sample tray. The individual identity of each seed was therefore preserved, facilitating instrument calibration.

Separate calibration models were generated for every constituent of interest using Partial Least Square (PLS) analysis coupled with an optimized number of latent variables, spectral range and spectral preprocessing, before being applied to online/offline compositional analysis of the individual seed components, such as the sucrosyl-oligosaccharides. The optimized number of latent variables, spectral range and spectral preprocessing were determined by analyzing the training and monitoring subset from the calibration data where the calibration performance reached an optimum level, in terms of Root Mean Square Error of Calibration (RMSEC) and Root Mean Square Error of Cross Validation (RMSECV). Taking stachyose as an example, the optimized number of latent variables was determined by the co-constituents with the least distinct spectral features. The calibration model used two components: the fewest latent variables and the most stachyose-related information. The balance of compromising these two components is dependent on the distinctness of the pure component spectrum for stachyose within the spectral matrix. For those co-constituents with distinct spectra, such as oil, a few PLS latent variables were used to capture enough information. More PLS latent variables were needed to separate stachyose from the co-constituents such as sucrose and raffinose which are chemically related and therefore give a high degree of spectral overlap. The optimized spectral range for stachyose was in the vicinity of 1000, 1200, 1380 and 1460 nm. These wavelengths enabled stachyose to be measured distinctly from the other constituents of the soybean seed. After the spectra were preprocessed for multiplicative scatter corrections, Savitsky-Golay derivatives and polynomial smoothing were applied in the spectral region between 904-1540 nm. The number of latent variables was determined as the fewest number of latent variables that resulted in an optimal calibration/cross validation accuracy as determined by the RMSEC (Root Mean Square Error of Calibration) and RMSECV (Root Mean Square Error of Cross Validation), respectively. The optimum calibration model was selected based on the R2 (statistical measure of how close the predicted and reference chemistry data are fitted by the regression line), RMSEC (Root Mean Square Error of Calibration) and RMSECV (Root Mean Square Error of Cross Validation) statistics.

TABLE 3
Statistics for various seed component SS-NIR calibrations.
The number of reference chemistry measurements used to develop
the calibrations for each constituent are shown in column n.
The dynamic range in composition underpinning each constituent
calibration is shown in the range column. Under these conditions,
stachyose could be detected as low as 0.05 wt. %.

| | | Range | | | |
|---|---|---|---|---|---|
| Constituent | n | (wt. %-wt. %) | R² | RMSEC | RMSECV |
| weight | 3096 | 0.09-0.30 | 0.99 | 4.2 mg | 4.4 mg |
| moisture | 618 | 5.4-15.7 | 0.94 | 0.51% | 0.54% |
| protein | 563 | 32.9-49.6 | 0.92 | 0.89% | 1.04% |
| oil | 1608 | 15.2-26.4 | 0.98 | 0.32% | 0.33% |
| oleic | 2725 | 12.8-90.1 | 0.99 | 2.80% | 2.83% |
| linoleic | 2725 | 1.1-61.8 | 0.98 | 2.67% | 2.72% |
| linolenic | 2725 | 1.1-12.7 | 0.92 | 0.81% | 0.86% |
| stearic | 2725 | 2.1-7.3 | 0.78 | 0.45% | 0.47% |
| palmitic | 2725 | 2.2-13.4 | 0.91 | 0.60% | 0.65% |
| stachyose | 730 | 0.05-5.5 | 0.91 | 0.52% | 0.56% |
| sucrose | 952 | 2.51-9.88 | 0.81 | 0.58% | 0.67% |
| total | 670 | 5.6-14.1 | 0.87 | 0.55% | 0.65% |
| carbohydrate | | | | | |

Stachyose measurements by SS-NIR and reference chemistry methods are shown in Table 4

TABLE 4
Stachyose contents of 20 individual T1 seed from Soil
2-7879-1-2-1 event. Each seed was analyzed by SS-NIR and
was then ground and the stachyose content was measured
using reference chemistry. Null segregants were identified
(bold values underlined) based on the reference chemistry
stachyose values. The mean and standard deviation (SD) values
for the null and transgenic positive seed are also presented.

| | | Stachyose | Single |
|---|---|---|---|
| | | by | Seed |
| | | Reference | Stachyose |
| Seed ID | Event ID | Chemistry | by NIR |
| 1 s | Soil 2 AFS 7879-1-2-1 T1 seed | 4.09 | 4.53 |
| 2 s | Soil 2 AFS 7879-1-2-1 T1 seed | 0.22 | 0.83 |
| 3 s | Soil 2 AFS 7879-1-2-1 T1 seed | 0.31 | 0.44 |
| 4 s | Soil 2 AFS 7879-1-2-1 T1 seed | 0.22 | 0.56 |
| 5 s | Soil 2 AFS 7879-1-2-1 T1 seed | 0.19 | 0.24 |
| 6 s | Soil 2 AFS 7879-1-2-1 T1 seed | 0.29 | 0.82 |
| 7 s | Soil 2 AFS 7879-1-2-1 T1 seed | 0.18 | 0.46 |
| 8 s | Soil 2 AFS 7879-1-2-1 T1 seed | 0.42 | 0.33 |
| 9 s | Soil 2 AFS 7879-1-2-1 T1 seed | 3.91 | 4.55 |
| 10 s | Soil 2 AFS 7879-1-2-1 T1 seed | 0.09 | 0.37 |
| 11 s | Soil 2 AFS 7879-1-2-1 T1 seed | 3.61 | 3.83 |
| 12 s | Soil 2 AFS 7879-1-2-1 T1 seed | 0.08 | −0.01 |
| 13 s | Soil 2 AFS 7879-1-2-1 T1 seed | 0.24 | 0.49 |
| 14 s | Soil 2 AFS 7879-1-2-1 T1 seed | 0.14 | 0.31 |
| 15 s | Soil 2 AFS 7879-1-2-1 T1 seed | 0.18 | 0.20 |
| 16 s | Soil 2 AFS 7879-1-2-1 T1 seed | 0.22 | 0.97 |
| 17 s | Soil 2 AFS 7879-1-2-1 T1 seed | 3.82 | 3.80 |
| 18 s | Soil 2 AFS 7879-1-2-1 T1 seed | 0.28 | 0.65 |
| 19 s | Soil 2 AFS 7879-1-2-1 T1 seed | 3.67 | 4.09 |
| 20 s | Soil 2 AFS 7879-1-2-1 T1 seed | 0.41 | 0.70 |
| | Mean Soil 2 AFS 7879-1-2-1 | | |
| | Null | 3.82 | 4.16 |
| | SD | 0.19 | 0.37 |
| | Mean Soil 2 AFS 7879-1-2-1 | | |
| | Pos | 0.23 | 0.49 |
| | SD | 0.10 | 0.27 |

The construct used to create the Soil 92-2499.1.1.1 event contained transgenic components a Yarrowia lipolytica, Diacyl glycerol transferase-2 (DGAT-2) under the control of the strong seed specific β-Conglycinin promoter, the soy transcription factor ODP1, a sucrose transporter SUT4 and a GAS suppression component under the seed-specific kTi promoter. Seed from Soil 92 events have elevated oil, protein, oleic and stearic acids in conjunction with decreased linoleic and linolenic acids, sucrose and total soluble sugars. Lower stachyose contents would be expected and were measured using SS-NIR. The compositions of 36 T1 seed from the Soil 92-2499.1.1.1 event measured by NIR are given in Table 5A and 5B.

TABLE 5A
Single seed compositions for 36 T1 seed from Soil 92-2499.1.1.1
event. The composition of the seeds provided a distinct finger
print that was used to discriminate between transgenic positive
and null-segregant seed. Seed identified as null segregants
are indicated in bold type and underlined.

| | | | | | | Total |
|---|---|---|---|---|---|---|
| | | | | | | sol- |
| | | Stach- | | Pro- | Su- | uble |
| Seed ID | Event ID | yose | Oil | tein | crose | CBH |
| 14SN30- | Soil 92 SOY | 3.2 | 28.1 | 36.2 | 4.4 | 8.5 |
| 829 | 2499.1.1.1 | | | | | |
| 14SN30- | Soil 92 SOY | 3 | 25 | 43.6 | 4.2 | 7.8 |
| 830 | 2499.1.1.1 | | | | | |
| 14SN30- | Soil 92 SOY | 3.1 | 25.2 | 42.7 | 3.3 | 7.4 |
| 831 | 2499.1.1.1 | | | | | |
| 14SN30- | Soil 92 SOY | 4.4 | 19 | 40.1 | 6.1 | 11.5 |
| 832 | 2499.1.1.1 | | | | | |
| 14SN30- | Soil 92 SOY | 4.2 | 15.6 | 42.8 | 6.8 | 12.1 |
| 833 | 2499.1.1.1 | | | | | |
| 14SN30- | Soil 92 SOY | 3.3 | 28.2 | 46 | 3 | 7.4 |
| 834 | 2499.1.1.1 | | | | | |
| 14SN30- | Soil 92 SOY | 4.3 | 18.9 | 40.1 | 6.2 | 11.9 |
| 835 | 2499.1.1.1 | | | | | |
| 14SN30- | Soil 92 SOY | 3.1 | 27.1 | 42.3 | 2.9 | 6.6 |
| 836 | 2499.1.1.1 | | | | | |
| 14SN30- | Soil 92 SOY | 3.9 | 19.4 | 37.1 | 6.8 | 12.3 |
| 837 | 2499.1.1.1 | | | | | |
| 14SN30- | Soil 92 SOY | 2.4 | 25.5 | 43.4 | 4.3 | 7.3 |
| 838 | 2499.1.1.1 | | | | | |
| 14SN30- | Soil 92 SOY | 4.1 | 18 | 41.7 | 7 | 11.9 |
| 839 | 2499.1.1.1 | | | | | |
| 14SN30- | Soil 92 SOY | 3.2 | 27.1 | 40.9 | 3.4 | 7.2 |
| 840 | 2499.1.1.1 | | | | | |
| 14SN30- | Soil 92 SOY | 3.2 | 26.2 | 42.8 | 3.4 | 7.2 |
| 841 | 2499.1.1.1 | | | | | |
| 14SN30- | Soil 92 SOY | 4.2 | 19.9 | 38.9 | 6.4 | 12 |
| 842 | 2499.1.1.1 | | | | | |
| 14SN30- | Soil 92 SOY | 2.7 | 25.7 | 45.5 | 2.9 | 6.1 |
| 843 | 2499.1.1.1 | | | | | |
| 14SN30- | Soil 92 SOY | 3.4 | 26.6 | 40.7 | 2.7 | 6.6 |
| 844 | 2499.1.1.1 | | | | | |
| 14SN30- | Soil 92 SOY | 3.4 | 25 | 40.3 | 3 | 7.1 |
| 845 | 2499.1.1.1 | | | | | |
| 14SN30- | Soil 92 SOY | 3.8 | 27.5 | 41.8 | 3.5 | 8.1 |
| 846 | 2499.1.1.1 | | | | | |
| 14SN30- | Soil 92 SOY | 4 | 17.3 | 41.9 | 6.4 | 11.7 |
| 847 | 2499.1.1.1 | | | | | |
| 14SN30- | Soil 92 SOY | 4.1 | 20.2 | 37.8 | 6.7 | 12.1 |
| 848 | 2499.1.1.1 | | | | | |
| 14SN30- | Soil 92 SOY | 2.7 | 25.6 | 43.9 | 4.2 | 7 |
| 849 | 2499.1.1.1 | | | | | |
| 14SN30- | Soil 92 SOY | 2.5 | 28.8 | 41.4 | 3.6 | 6.6 |
| 850 | 2499.1.1.1 | | | | | |
| 14SN30- | Soil 92 SOY | 2.9 | 27.7 | 42.6 | 3.8 | 7.2 |
| 851 | 2499.1.1.1 | | | | | |
| 14SN30- | Soil 92 SOY | 2.7 | 25 | 44.3 | 4 | 7.2 |
| 852 | 2499.1.1.1 | | | | | |
| 14SN30- | Soil 92 SOY | 3.3 | 25.2 | 44.7 | 3.3 | 6.7 |
| 853 | 2499.1.1.1 | | | | | |
| 14SN30- | Soil 92 SOY | 4.3 | 19.6 | 37.4 | 7.1 | 13 |
| 854 | 2499.1.1.1 | | | | | |
| 14SN30- | Soil 92 SOY | 3.5 | 23.4 | 43 | 4.4 | 8.1 |
| 855 | 2499.1.1.1 | | | | | |
| 14SN30- | Soil 92 SOY | 4.7 | 19.8 | 38.5 | 7.3 | 12.7 |
| 856 | 2499.1.1.1 | | | | | |
| 14SN30- | Soil 92 SOY | 3 | 25.3 | 43.4 | 3.7 | 7.2 |
| 857 | 2499.1.1.1 | | | | | |
| 14SN30- | Soil 92 SOY | 3.4 | 25.8 | 40.6 | 4 | 7.6 |
| 858 | 2499.1.1.1 | | | | | |
| 14SN30- | Soil 92 SOY | 2.9 | 28.6 | 40.8 | 3.9 | 7.2 |
| 859 | 2499.1.1.1 | | | | | |
| 14SN30- | Soil 92 SOY | 3.3 | 26.6 | 39.5 | 4.1 | 8.1 |
| 860 | 2499.1.1.1 | | | | | |
| 14SN30- | Soil 92 SOY | 2.7 | 28.4 | 40 | 4.1 | 7.3 |
| 861 | 2499.1.1.1 | | | | | |
| 14SN30- | Soil 92 SOY | 2.9 | 25.1 | 43.7 | 3.9 | 7.4 |
| 862 | 2499.1.1.1 | | | | | |
| 14SN30- | Soil 92 SOY | 3.5 | 24 | 42 | 4 | 7.8 |
| 863 | 2499.1.1.1 | | | | | |
| 14SN30- | Soil 92 SOY | 3.8 | 19.9 | 41.8 | 4.5 | 8.9 |
| 864 | 2499.1.1.1 | | | | | |
| | Mean Soil 92 | 4.18 | 18.87 | 39.83 | 6.48 | 11.83 |
| | 2499.1.1.1 | | | | | |
| | T1 Null | | | | | |
| | SD | 0.25 | 1.40 | 2.02 | 0.76 | 1.06 |
| | Mean 92 | 3.08 | 26.27 | 42.24 | 3.68 | 7.31 |
| | 2499.1.1.1 | | | | | |
| | T1 Pos | | | | | |
| | SD | 0.34 | 1.49 | 2.13 | 0.51 | 0.56 |
| | Delta | −1.10 | 7.40 | 2.42 | −2.80 | −4.52 |

TABLE 5B
Single seed compositions (fatty acid methyl esters as a percent of the sum of all
of the fatty acid methyl esters) for 36 T1 seed from Soil 92-2499.1.1.1 events
(as in Table 5A). The composition of the seeds provided a distinct finger print
that was used to discriminate between transgenic positive and null-segregant seed.
Seed identified as null segregants are indicated in bold type and underlined.

| Seed ID | Event ID | Oleic | Linolenic | Palmitic | Stearic | Linoleic | Weight | Moisture |
|---|---|---|---|---|---|---|---|---|
| 14SN30- | Soil 92 SOY | 29.1 | 5.5 | 12.7 | 6.3 | 46.3 | 0.2 | 8.3 |
| 829 | 2499.1.1.1 | | | | | | | |
| 14SN30- | Soil 92 SOY | 28.6 | 5.3 | 12.8 | 6.1 | 48.2 | 0.3 | 8.1 |
| 830 | 2499.1.1.1 | | | | | | | |
| 14SN30- | Soil 92 SOY | 30.2 | 5.2 | 12.2 | 6.2 | 45.9 | 0.2 | 8.1 |
| 831 | 2499.1.1.1 | | | | | | | |
| 14SN30- | Soil 92 SOY | 20.4 | 9.9 | 11.3 | 4 | 54.9 | 0.3 | 8.9 |
| 832 | 2499.1.1.1 | | | | | | | |
| 14SN30- | Soil 92 SOY | 17.4 | 12 | 12 | 4.4 | 54.4 | 0.2 | 8.9 |
| 833 | 2499.1.1.1 | | | | | | | |
| 14SN30- | Soil 92 SOY | 38.2 | 7.4 | 12.9 | 9.3 | 34.7 | 0.2 | 7.2 |
| 834 | 2499.1.1.1 | | | | | | | |
| 14SN30- | Soil 92 SOY | 17.4 | 10.9 | 11.7 | 4.2 | 57.2 | 0.2 | 8.8 |
| 835 | 2499.1.1.1 | | | | | | | |
| 14SN30- | Soil 92 SOY | 35.1 | 4.8 | 12.2 | 7.4 | 40.8 | 0.2 | 8.1 |
| 836 | 2499.1.1.1 | | | | | | | |
| 14SN30- | Soil 92 SOY | 16.8 | 11 | 10.9 | 4.2 | 58.6 | 0.2 | 8.7 |
| 837 | 2499.1.1.1 | | | | | | | |
| 14SN30- | Soil 92 SOY | 29.6 | 4.2 | 12.2 | 6 | 48.6 | 0.3 | 8.1 |
| 838 | 2499.1.1.1 | | | | | | | |
| 14SN30- | Soil 92 SOY | 15.7 | 11 | 12 | 4.2 | 57.4 | 0.2 | 9.4 |
| 839 | 2499.1.1.1 | | | | | | | |
| 14SN30- | Soil 92 SOY | 32.9 | 4.4 | 12.4 | 6.8 | 42.7 | 0.2 | 7.9 |
| 840 | 2499.1.1.1 | | | | | | | |
| 14SN30- | Soil 92 SOY | 34 | 3.9 | 12.1 | 7 | 43.6 | 0.2 | 8.2 |
| 841 | 2499.1.1.1 | | | | | | | |
| 14SN30- | Soil 92 SOY | 18.2 | 9.8 | 11.6 | 4.3 | 56.8 | 0.2 | 9 |
| 842 | 2499.1.1.1 | | | | | | | |
| 14SN30- | Soil 92 SOY | 34 | 3.7 | 11.7 | 6.7 | 44.3 | 0.3 | 7.7 |
| 843 | 2499.1.1.1 | | | | | | | |
| 14SN30- | Soil 92 SOY | 33.3 | 5 | 13 | 7.5 | 40.8 | 0.2 | 8.2 |
| 844 | 2499.1.1.1 | | | | | | | |
| 14SN30- | Soil 92 SOY | 29.8 | 7.2 | 11.6 | 6.2 | 45.4 | 0.2 | 8.8 |
| 845 | 2499.1.1.1 | | | | | | | |
| 14SN30- | Soil 92 SOY | 32.1 | 7.6 | 12.1 | 7.5 | 42.3 | 0.2 | 7.5 |
| 846 | 2499.1.1.1 | | | | | | | |
| 14SN30- | Soil 92 SOY | 17.9 | 11.5 | 11 | 4.1 | 56.1 | 0.3 | 8.7 |
| 847 | 2499.1.1.1 | | | | | | | |
| 14SN30- | Soil 92 SOY | 16.7 | 10.4 | 11.8 | 4.3 | 57.8 | 0.2 | 8.9 |
| 848 | 2499.1.1.1 | | | | | | | |
| 14SN30- | Soil 92 SOY | 30.5 | 4.4 | 11.8 | 5.5 | 46.6 | 0.3 | 8.4 |
| 849 | 2499.1.1.1 | | | | | | | |
| 14SN30- | Soil 92 SOY | 36 | 3 | 11.4 | 6.3 | 40.9 | 0.2 | 7.6 |
| 850 | 2499.1.1.1 | | | | | | | |
| 14SN30- | Soil 92 SOY | 32.3 | 5.9 | 11.1 | 6.5 | 43.5 | 0.2 | 7.2 |
| 851 | 2499.1.1.1 | | | | | | | |
| 14SN30- | Soil 92 SOY | 32.1 | 4.6 | 11.2 | 6 | 44.6 | 0.2 | 8.3 |
| 852 | 2499.1.1.1 | | | | | | | |
| 14SN30- | Soil 92 SOY | 37.7 | 5.3 | 11.7 | 6.3 | 37.6 | 0.2 | 8.1 |
| 853 | 2499.1.1.1 | | | | | | | |
| 14SN30- | Soil 92 SOY | 18.5 | 10.4 | 10.4 | 4 | 56 | 0.2 | 8.9 |
| 854 | 2499.1.1.1 | | | | | | | |
| 14SN30- | Soil 92 SOY | 31.7 | 5 | 11.2 | 5.9 | 43.6 | 0.2 | 8.8 |
| 855 | 2499.1.1.1 | | | | | | | |
| 14SN30- | Soil 92 SOY | 20.2 | 9.2 | 11 | 4.2 | 53.6 | 0.2 | 9.2 |
| 856 | 2499.1.1.1 | | | | | | | |
| 14SN30- | Soil 92 SOY | 32.1 | 4.7 | 11.7 | 5.9 | 44 | 0.2 | 8.5 |
| 857 | 2499.1.1.1 | | | | | | | |
| 14SN30- | Soil 92 SOY | 32.2 | 6.1 | 11.3 | 5.6 | 42.7 | 0.2 | 8.1 |
| 858 | 2499.1.1.1 | | | | | | | |
| 14SN30- | Soil 92 SOY | 35.5 | 3.9 | 11.8 | 6.9 | 40.5 | 0.2 | 8 |
| 859 | 2499.1.1.1 | | | | | | | |
| 14SN30- | Soil 92 SOY | 31.7 | 5.2 | 11.5 | 5.7 | 44 | 0.2 | 8.6 |
| 860 | 2499.1.1.1 | | | | | | | |
| 14SN30- | Soil 92 SOY | 34.8 | 3.7 | 12.2 | 6.7 | 39.8 | 0.2 | 8.1 |
| 861 | 2499.1.1.1 | | | | | | | |
| 14SN30- | Soil 92 SOY | 32 | 4.9 | 11.6 | 6.1 | 44.2 | 0.2 | 8.5 |
| 862 | 2499.1.1.1 | | | | | | | |
| 14SN30- | Soil 92 SOY | 32.6 | 5.4 | 11.8 | 6 | 42.6 | 0.2 | 9.1 |
| 863 | 2499.1.1.1 | | | | | | | |
| 14SN30- | Soil 92 SOY | 20.9 | 10.1 | 9.9 | 3.5 | 54.5 | 0.2 | 9 |
| 864 | 2499.1.1.1 | | | | | | | |
| | Mean | 18.19 | 10.56 | 11.24 | 4.13 | 56.12 | 0.22 | 8.95 |
| | Null | | | | | | | |
| | SD | 1.67 | 0.81 | 0.67 | 0.24 | 1.60 | 0.04 | 0.21 |
| | Mean | 32.72 | 5.05 | 11.93 | 6.50 | 43.13 | 0.22 | 8.14 |
| | Pos | | | | | | | |
| | SD | 2.51 | 1.14 | 0.54 | 0.81 | 3.12 | 0.04 | 0.46 |
| | Delta | 14.53 | −5.51 | 0.69 | 2.37 | −12.99 | 0.00 | −0.81 |

From the results presented in Tables 5A and 5B, all components except for stachyose were measured at the expected values. Stachyose contents were lowered by an average of 1.1% (percentage points), instead of the expected at least 3% (percentage points) indicating that the construct did not produce the expected composition. Additional 5 components were used to assist in distinguishing transgenic positive from null-segregant seeds where transgenic changes are subtle. For example, seeds 14SN30-846 and 14SN30-864 each had a stachyose content of 3.8%, which was the threshold used for discriminating between transgenic positive and null-segregant seed. By inspecting the other components (27.5% oil, 41.8% protein, 32.1% oleic, 3.5% sucrose, 8.1% soluble carbohydrates, 7.5% linolenic acid) it was apparent that 14SN30-846 was a transgenic positive seed and that 14SN30-864 (19.9% oil, 41.8% protein, 20.9% oleic, 4.5% sucrose, 8.9% soluble carbohydrates, 10.1% linolenic acid) was a null segregant.

The methods are also suitable for screening material generated in crossing experiments designed to introgress the low sucrosyl-oligosaccharide/high oil transgenes into elite soybean backgrounds. In this example pollen from heterozygous BC1F1 plants from the Oil 119 event (segregating for the transgenes for the low sucrosyl-oligosaccharide/high oil traits) was used to fertilize the emasculated receptive flowers of three elite soybean varieties. The cross fertilized plants were grown to maturity and the resulting BC2F1 seed harvested from the cross-pollinated flowers were analyzed by SS-NIR. This SS-NIR analysis allowed the non-destructive identification of seed carrying the desired transgenic phenotype (i.e., those displaying a low stachyose and high oil phenotype). These positively identified seed were grown and pollen from these plants was again used to pollinate the emasculated receptive flowers of the same three elite soybean varieties. The results in Table 6 show the composition of mature seed harvested after three rounds of backcrossing onto the recurrent female elite parent. In most cases a threshold value for stachyose content of 2.0% was used to differentiate between the wild type seeds (>2.0% stachyose; indicated with bold type and underlined) and those (<<2.0%) that resulted from successful transgene introgressions (Transgenic pos). Further confirmation of successful transgene introgressions was provided by the other constituents influenced by the transgenic traits i.e., elevated oil, protein and oleic acid, reduced levels of sucrose, total soluble carbohydrates and linolenic acid. Transgenic hybrid seed could be identified using SS-NIR by combining the low stachyose phenotype (of ≤0.32%) with a combination of high oil, high protein, high oleic acid, low sucrose, low total soluble carbohydrates and low linolenic acid phenotypes (dependent on the background) that result from the expression of the high oil components of the transgenic cassette. Soybeans seeds of varied genetic backgrounds outside of those used to generate the calibration curves could be successfully identified as containing introgressed transgenes.

TABLE 6A
SS-NIR compositions (oil/protein/carbohydrate (CBH)) for segregating
seed resulting from backcrosses of an Oil 119 event. The construct
used to create the Oil119 event contained the following transgenic
components, a modified Glycine max diacyl glycerol transferase-1
(DGAT-1) under the control of the seed specific S-albumen promoter,
a sucrose transporter SUT4 and a GAS 1, 2, 3 suppression component
under the control of the strong seed specific β-conglycinin
promoter. This construct conferred a low stachyose/high oil phenotype
in three elite female soybean lines.

| | | | | | | | Total |
|---|---|---|---|---|---|---|---|
| | | | | | | | sol- |
| | Plant | SSNIR | Stach- | | Pro- | Su- | uble |
| Female/Male | ID | zyg call | yose | Oil | tein | crose | CBH |
| 92Y51/ | 4 | Wild | 2.50 | 19.32 | 45.47 | 7.42 | 11.55 |
| BC75638705 | | type | | | | | |
| 92Y51/ | 3 | Wild | 2.61 | 19.08 | 45.93 | 7.32 | 11.65 |
| BC75638705 | | type | | | | | |
| 92Y51/ | 2 | TG | 0.17 | 24.32 | 46.98 | 6.31 | 8.33 |
| BC75638705 | | POS | | | | | |
| 92Y51/ | 5 | TG | 0.25 | 24.61 | 46.60 | 7.54 | 7.97 |
| BC75638705 | | POS | | | | | |
| 92Y51/ | 1 | TG | 0.28 | 24.01 | 46.63 | 7.40 | 8.46 |
| BC75638705 | | POS | | | | | |
| | Wild | Mean | 2.55 | 19.20 | 45.70 | 7.37 | 11.60 |
| | type | SD | 0.08 | 0.17 | 0.32 | 0.07 | 0.07 |
| | TG | Mean | 0.23 | 24.31 | 46.74 | 7.08 | 8.25 |
| | POS | SD | 0.06 | 0.30 | 0.21 | 0.67 | 0.26 |
| 95Y40/ | 2 | Wild | 2.80 | 20.93 | 45.80 | 6.00 | 10.83 |
| BC75638779 | | type | | | | | |
| 95Y40/ | 8 | Wild | 2.97 | 20.95 | 45.59 | 6.23 | 11.15 |
| BC75638779 | | type | | | | | |
| 95Y40/ | 3 | Wild | 3.26 | 20.53 | 41.63 | 7.77 | 12.43 |
| BC75638779 | | type | | | | | |
| 95Y40/ | 26 | Wild | 3.33 | 20.61 | 41.29 | 7.11 | 12.73 |
| BC75638779 | | type | | | | | |
| 95Y40/ | 9 | Wild | 3.35 | 21.03 | 42.59 | 6.73 | 12.67 |
| BC75638779 | | type | | | | | |
| 95Y40/ | 1 | Wild | 3.35 | 19.70 | 44.88 | 7.39 | 12.05 |
| BC75638779 | | type | | | | | |
| 95Y40/ | 4 | Wild | 3.36 | 19.76 | 44.11 | 6.48 | 12.19 |
| BC75638779 | | type | | | | | |
| 95Y40/ | 19 | Wild | 3.45 | 20.82 | 40.56 | 7.08 | 12.88 |
| BC75638779 | | type | | | | | |
| 95Y40/ | 16 | Wild | 3.62 | 20.73 | 41.74 | 6.97 | 12.22 |
| BC75638779 | | type | | | | | |
| 95Y40/ | 12 | Wild | 3.64 | 20.42 | 41.73 | 7.47 | 13.13 |
| BC75638779 | | type | | | | | |
| 95Y40/ | 18 | Wild | 3.68 | 20.45 | 43.42 | 6.48 | 12.15 |
| BC75638779 | | type | | | | | |
| 95Y40/ | 11 | Wild | 3.89 | 22.17 | 39.33 | 7.14 | 13.14 |
| BC75638779 | | type | | | | | |
| 95Y40/ | 27 | TG | −0.23 | 23.67 | 46.40 | 7.84 | 8.83 |
| BC75638779 | | POS | | | | | |
| 95Y40/ | 5 | TG | 0.00 | 24.35 | 43.52 | 8.29 | 9.40 |
| BC75638779 | | POS | | | | | |
| 95Y40/ | 6 | TG | 0.20 | 24.43 | 46.23 | 6.58 | 7.84 |
| BC75638779 | | POS | | | | | |
| 95Y40/ | 14 | TG | 0.27 | 26.64 | 42.45 | 6.87 | 8.92 |
| BC75638779 | | POS | | | | | |
| 95Y40/ | 7 | TG | 0.32 | 25.39 | 43.93 | 6.64 | 8.78 |
| BC75638779 | | POS | | | | | |
| 95Y40/ | 17 | TG | 0.32 | 24.97 | 45.10 | 7.00 | 8.76 |
| BC75638779 | | POS | | | | | |
| 95Y40/ | 22 | TG | 0.36 | 26.48 | 43.02 | 7.06 | 9.01 |
| BC756387790 | | POS | | | | | |
| 95Y40/ | 13 | TG | 0.47 | 27.24 | 39.30 | 7.48 | 9.87 |
| BC75638779 | | POS | | | | | |
| 95Y40/ | 15 | TG | 0.51 | 25.11 | 44.13 | 7.30 | 8.46 |
| BC75638779 | | POS | | | | | |
| 95Y40/ | 23 | TG | 0.57 | 24.12 | 47.16 | 6.50 | 8.36 |
| BC75638779 | | POS | | | | | |
| 95Y40/ | 10 | TG | 0.59 | 26.18 | 41.61 | 7.17 | 9.74 |
| BC75638779 | | POS | | | | | |
| 95Y40/ | 20 | TG | 0.68 | 24.58 | 45.89 | 6.26 | 8.60 |
| BC75638779 | | POS | | | | | |
| 95Y40/ | 21 | TG | 0.73 | 25.71 | 44.06 | 6.13 | 8.47 |
| BC75638779 | | POS | | | | | |
| 95Y40/ | 24 | TG | 0.84 | 25.25 | 44.37 | 6.73 | 9.01 |
| BC75638779 | | POS | | | | | |
| 95Y40/ | 25 | TG | 0.73 | 26.18 | 39.21 | 7.48 | 10.19 |
| BC75638779 | | POS | | | | | |
| | Wild | Mean | 3.39 | 20.67 | 42.72 | 6.90 | 12.30 |
| | type | SD | 0.30 | 0.64 | 2.05 | 0.53 | 0.72 |
| | TG | Mean | 0.42 | 25.35 | 43.76 | 7.02 | 8.95 |
| | POS | SD | 0.29 | 1.03 | 2.38 | 0.59 | 0.63 |
| 98Y11/ | 16 | Wild | 0.37 | 21.42 | 40.03 | 8.65 | 11.11 |
| BC75638838 | | type | | | | | |
| 98Y11/ | 5 | Wild | 0.55 | 16.05 | 45.20 | 7.69 | 11.80 |
| BC75638838 | | type | | | | | |
| 98Y11/ | 13 | Wild | 1.45 | 19.68 | 40.29 | 8.60 | 12.75 |
| BC75638838 | | type | | | | | |
| 98Y11/ | 12 | Wild | 1.71 | 19.42 | 43.92 | 8.18 | 12.13 |
| BC75638838 | | type | | | | | |
| 98Y11/ | 6 | Wild | 1.83 | 19.81 | 39.92 | 8.73 | 13.17 |
| BC75638838 | | type | | | | | |
| 98Y11/ | 11 | Wild | 2.15 | 17.52 | 38.76 | 8.27 | 13.18 |
| BC75638838 | | type | | | | | |
| 98Y11/ | 10 | Wild | 2.63 | 18.03 | 47.73 | 6.66 | 11.19 |
| BC75638838 | | type | | | | | |
| 98Y11/ | 7 | Wild | 3.13 | 17.11 | 44.58 | 7.43 | 12.64 |
| BC75638838 | | type | | | | | |
| 98Y11/ | 4 | TG | −0.66 | 22.72 | 46.17 | 7.93 | 8.96 |
| BC75638838 | | POS | | | | | |
| 98Y11/ | 14 | TG | −0.59 | 23.01 | 46.62 | 7.74 | 8.64 |
| BC75638838 | | POS | | | | | |
| 98Y11/ | 15 | TG | −0.36 | 21.30 | 49.38 | 7.01 | 8.05 |
| BC75638838 | | POS | | | | | |
| 98Y11/ | 2 | TG | −0.34 | 21.81 | 48.48 | 7.65 | 8.40 |
| BC75638838 | | POS | | | | | |
| 98Y11/ | 1 | TG | −0.13 | 20.70 | 47.63 | 7.55 | 8.83 |
| BC75638838 | | POS | | | | | |
| 98Y11/ | 8 | TG | 0.32 | 20.93 | 48.73 | 7.61 | 9.35 |
| BC75638838 | | POS | | | | | |
| 98Y11/ | 3 | TG | −0.23 | 18.19 | 44.31 | 7.84 | 9.31 |
| BC75638838 | | POS | | | | | |
| | Wild | Mean | 1.72 | 18.63 | 42.55 | 8.03 | 12.25 |
| | type | SD | 0.95 | 1.75 | 3.22 | 0.72 | 0.82 |
| | TG | Mean | −0.28 | 21.24 | 47.33 | 7.62 | 8.79 |
| | POS | SD | 0.3 | 1.6 | 1.8 | 0.3 | 0.5 |

TABLE 6B
SS-NIR compositions (fatty acid profile/weight/moisture) for segregating seed resulting
from backcrosses of an Oil 119 event as described in Table 6A. This construct conferred
a low stachyose/high oil phenotype in three elite female soybean lines. conferring a low
stachyose/high oil phenotype into three elite female soybean lines (as in Table 6A).

| | Plant | SSNIR | | | | | | | |
|---|---|---|---|---|---|---|---|---|---|
| Female/Male | ID | zyg call | Oleic | Linolenic | Palmitic | Stearic | Linoleic | Weight | Moisture |
| 92Y51/ | 4 | Wild | 22.17 | 8.02 | 11.60 | 4.25 | 53.31 | 0.19 | 6.0 |
| BC75638705 | | type | | | | | | | |
| 92Y51/ | 3 | Wild | 21.71 | 8.48 | 11.34 | 4.07 | 51.09 | 0.20 | 5.9 |
| BC75638705 | | type | | | | | | | |
| 92Y51/ | 2 | TG | 33.05 | 2.85 | 10.66 | 5.45 | 44.21 | 0.20 | 6.9 |
| BC75638705 | | POS | | | | | | | |
| 92Y51/ | 5 | TG | 34.75 | 3.48 | 10.60 | 4.92 | 43.52 | 0.21 | 6.9 |
| BC75638705 | | POS | | | | | | | |
| 92Y51/ | 1 | TG | 36.47 | 2.73 | 9.83 | 4.56 | 42.74 | 0.21 | 5.4 |
| BC75638705 | | POS | | | | | | | |
| | Wild | Mean | 21.94 | 8.25 | 11.47 | 4.16 | 52.20 | 0.20 | 6.0 |
| | type | SD | 0.32 | 0.33 | 0.18 | 0.13 | 1.57 | 0.01 | 0.1 |
| | TG | Mean | 34.76 | 3.02 | 10.37 | 4.98 | 43.49 | 0.21 | 6.4 |
| | pos | SD | 1.71 | 0.40 | 0.46 | 0.45 | 0.74 | 0.01 | 0.8 |
| 95Y40/ | 2 | Wild | 32.90 | 6.41 | 11.24 | 4.36 | 42.02 | 0.19 | 6.8 |
| BC75638779 | | type | | | | | | | |
| 95Y40/ | 8 | Wild | 34.64 | 6.11 | 11.09 | 4.43 | 41.30 | 0.18 | 6.6 |
| BC75638779 | | type | | | | | | | |
| 95Y40/ | 3 | Wild | 22.58 | 8.03 | 11.44 | 4.25 | 50.17 | 0.21 | 6.6 |
| BC75638779 | | type | | | | | | | |
| 95Y40/ | 26 | Wild | 23.73 | 7.98 | 11.58 | 4.72 | 49.01 | 0.17 | 6.7 |
| BC75638779 | | type | | | | | | | |
| 95Y40/ | 9 | Wild | 24.03 | 7.19 | 12.02 | 4.42 | 50.14 | 0.21 | 6.6 |
| BC75638779 | | type | | | | | | | |
| 95Y40/ | 1 | Wild | 23.89 | 7.62 | 11.73 | 4.28 | 49.41 | 0.21 | 7.1 |
| BC75638779 | | type | | | | | | | |
| 95Y40/ | 4 | Wild | 26.36 | 7.61 | 11.45 | 3.94 | 47.83 | 0.22 | 6.8 |
| BC75638779 | | type | | | | | | | |
| 95Y40/ | 19 | Wild | 24.70 | 7.41 | 11.70 | 4.20 | 49.63 | 0.19 | 6.6 |
| BC75638779 | | type | | | | | | | |
| 95Y40/ | 16 | Wild | 22.89 | 7.70 | 11.66 | 4.34 | 50.73 | 0.19 | 6.4 |
| BC75638779 | | type | | | | | | | |
| 95Y40/ | 12 | Wild | 22.76 | 7.96 | 11.65 | 4.35 | 50.26 | 0.18 | 6.4 |
| BC75638779 | | type | | | | | | | |
| 95Y40/ | 18 | Wild | 24.39 | 7.36 | 11.54 | 4.00 | 50.68 | 0.20 | 6.7 |
| BC75638779 | | type | | | | | | | |
| 95Y40/ | 11 | Wild | 23.46 | 6.83 | 11.87 | 4.24 | 50.41 | 0.19 | 6.7 |
| BC75638779 | | type | | | | | | | |
| 95Y40/ | 27 | TG | 41.24 | 4.01 | 10.05 | 4.75 | 36.17 | 0.22 | 6.4 |
| BC75638779 | | POS | | | | | | | |
| 95Y40/ | 5 | TG | 36.44 | 4.97 | 10.78 | 4.67 | 39.94 | 0.19 | 6.4 |
| BC75638779 | | POS | | | | | | | |
| 95Y40/ | 6 | TG | 35.61 | 4.92 | 10.92 | 5.37 | 39.35 | 0.19 | 5.8 |
| BC75638779 | | POS | | | | | | | |
| 95Y40/ | 14 | TG | 33.49 | 4.33 | 10.96 | 5.54 | 41.65 | 0.16 | 6.3 |
| BC75638779 | | POS | | | | | | | |
| 95Y40/ | 7 | TG | 32.12 | 3.92 | 11.06 | 5.46 | 43.11 | 0.16 | 5.9 |
| BC75638779 | | POS | | | | | | | |
| 95Y40/ | 17 | TG | 36.50 | 4.33 | 11.11 | 5.41 | 38.70 | 0.18 | 6.0 |
| BC75638779 | | POS | | | | | | | |
| 95Y40/ | 22 | TG | 32.24 | 4.36 | 11.59 | 5.37 | 42.55 | 0.18 | 5.7 |
| BC756387790 | | POS | | | | | | | |
| 95Y40/ | 13 | TG | 30.25 | 4.54 | 11.72 | 5.86 | 43.04 | 0.14 | 5.5 |
| BC75638779 | | POS | | | | | | | |
| 95Y40/ | 15 | TG | 34.64 | 4.11 | 10.62 | 4.51 | 42.88 | 0.19 | 5.4 |
| BC75638779 | | POS | | | | | | | |
| 95Y40/ | 23 | TG | 29.91 | 4.07 | 11.71 | 5.24 | 45.28 | 0.17 | 5.7 |
| BC75638779 | | POS | | | | | | | |
| 95Y40/ | 10 | TG | 32.52 | 5.77 | 10.92 | 5.63 | 41.43 | 0.15 | 6.0 |
| BC75638779 | | POS | | | | | | | |
| 95Y40/ | 20 | TG | 32.67 | 4.84 | 11.11 | 4.96 | 43.33 | 0.17 | 5.5 |
| BC75638779 | | POS | | | | | | | |
| 95Y40/ | 21 | TG | 30.50 | 4.41 | 11.54 | 5.02 | 44.76 | 0.17 | 5.6 |
| BC75638779 | | POS | | | | | | | |
| 95Y40/ | 24 | TG | 31.37 | 3.93 | 11.86 | 5.19 | 44.08 | 0.17 | 5.8 |
| BC75638779 | | POS | | | | | | | |
| 95Y40/ | 25 | TG | 29.02 | 5.59 | 12.42 | 5.90 | 43.35 | 0.14 | 6.2 |
| BC75638779 | | POS | | | | | | | |
| | Wild | Mean | 25.53 | 7.35 | 11.58 | 4.29 | 48.47 | 0.20 | 6.7 |
| | type | SD | 4.00 | 0.62 | 0.25 | 0.20 | 3.28 | 0.02 | 0.2 |
| | TG | Mean | 33.24 | 4.54 | 11.22 | 5.26 | 41.98 | 0.17 | 5.9 |
| | POS | SD | 3.21 | 0.57 | 0.59 | 0.41 | 2.48 | 0.02 | 0.3 |
| 98Y11/ | 16 | Wild | 25.52 | 9.80 | 12.67 | 6.89 | 42.40 | 0.06 | 7.1 |
| BC75638838 | | type | | | | | | | |
| 98Y11/ | 5 | Wild | 18.07 | 12.28 | 11.70 | 5.24 | 50.34 | 0.15 | 7.2 |
| BC75638838 | | type | | | | | | | |
| 98Y11/ | 13 | Wild | 17.59 | 9.86 | 11.52 | 4.78 | 53.22 | 0.18 | 7.2 |
| BC75638838 | | type | | | | | | | |
| 98Y11/ | 12 | Wild | 25.24 | 8.35 | 11.07 | 4.31 | 49.12 | 0.24 | 6.4 |
| BC75638838 | | type | | | | | | | |
| 98Y11/ | 6 | Wild | 17.72 | 10.47 | 11.76 | 5.20 | 52.48 | 0.17 | 7.1 |
| BC75638838 | | type | | | | | | | |
| 98Y11/ | 11 | Wild | 16.44 | 12.58 | 12.16 | 5.28 | 51.37 | 0.08 | 7.5 |
| BC75638838 | | type | | | | | | | |
| 98Y11/ | 10 | Wild | 26.66 | 7.93 | 11.17 | 4.63 | 47.42 | 0.20 | 6.3 |
| BC75638838 | | type | | | | | | | |
| 98Y11/ | 7 | Wild | 16.02 | 11.66 | 12.24 | 4.67 | 54.64 | 0.17 | 6.4 |
| BC75638838 | | type | | | | | | | |
| 98Y11/ | 4 | TG | 37.51 | 5.42 | 10.93 | 5.44 | 37.70 | 0.20 | 6.2 |
| BC75638838 | | POS | | | | | | | |
| 98Y11/ | 14 | TG | 32.13 | 6.46 | 11.23 | 5.66 | 40.99 | 0.19 | 6.1 |
| BC75638838 | | POS | | | | | | | |
| 98Y11/ | 15 | TG | 33.22 | 6.10 | 10.82 | 5.36 | 40.96 | 0.17 | 5.9 |
| BC75638838 | | POS | | | | | | | |
| 98Y11/ | 2 | TG | 36.06 | 5.05 | 10.69 | 5.28 | 40.20 | 0.17 | 5.6 |
| BC75638838 | | POS | | | | | | | |
| 98Y11/ | 1 | TG | 40.73 | 6.50 | 10.04 | 5.46 | 35.31 | 0.22 | 6.4 |
| BC75638838 | | POS | | | | | | | |
| 98Y11/ | 8 | TG | 35.43 | 5.14 | 10.84 | 5.21 | 40.02 | 0.21 | 5.8 |
| BC75638838 | | POS | | | | | | | |
| 98Y11/ | 3 | TG | 47.17 | 8.22 | 10.52 | 4.96 | 25.80 | 0.19 | 6.1 |
| BC75638838 | | POS | | | | | | | |
| | Wild | Mean | 20.41 | 10.37 | 11.79 | 5.12 | 50.13 | 0.16 | 6.9 |
| | type | SD | 4.54 | 1.72 | 0.55 | 0.79 | 3.88 | 0.06 | 0.5 |
| | TG | Mean | 37.46 | 6.13 | 10.72 | 5.34 | 37.29 | 0.19 | 6.00 |
| | POS | SD | 5.1 | 1.1 | 0.4 | 0.2 | 5.5 | 0.0 | 0.3 |

### Example 4

In the following example FT-NIR is used to analyze seed of sample sizes of about 50 seeds to 250 seeds.

Development of NIR Models for FT-NIR

Spectral analyses and capture were performed on a Bruker Multi-Purpose Analyzer (MPA) Fourier Transformed Near Infrared (FT-NIR) spectrometer fitted with a 54 mm diameter rotating cup assembly. Sample sizes of as few as 50 seeds (approximately 10 g of seed) to a full cup load (approximately 53 g of seed) were used, with a sample size of approximately 100 seed (20 g) used where possible. The weight of each sample (to an accuracy of 0.01 g) was recorded prior to scanning. The reflected spectra were captured for each sample to a resolution of 8 cm⁻¹ in the wave length range between 833 and 2778 nm with the instrument in Macro-Reflectance mode. The cup was rotated over the source and detector while sixty-four full spectral scans were collected. The rotation of the cup was stopped and the beans were poured into a foil pan and then returned to the cup prior to scanning for a second time. Three full scan cycles (with complete mixing of the sample between each scan) was found to provide good data quality and sample throughput. Captured spectra were analyzed and models were developed using the Bruker OPUS 7.0 software package. Spectral regions utilized for the prediction of stachyose with the Bruker MPA after model optimization were 1157-1283 nm and 1437-2254 nm.

TABLE 7
Statistics of FT-NIR calibration curves. The number of
reference chemistry measurements are shown in column n.
Range (wt. %) shows the minimum and maximum reference
method measured value in the samples for each constituent.

| Constituent | n | Range | R² | RMSEC | RMSECV |
|---|---|---|---|---|---|
| moisture | 811 | 0-47.2 | 0.99 | 0.40 | 0.44 |
| protein | 102 | 32.1-40.1 | 0.91 | 0.56 | 0.67 |
| oil | 114 | 15.7-23.0 | 0.96 | 0.28 | 0.39 |
| oleic | 1080 | 20.3-89.6 | 0.98 | 2.74 | 3.01 |
| linoleic | 1080 | 1.2-55.6 | 0.98 | 2.51 | 2.67 |
| linolenic | 1080 | 0.9-9.4 | 0.87 | 0.82 | 0.98 |
| stearic | 1080 | 3.1-7.1 | 0.77 | 0.35 | 0.39 |
| palmitic | 1080 | 1.9-12.4 | 0.83 | 0.84 | 0.91 |
| stachyose | 1080 | 0.1-4.9 | 0.78 | 0.66 | 0.73 |
| sucrose | 1080 | 2.15-9.57 | 0.82 | 0.61 | 0.70 |
| total soluble | 1080 | 5.8-12.1 | 0.85 | 0.53 | 0.58 |
| carbohydrates | | | | | |

TABLE 8
FT-NIR measured compositions for ~20 g batches of homozygous positive and null events of Soil
91 (Soil 91-1, Soil 91-2 and Soil 92-1) soybeans. Values presented are means and standard deviations
for two positive (pos) and two null (null) replicates for each event. The delta values indicate
the difference between the transgenic positive and null means for each component.

| | | | | | | | Oleic | Linolenic | | Total soluble |
|---|---|---|---|---|---|---|---|---|---|---|
| Sample | Method | | | Stachyose | Oil | Protein | acid | acid | Sucrose | carbohydrate |
| Soil | FT-NIR | Pos | Mean | 1.9 | 21.3 | 36.8 | 38.2 | 5.8 | 3.4 | 7.2 |
| 91-1 | | | SD | 0.0 | 0.1 | 0.4 | 0.8 | 0.3 | 0.0 | 0.0 |
| | | Null | Mean | 4.5 | 17.5 | 33.7 | 25.1 | 8.1 | 4.9 | 11.0 |
| | | | SD | 0.3 | 0.2 | 1.6 | 0.4 | 0.2 | 0.9 | 0.5 |
| | | | Delta | −2.6 | 3.9 | 3.1 | 13.1 | −2.2 | −1.5 | −3.8 |
| | Ref | Pos | Mean | 0.3 | 21.3 | 38.0 | 24.3 | 5.4 | 4.8 | 6.2 |
| | Chem | | SD | 0.3 | 1.2 | 0.9 | 3.6 | 0.6 | 0.3 | 0.6 |
| | | Null | Mean | 3.5 | 14.9 | 34.5 | 19.1 | 9.3 | 5.4 | 10.2 |
| | | | SD | 0.1 | 1.7 | 1.6 | 0.7 | 0.0 | 1.0 | 1.2 |
| | | | Delta | −3.2 | 6.4 | 3.4 | 5.2 | −3.9 | −0.6 | −4.0 |
| Soil | FT-NIR | Pos | Mean | 1.6 | 20.9 | 37.2 | 39.0 | 5.2 | 3.6 | 7.0 |
| 91-2 | | | SD | 0.4 | 0.9 | 0.6 | 0.1 | 0.6 | 0.4 | 1.1 |
| | | Null | Mean | 4.7 | 17.1 | 32.6 | 26.1 | 7.8 | 4.9 | 11.5 |
| | | | SD | 0.2 | 0.7 | 0.6 | 0.1 | 0.4 | 0.4 | 0.6 |
| | | | Delta | −3.1 | 3.8 | 4.6 | 12.8 | −2.6 | −1.3 | −4.5 |
| | Ref | Pos | Mean | 0.1 | 18.8 | 38.2 | 24.6 | 5.2 | 4.9 | 6.1 |
| | Chem | | SD | 0 | 1.5 | 0.3 | 3.9 | 0.7 | 0.2 | 0.2 |
| | | Null | Mean | 3.4 | 15.7 | 34.2 | 17.9 | 9.5 | 5.2 | 9.9 |
| | | | SD | 0.1 | 2.1 | 0.5 | 0.3 | 0 | 0.1 | 0.4 |
| | | | Delta | −3.3 | 3.1 | 4.1 | 6.7 | −4.3 | −0.3 | −3.8 |
| Soil | FT-NIR | Pos | Mean | 1.84 | 23.60 | 36.39 | 43.64 | 2.47 | 3.40 | 7.64 |
| 92-1 | | | SD | 0.40 | 0.58 | 2.68 | 4.07 | 1.00 | 0.12 | 0.34 |
| | | Null | Mean | 4.54 | 17.39 | 31.89 | 26.78 | 7.82 | 5.23 | 12.09 |
| | | | SD | 0.62 | 0.18 | 1.22 | 2.82 | 0.10 | 0.26 | 0.62 |
| | | | Delta | −2.7 | 6.2 | 4.5 | 16.9 | −5.3 | −1.8 | −4.5 |
| | Ref | Pos | Mean | 2.78 | 20.70 | 39.28 | 30.22 | 3.78 | 1.89 | 5.70 |
| | Chem | | SD | 0.56 | 0.73 | 2.72 | 6.96 | 0.52 | 0.85 | 0.44 |
| | | Null | Mean | 3.53 | 15.98 | 33.16 | 20.07 | 8.56 | 5.82 | 11.00 |
| | | | SD | 0.07 | 0.97 | 1.30 | 1.44 | 0.79 | 0.41 | 0.68 |
| | | | Delta | −0.8 | 4.7 | 6.1 | 10.2 | −4.8 | −3.9 | −5.3 |

The FT-NIR methods used in this example enable detection of transgenic positive material despite discrepancies between the predicted and reference chemistry measured compositions.

Further NIR Models for FT-NIR

FT-NIR measurements for stachyose and other components were taken from 13,881 field grown samples which were screened using NIT to access the compositional diversity of the sample set. Samples that represented the extreme concentrations (both high and low) were selected along with material that was evenly distributed across the intermediate concentrations for each component. Further selections were made to maximize genetic diversity in the samples, along with samples that were clear outliers (i.e., those having measured compositions that were outside the expected ranges). A final set of approximately 400 samples resulted. Spectra were captured on the FT-NIR, as described above. The samples will be analyzed by reference chemistry to determine the concentrations of each constituent and the data will be used to refine the calibrations to facilitate accurate determinations of the sucrosyl-oligosaccharides and other constituents.

### Example 5

Development of NIR Models for Near Infrared Transmittance (NIT).

NIR Spectra, from 850-1050 nm (2-nm step; 30-mm path length), for 400-500 g bulk samples of intact soybeans were acquired in transmission mode using a Foss Tecator AB model 1241 grain analyzer (commercially available from Foss Tecator AB, Höganäs, Sweden) fitted with a standard instrument hopper and sample transport mechanism. The average NIR absorption spectrum for a given sample was arrived at by duplicate analyses each using 10 subsample scans.

All data analysis was performed using InfraSoft International (ISI) chemometrics software WinISI II v.1.50e (commercially available from NIRSystems Inc., Silver Spring, MD, USA), MATLAB 7.10.0 R2010a with Neural Network Toolbox (Mathworks, 2010) and ANN Trainer v1.0a12 (Foss Tecatur AB, 2002) software. Pre-treatment of the raw NIR (log 1/Transmittance) spectral data (850-1050 nm) included multiplicative scatter correction, mean centering and unit vector scaling. Oil and protein content (corrected to a 13% moisture basis) were measured according to techniques developed by USDA-FGIS\GIPSA. Models for palmitic acid, stearic acid, oleic acid, linoleic acid, linolenic acid, sucrose, stachyose and total soluble sugars were developed using Artificial Neural Network (ANN) techniques utilizing the transformed spectrum captured from material presenting a wide compositional diversity for these components. The reference chemistry used for the calibrations was obtained, following spectral capture, using the bulk methods described in Example 2. All calibration development work was performed using standard ANN algorithms available in the software. A Levenberg-Marquardt training function was used with log-sigmoid input and pure-linear output transfer functions. Between four and nine neurons were used in one hidden node layer.

The optimum number of iterations (epochs) was chosen when the randomly selected test set error was minimized. The coefficient of determination (R²) was used to describe the correlation between reference (observed) and NIR-predicted values for the calibration set. The Ratio of Performance to Deviation (RPD), defined as the ratio of the SD of the reference values to the SECV (or test set Standard Error of Prediction (SEP)), was used as a normalized indicator for comparing NIR models.

NIT wavelengths useful in the prediction of stachyose concentration in whole soybeans were 850, 866, 880, 890, 902, 910, 920, 930, 944, 952, 964, 978, 990, 1004, 1016, 1032, and 1042 nm. Measurements were taken using the spectra and reference chemistry collected from three years of field grown soybeans from multiple sites within the United States, Argentina, and Puerto Rico. The statistics for the accuracy of the non-destructive NIR methods compared with the standard methods disclosed herein are shown in Table 9.

TABLE 9
Statistics of accuracy of NIT measurements. n = number of reference
chemistry measurements used for each constituent comparison between
the NIR methods compared with the standard methods disclosed herein.

| Constituent | n | Range | R² | RMSEC | RMSECV |
|---|---|---|---|---|---|
| moisture (approx.) | 379 | | 0.99 | 0.30 | 0.30 |
| protein | 266 | 31.9-51.0 | 0.97 | 0.54 | 0.54 |
| oil | 103 | 16.9-25.0 | 0.96 | 0.35 | 0.35 |
| oleic | 2277 | 19.2-91.9 | 0.995 | 1.59 | 1.56 |
| linoleic | 2190 | 0.2-58.4 | 0.98 | 2.19 | 2.20 |
| linolenic | 2277 | 0.9-10.3 | 0.96 | 0.46 | 0.31 |
| stearic | 2276 | 2.6-9.5 | 0.86 | 0.41 | 0.33 |
| palmitic | 2277 | 1.79-12.5 | 0.96 | 0.52 | 0.51 |
| stachyose | 354 | 0.05-4.9 | 0.89 | 0.49 | 0.57 |
| sucrose | 354 | 2.2-9.9 | 0.90 | 0.48 | 0.39 |
| total soluble | 354 | 5.7-12.1 | 0.88 | 0.48 | 0.42 |
| carbohydrates | | | | | |

Values for stachyose measured using NIT and reference chemistry for bulk samples of transgenic positive and negative events of Soil 19 are given in Table 10.

TABLE 10
NIT predicted stachyose content of bulk samples of transgenic positive
and negative events of Soil 19. The beans were harvested from plants
grown at 9 independent mid-Western sites. The samples were subjected
to reference chemistry after the NIT spectra had been captured.

| | | | Ref Chem | NIT |
|---|---|---|---|---|
| | Trait | | Stachyose | Stachyose |
| Event | Call | 2013 EUid | (wt. %) | (wt. %) |
| Soil19 1.2.1 | Pos | 242479091 | 0.45 | 0.25 |
| Soil19 1.2.1 | Neg | 242479093 | 3.62 | 3.40 |
| | | Δ between mean NIT | | 0.20 |
| | | measurement and the | | |
| | | reference chem value | | |
| Soil19 2.2.1 | Neg | 219154850 | 3.59 | 3.61 |
| Soil19 2.2.1 | Neg | 219154734 | 3.76 | 3.54 |
| Soil19 2.2.1 | Neg | 219154901 | 4.53 | 4.26 |
| Soil19 2.2.1 | Neg | 219154925 | 4.23 | 4.37 |
| Soil19 2.2.1 | Neg | 219154941 | 4.01 | 3.43 |
| Soil19 2.2.1 | Neg | 219154949 | 4.47 | 3.93 |
| Soil19 2.2.1 | Neg | 219154957 | 3.49 | 3.74 |
| Soil19 2.2.1 | Neg | 219154981 | 4.34 | 3.57 |
| | | Mean | 4.05 | 3.81 |
| | | SD | 0.40 | 0.35 |
| | | Δ between mean NIT | | 0.25 |
| | | measurement and the | | |
| | | reference chem value | | |
| Soil19 2.2.1 | Pos | 219154736 | 1.03 | 1.38 |
| Soil19 2.2.1 | Pos | 219154769 | 0.51 | 0.20 |
| Soil19 2.2.1 | Pos | 219154781 | 1.00 | 1.02 |
| Soil19 2.2.1 | Pos | 219154832 | 0.88 | 1.01 |
| Soil19 2.2.1 | Pos | 219154842 | 1.01 | 0.94 |
| Soil19 2.2.1 | Pos | 219154886 | 1.03 | 0.85 |
| Soil19 2.2.1 | Pos | 219154918 | 0.46 | 0.27 |
| Soil19 2.2.1 | Pos | 244113717 | 1.01 | 1.02 |
| | | Mean | 0.87 | 0.84 |
| | | SD | 0.24 | 0.40 |
| | | Δ between mean NIT | | 0.03 |
| | | measurement and the | | |
| | | reference chem value | | |
| Soil19 5.3.3 | Neg | 219154682 | 3.73 | 3.43 |
| Soil19 5.3.3 | Neg | 219154848 | 3.52 | 3.62 |
| Soil19 5.3.3 | Neg | 219154923 | 4.10 | 4.06 |
| Soil19 5.3.3 | Neg | 219154939 | 4.23 | 3.41 |
| Soil19 5.3.3 | Neg | 219154947 | 4.66 | 3.41 |
| Soil19 5.3.3 | Neg | 219154955 | 3.53 | 3.89 |
| Soil19 5.3.3 | Neg | 219154979 | 4.33 | 3.33 |
| Soil19 5.3.3 | Neg | 244113714 | 3.68 | 3.76 |
| | | Mean | 3.97 | 3.61 |
| | | SD | 0.42 | 0.27 |
| | | Δ between mean NIT | | 0.36 |
| | | measurement and the | | |
| | | reference chem value | | |
| Soil19 5.3.3 | Pos | 242479108 | 0.84 | 0.92 |
| Soil19 5.3.3 | Pos | 242479110 | 0.57 | 0.50 |
| Soil19 5.3.3 | Pos | 219154766 | 0.30 | 0.21 |
| Soil19 5.3.3 | Pos | 219154778 | 0.74 | 0.59 |
| Soil19 5.3.3 | Pos | 219154830 | 0.51 | 0.22 |
| Soil19 5.3.3 | Pos | 219154838 | 0.72 | 0.93 |
| Soil19 5.3.3 | Pos | 219154884 | 0.74 | 0.64 |
| Soil19 5.3.3 | Pos | 219154900 | 0.80 | 1.59 |
| Soil19 5.3.3 | Pos | 219154916 | 0.41 | 0.25 |
| Soil19 5.3.3 | Pos | 219154940 | 0.44 | 0.55 |
| Soil19 5.3.3 | Pos | 244113715 | 0.83 | 0.35 |
| | | Mean | 0.61 | 0.59 |
| | | SD | 0.19 | 0.44 |
| | | Δ between mean NIT | | 0.02 |
| | | measurement and the | | |
| | | reference chem value | | |

The data show that the NIT measurements of stachyose in the transgenic positive events was <1.6% whereas the minimal measured value for the transgenic negative (null) events was 3.3%, allowing distinctions between the transgenic positive and negative events to be made, based on the stachyose measurements alone. The average stachyose NIT measurements for both the transgenic positive and negative events were within 0.4% of those measured by reference chemistry (Table 10) showing a high degree of precision (ability to differentiate between transgenic positive and negative events) and accuracy (closeness to the reference chemistry determined value).

### Example 6

The use of Near Infrared Transmittance (NIT) Spectroscopy to Identify Material for Improved Accuracy of Measurements made with NIR or NIT

The development of robust spectroscopic models for the identification of transgenic materials that have been altered in their composition is dependent on several factors:

The sample sizes used for NIT analysis in this example were large enough to be subjected to industry standard reference chemistry methods (which typically require more than 60 g of seed). Measurements taken using NIT were scalable and were transferred between NIR and NIT instruments. The sample size used for NIT (400-500 g) was compatible with growing transgenic and breeding lines in short (2 to 3 meter) field plots. Field culture allowed for the introduction of environmental variation to be accounted for i.e., representatives of the same events (such as transgenes or genetic modifications) grown in different states and under different field conditions. The sample size was sufficient to provide material to calibrate instruments with smaller sample size requirements i.e., the FT-NIR and SS-NIR, single seed reference chemistry used for SS-NIR.

NIT spectroscopy was used to analyze 3692 samples containing 400-500 g of seed grown in field plots Johnston, Iowa during the 2014 season. Compositional data for each of the 11 constituents listed in Table 11 were collected. The data was analyzed by plotting the entire ranges of composition for each component. Samples that represented the extreme concentrations (both high and low) were selected along with material that was evenly distributed across the intermediate concentrations for each component. Further selections were made to maximize genetic diversity in the samples, along with samples that were clear outliers (i.e., those having measured compositions that were outside the expected ranges). This process resulted in the selection of 183 samples for further analysis; i.e., approximately 5% of the initial set. The subset was then analyzed by SS-NIR, FT-NIR (on both a Bruker MPA and Tango FT=NIR spectrometers) prior to being ground and subjected to reference chemistry.

Another set of 2020 soybean samples containing 400-500 g of seed grown in Argentina field plots during the 2015-2016 growing season were scanned on a Foss 1241 NIT spectrometer. Compositional data for the 11 constituents in Table 11 was generated using the collected absorption spectra. A selection of 139 calibration expansion samples was made based on uniform predicted composition, and individual sample spectra compared to the model spectral database. This procedure utilized principal component analysis of the model database, and the relative similarity of the Argentina set to identify samples not currently represented in the model population. Another set of 40 samples intended to validate the model performance was selected based on wide ranging, uniform estimated concentration and the presence of similar samples in the model or currently being selected for calibration expansion.

Each sample was scanned on a Bruker MPA and then on a Bruker Tango (Fourier Transform Near Infra-Red (FT-NIR) spectrometers) each fitted with 54 mm diameter rotating cup assemblies. Twenty grams (approximately 100 seed) samples were removed from the bulk packages, after thorough mixing, and were used for analysis on one of the FT-NIR instruments. The seed samples were then returned to the bulk bags and a second sample was used for spectral capture on the second instrument. The weight of each sample (to an accuracy of 0.01 g) was recorded prior to scanning. The conditions for spectral capture on the Tango instrument were similar to those described for the Bruker MPA (Example 4) except that the wave length range was slightly narrower (867 and 2530 nm on the Tango vs 833 and 2778 nm on the MPA). Captured spectra from both instruments were analyzed and prediction models were developed using the Bruker OPUS 7.0 software package.

Twelve seeds from each sample were selected randomly from the bulk packages and were analyzed by SS-NIR. Individual seed identities were maintained during the spectral capture process. Each bean was then placed into 2 ml polypropylene tubes (Corning Inc, Corning NY, USA; part #430917) previously labeled with unique bar code identifiers, in preparation for single seed reference chemistry analysis (Example 2) and subsequent SS-NIR model refinement.

Seventy-five gram samples were then removed from each of the bulk samples and after grinding the samples were subjected to bulk reference chemistry analysis (see Examples 2). The mean, SD, minimum and maximum values for each component from each analytical platform are given in Table 11.

TABLE 11
Mean, SD, minimum and maximum values soybean compositional components.

| | | | | | | | | | | | | Total soluble |
|---|---|---|---|---|---|---|---|---|---|---|---|---|
| Data | | OIL | PROT | Palmitic | Stearic | Oleic | Linoleic | Linolenic | Sucrose | Raffinose | Stachyose | carbohydrates |
| Source | | wt. % | wt. % | acid | acid | acid | acid | acid | wt. % | wt. % | wt. % | wt. % |
| NIT 3692 | Mean | 19.5 | 36.1 | 11.0 | 4.0 | 24.0 | NA | 5.5 | 5.2 | 0.6 | 2.4 | 8.8 |
| | SD | 1.7 | 0.9 | 1.0 | 0.7 | 9.5 | NA | 2.6 | 0.6 | 0.1 | 1.0 | 0.9 |
| | Min | 16.0 | 33.1 | 4.1 | 0.8 | 12.1 | NA | 1.0 | 2.7 | 0.1 | 0.2 | 5.6 |
| | Max | 24.2 | 40.6 | 14.1 | 9.5 | 88.3 | NA | 16.5 | 7.6 | 1.2 | 4.2 | 11.3 |
| Select | Mean | 19.5 | 36.3 | 10.8 | 4.1 | 28.9 | NA | 5.5 | 5.3 | 0.6 | 2.3 | 8.8 |
| 183 NIT | SD | 1.8 | 1.1 | 1.6 | 0.9 | 18.2 | NA | 2.6 | 0.7 | 0.1 | 1.1 | 1.0 |
| | Min | 16.2 | 33.8 | 4.1 | 2.5 | 13.0 | NA | 1.1 | 2.7 | 0.1 | 0.2 | 5.7 |
| | Max | 24.2 | 40.6 | 14.1 | 9.5 | 88.3 | NA | 16.5 | 7.4 | 1.2 | 4.1 | 11.0 |
| Reference | Mean | NA | 36.8 | 10.6 | 4.3 | 31.2 | 47.0 | 6.0 | 5.8 | 0.6 | 2.6 | 9.5 |
| Chemistry | SD | NA | 1.2 | 2.7 | 0.8 | 17.4 | 14.6 | 3.0 | 1.1 | 0.2 | 1.6 | 1.3 |
| | Min | NA | 33.9 | 2.5 | 2.7 | 16.2 | 1.1 | 1.2 | 2.6 | 0.1 | 0.1 | 6.3 |
| | Max | NA | 39.7 | 19.2 | 6.8 | 86.9 | 57.4 | 9.4 | 9.7 | 1.2 | 5.2 | 13.3 |
| SS-NIR | Mean | 21.5 | 42.2 | 10.0 | 3.9 | 31.0 | 49.0 | 5.5 | 6.3 | NA | 2.5 | 10.1 |
| Means Only | SD | 2.1 | 1.7 | 1.4 | 0.9 | 16.7 | 13.9 | 2.4 | 1.0 | NA | 1.2 | 1.4 |
| | Min | 17.5 | 37.8 | 4.3 | 2.6 | 14.9 | 2.4 | 0.6 | 4.0 | NA | −0.5 | 6.8 |
| | Max | 26.1 | 46.9 | 11.9 | 6.8 | 88.4 | 62.2 | 9.8 | 9.8 | NA | 4.3 | 13.4 |
| FT-NIR | Mean | 19.5 | 36.3 | 8.9 | 4.5 | 31.8 | 44.5 | 5.6 | 6.0 | NA | 2.2 | 9.5 |
| MPA | SD | 1.9 | 1.3 | 1.7 | 0.9 | 16.2 | 13.5 | 2.2 | 0.8 | NA | 1.4 | 1.3 |
| | Min | 16.4 | 33.1 | 3.3 | 3.3 | 17.6 | −0.7 | −1.4 | 3.5 | NA | −1.1 | 5.7 |
| | Max | 23.0 | 40.1 | 11.6 | 6.5 | 86.2 | 55.8 | 8.7 | 9.2 | NA | 4.7 | 11.5 |
| FT-NIR | Mean | 19.1 | 36.6 | 9.0 | 5.2 | 30.6 | 49.2 | 4.4 | 6.1 | NA | 0.8 | 8.3 |
| Tango | SD | 1.6 | 1.4 | 1.6 | 0.9 | 16.7 | 14.2 | 2.1 | 0.7 | NA | 1.4 | 1.2 |
| | Min | 16.6 | 33.0 | 3.2 | 3.6 | 17.1 | 0.1 | −2.0 | 4.1 | NA | −2.4 | 5.1 |
| | Max | 22.4 | 40.2 | 10.5 | 7.6 | 87.2 | 60.4 | 7.3 | 9.0 | NA | 3.0 | 10.5 |

Comparison of the means, minimum and maximum values for the whole sample set (represented by the “Original NIT Values” which were developed for 3692 samples) and those for the selected set of 183 samples shows that the latter set covered the available dynamic range for each of the predicted components (Table 11). Further, the close agreement between the mean, minimum and maximum NIT predicted values for the selected set of 183 samples and the actual compositional contents measured for these samples by reference chemistry indicates the high degree of precision and accuracy of the measurements for most of the components. For example, the predicted mean, minimum and maximum stachyose contents for the 183 sample subset differed from the reference chemistry measured values by −0.3wt %, 0.1wt % and −1.1wt %, respectively. In contrast the predicted mean, minimum and maximum palmitic acid contents for the 183 sample subset differed from the reference chemistry measured values by 0.2 relative %, 1.6 relative % and −5.0 relative %, respectively. Inclusion of the reference chemistry values into the NIT models will lead to improvements in the precision of the measurements (i.e., the ability to differentiate between unknown samples differing in their composition) and the accuracy (the ability to predict compositions that are indistinguishable from those measured using standard reference chemistry methods).

### Example 7

As an illustration of the precision of the reference chemistry assay for sucrose, raffinose and stachyose, bulk samples of beans from three different commodity soybean samples were subjected to analysis as blind duplicates. Six replicates for each duplicated sample were analyzed using the standard analytical methods for total soluble carbohydrate derivatization and analysis according to the methods set forth in Example 2.

Each sugar was quantified relative to its own calibration curve, after dividing each individual peak by the area of the internal standard in each sample and standard. Final carbohydrate concentrations were expressed corrected for moisture content as set forth herein. Residual sucrose, raffinose and stachyose recovered in the starch digestions were included in the total values reported for each sugar.

The average coefficient of variation (mean/standard deviation of the mean, expressed as a percentage) for sucrose, raffinose and stachyose were, 1.43%, 1.34% and 2.11%, respectively. As an illustration of the accuracy of the reference chemistry assay for sucrose, raffinose and stachyose, bulk samples of beans from three different commodity soybean samples were subjected to analysis as blind duplicates using the methods described here and at two contract research organizations. The mean values for sucrose of 4.31+/−0.22 (internal), 4.26+/−0.11, 4.26+/−0.21 were not significantly different at the 95% confidence interval. The mean values for stachyose were 3.45+/−0.13 (internal), 3.16+/−0.14 and 3.36+/−0.27 were not significantly different at the 95% confidence interval. Significant differences were observed in the raffinose values of 0.93+/−0.02 (internal), 0.73+/−0.10 and 0.47+/−0.03.

### Example 8

Analysis of Soybean Meal

Rapid compositional analyses of soybean meals, including analyses reporting on the concentration of anti-nutritional factors such as the sucrosyl oligosaccharide, raffinose and stachyose and nutritionally desirable components such as protein, amino acids and sucrose can be carried out.

Defatted powders remaining after the bulk oil extraction process (Example 2.15) from soybeans are used to capture NIR reflection absorption spectra, either on a Bruker MPA or on a Foss 6500 full spectrum instrument. The integrating sphere channel of the MPA operating in macro reflectance mode is utilized to scan the powders contained within a 15×45 mm borosilicate vial (Qorpak p/n GLC-00982) in triplicate at a resolution of 8 cm-1 from 833-2778nm. Alternately a Foss/NIRSystems 6500 near infrared reflectance instrument equipped with an autosampler attachment will be utilized to scan the powders contained within a 51 mm ring cup in duplicate at a resolution of 2 nm from 400-2500 nm. The samples are analyzed for the concentration of protein, moisture, sucrose, raffinose, stachyose and total soluble carbohydrates using reference chemistry methods described in Example 2. The resulting spectral and chemical data will enable accurate determination of each constituent's concentration.

### Example 9

A diverse set of soybeans grown in the field in North America and Argentina in 2015 and North America in 2016 were selected for their compositional diversity using the methodology described in Example 6. After spectral capture on both the FT-NIR (Example 4) and the NIT (Example 5) platforms the samples were subjected to reference chemistry using the bulk sample methodologies described in Example 2. The statistical characteristics of the models obtained are described in Table 12. In generating the models raffinose and total saturated fatty acids were added as analytes. The moisture models were not updated.

TABLE 12
Statistics of accuracy of NIT measurements. n = number of
reference chemistry measurements used for each constituent
comparison between the NIR methods compared with the
standard methods disclosed herein. Oil, protein and
carbohydrate ranges are presented on a 13% moisture basis.
Fatty acids are presented on a relative % basis.

| Constituent | n | Range | R² | RMSEC | RMSECV |
|---|---|---|---|---|---|
| moisture | 379 | | 0.99 | 0.3 | 0.3 |
| protein | 1101 | 28.3-41.0 | 0.94 | 0.40 | 0.40 |
| oil | 1105 | 15.3-28.4 | 0.98 | 0.30 | 0.31 |
| oleic | 1141 | 14.6-87.3 | 1.00 | 1.40 | 1.51 |
| linoleic | 1141 | 0.8-59.2 | 1.00 | 1.24 | 1.28 |
| linolenic | 1141 | 0.8-10.2 | 0.96 | 0.49 | 0.51 |
| stearic | 1141 | 2.7-16.1 | 0.89 | 0.24 | 0.34 |
| palmitic | 1141 | 2.0-23.1 | 0.96 | 0.63 | 0.52 |
| total sats | 1141 | 4.7-28.0 | 0.98 | 0.51 | 0.52 |
| raffinose | 1200 | 0.1-1.5 | 0.56 | 0.19 | 0.26 |
| stachyose | 1200 | 0.02-5.2 | 0.95 | 0.37 | 0.44 |
| sucrose | 1200 | 1.7-9.9 | 0.72 | 0.62 | 0.67 |
| total soluble | 1200 | 3.7-13.3 | 0.86 | 0.62 | 0.78 |
| carbohydrates | | | | | |

The performance of the stachyose model improved with the addition of the new data; R² 0.95 vs 0.89 (compare Tables 9 and 12); RMSEC 0.37 vs 0.49 (the lower value indicates an improved resolution between samples in the model); RMSECV 0.44 vs 0.57 (a lower value indicates an improved resolution between samples not in the model, i.e., unknowns). The model statistics for sucrose and total soluble carbohydrates did not improve.

### Example 10

Analysis of soybean meals; Defatted Powder Model Derivation

Rapid compositional analyses of soybean meals, including analyses of the concentration of anti-nutritional factors such as the sucrosyl oligosaccharide, raffinose and stachyose and nutritionally desirable components such as sucrose was carried out. Defatted powder was used; defatted soybean flakes could also be used.

Defatted powders remaining after the bulk oil extraction process (Example 2.15) from soybeans were used to capture NIR reflection absorption spectra on a Bruker MPA. The integrating sphere channel of the MPA operating in macro reflectance mode was utilized to scan the powders contained within a 15×45mm borosilicate vial (Qorpak p/n GLC-00982) in triplicate, at a resolution of 8 cm-1 from 800-2778 nm. It is expected that a Foss/NIRSystems 6500 near infrared reflectance instrument equipped with an autosampler attachment could also be utilized to scan the powders contained, for example, within a 51 mm ring cup in duplicate at a resolution of 2 nm from 400-2500 nm.

After spectral capture samples were analyzed for the concentration of protein, moisture, sucrose, raffinose, stachyose and total soluble carbohydrates using reference chemistry methods described in Example 2. The resulting spectral and chemical data were used to generate calibrations for the prediction of the meal compositions.

Captured spectra and accompanying reference chemistry were used to derive Partial Least Squares predictive models utilizing Bruker's Opus 7.0 software package. Individual triplicate spectra were averaged into a single observation prior to the model regression. Spectral regions and absorption data pre-treatment were selected for each analyte using the OPUS optimization algorithm. Raffinose, stachyose, and total soluble carbohydrates employed first derivative and standard normal variate pre-treatment while using regions 1333-2355 nm, 1464-2355 nm, and 1063-1125 nm plus 1465-2355 nm respectively. Sucrose modeling entailed using first derivative and multiplicative scatter correction to pre-treat spectra and incorporated 1639-2355 nm wavelength regions. The resulting model statistics are displayed in Table 13.

TABLE 13
Statistics of FT-NIR calibration curves for Defatted Powders.
The number of reference chemistry measurements are shown
in column n. Range (wt. % corrected to 13% Moisture) shows
the minimum and maximum reference method measured
value in the samples for each constituent.

| Constituent | n | Range | R² | RMSEC | RMSECV |
|---|---|---|---|---|---|
| Raffinose | 231 | 0.15-1.76 | 0.73 | 0.20 | 0.22 |
| Stachyose | 238 | 0.05-5.81 | 0.97 | 0.34 | 0.39 |
| Sucrose | 230 | 3.75-8.34 | 0.85 | 0.34 | 0.38 |
| Total Carbs | 232 | 6.26-13.75 | 0.96 | 0.33 | 0.40 |

The data indicate that stachyose in soybean meals were measured to a similar degree of precision and accuracy as those achieved for whole beans using the methods described here, i.e., R² 0.97 and 0.95, RMSEC 0.34 and 0.37 for the meal and whole bean NIT models (Table 12), respectively.

All publications, patents, and patent applications mentioned in the specification are incorporated by reference herein for the purpose cited to the same extent as if each was specifically and individually indicated to be incorporated by reference herein.

The foregoing invention has been described in detail by way of illustration and example for purposes of clarity and understanding. As is readily apparent to one skilled in the art, the foregoing description represents only some of the methods and compositions that illustrate the embodiments of the foregoing invention. It will be apparent to those of ordinary skill in the art that variations, changes, modifications, and alterations may be applied to the compositions and/or methods described herein without departing from the true spirit, concept, and scope of the invention.

As used herein, the terms “comprises,” “comprising,” “includes,” “including,” “has,” “having,” “contains”, “containing,” “characterized by” or any other variation thereof, are intended to cover a non-exclusive inclusion.

Unless expressly stated to the contrary, “or” is used as an inclusive term. For example, a condition A or B is satisfied by any one of the following: A is true (or present) and B is false (or not present), A is false (or not present) and B is true (or present), and both A and B are true (or present). The indefinite articles “a” and “an” preceding an element or component are nonrestrictive regarding the number of instances (i.e., occurrences) of the element or component. Therefore “a” or “an” should be read to include one or at least one, and the singular word form of the element or component also includes the plural unless the number is obviously meant to be singular.

arated and further used in soybean processing or breeding.

What is claimed is:

1. A method for measuring the amount of stachyose in soybean powder, soybean meal or soybean flakes, the method comprising:
(a) directing near infrared light from a light source onto a soybean powder, soybean meal, or soybean flake sample to form modified light from the soybean powder, soybean meal, or soybean flake sample;
(b) receiving the modified light in an imaging device;
(c) measuring the amount of stachyose in the soybean powder, soybean meal, or soybean flake sample based on the received modified light, the amount of the stachyose being measured to an accuracy that is within 1 wt. % of the amount measured using a standard reference analytical method.

2. The method of claim 1, further comprising transporting the soybean meal, soybean powder or soybean flakes to a first location when the amount of stachyose measured is below a threshold value and transporting the soybean powder, soybean meal or soybean flakes to a different second location when the amount of stachyose measured is at or above the threshold value.

3. The method of claim 2, wherein threshold value is between 2 wt. % and 0.1 wt. %.

4. The method of claim 2, wherein threshold value is between 1.5 wt. % and 0.1 wt %.

5. The method of claim 2, wherein threshold value is between 1 wt. % and 0.1 wt %.

6. The method of claim 2, wherein the soybean meal, soybean powder or soybean flakes above the threshold value differ by a least 1 wt. % stachyose from the soybean meal, soybean powder or soybean flakes below the threshold value.

7. The method of claim 2, wherein the threshold value is about 1 percentage point.

8. The method of claim 2, wherein the modified light comprises reflected light.

9. The method of claim 2, wherein the amount of stachyose is measured in soybean powder.

10. The method of claim 2, wherein the amount of stachyose is measured in soybean meal.

11. The method of claim 2, wherein the amount of stachyose is measured in soybean flakes.

12. The method of claim 2, wherein the amount of stachyose measured in step (c) is an average amount for the soybean powder, soybean meal or soybean flakes.

13. The method of claim 1, wherein the modified light comprises transmitted light.

14. The method of claim 1, wherein the modified light comprises reflected light.

15. The method of claim 1, wherein the amount of stachyose is measured in soybean powder.

16. The method of claim 1, wherein the amount of stachyose is measured in soybean meal.

17. The method of claim 1, wherein the amount of stachyose is measured in soybean flakes.

18. The method of claim 1, wherein the soybean powder, soybean meal or soybean flakes are processed to produce a soy protein concentrate when the amount of sucrosyl-oligosaccharide measured is below a threshold value between 2 wt. % and 0.1 wt. % and wherein the soybean powder, soybean meal or soybean flakes are processed without producing a soy protein concentrate when the amount of sucrosyl-oligosaccharide measured is at or above the threshold value.

19. The method of claim 18, wherein the threshold value between 1.5 wt. % and 0.1 wt. %.

20. The method of claim 18, wherein the threshold value between 1 wt. % and 0.1 wt. %.
